(19) United States
(12) Patent Application Publication
(10) Pub. No.: US 2006/0199864 A1
(43) Pub. Date: Sep. 7, 2006

(54) Selection of pH-dependent compounds for in vivo therapy

(76) Inventors: Stephen F. Traynelis, Decatur, GA (US); Dennis C. Liotta, Atlanta, GA (US); Raymond J. Dingledine, Atlanta, GA (US)

Correspondence Address: KING & SPALDING LLP, 1180 PEACHTREE STREET, ATLANTA, GA, 30309, US

(21) Appl. No.: 11/210,330

(22) Filed: Aug. 23, 2005

Related U.S. Application Data
(60) Provisional application No. 60/603,790, filed on Aug. 23, 2004.
(60) Provisional application No. 60/604,327, filed on Aug. 25, 2004.
(60) Provisional application No. 60/604,972, filed on Aug. 27, 2004.

(51) Int. Cl. A61K 31/18; C07C 311/09

(52) U.S. Cl. 514/602; 564/99

(57) ABSTRACT

This invention is in the area of improved methods for the selection of pH dependent compounds to be used before, during or after a pH-lowering event as a means to minimize or prevent tissue damage.

This invention claims priority to U.S. provisional patent application No. 60/603,790, filed Aug. 23, 2004; 60/604,327, filed Aug. 25, 2004 and 60/604,972, filed Aug. 27, 2004.

### FIELD OF THE INVENTION

This invention is in the area of improved methods for the selection of pH dependent compounds to be used before, during or after a pH-lowering event as a means to minimize or prevent tissue damage.

### BACKGROUND

Nerve cells, or neurons, transmit signals from the environment to the central nervous system (CNS), among different regions of the CNS, and from the CNS back to other organs (i.e., the periphery). This signal transmission is mediated primarily by small molecules called neurotransmitters. In general, neurotransmitters can be classified as either excitatory or inhibitory. Excitatory neurotransmitters increase and inhibitory neurotransmitters decrease the activity (e.g., the firing rate) of the signal-receiving (i.e., postsynaptic) neuron. Neurons differ in their abilities to recognize, integrate, and pass on the signals conveyed by neurotransmitters. For example, some neurons continually fire at a certain rate and thus can either be excited or inhibited in response to environmental changes. Other neurons normally are at rest in the absence of external stimulation. Accordingly, any modification of their activity must occur in the form of excitation. As a result, neuronal excitation plays a fundamental role in controlling brain functioning. Of the numerous molecules governing normal brain functioning, glutamate (also called glutamic acid) is one of the most important. Research on its functions has generated significant advances in understanding how the brain works. Glutamate's role as an important signaling molecule has been recognized only within the past two decades.

Glutamate is an amino acid. Glutamate, as other amino acids, is present throughout the brain in relatively high concentrations. Consequently, researchers initially thought that glutamate was primarily an intermediate metabolic product of many cellular reactions unrelated to neuronal signal transmission and thus did not interpret its presence in neurons as evidence of a potential role as a neurotransmitter. The first indications of glutamate's excitatory function in the brain emerged in the 1950's, however, these findings were initially dismissed because glutamate application to neurons elicited excitatory responses in virtually every brain area examined, suggesting that this excitation was not a specific response. Only later did scientists recognize that the observed effects of glutamate were indeed valid because they could be attributed to the activation of excitatory receptors present throughout the CNS. In the 1970's and 1980's, researchers identified specific glutamate receptors, i.e. proteins on the surface of neurons that specifically bind glutamate secreted by other neurons and thereby initiate the events that lead to the excitation of the postsynaptic neuron. The identification of these glutamate receptors underscored glutamate's importance as an excitatory neurotransmitter.

Knowledge of the glutamatergic synapse has advanced tremendously in the last 10 years, primarily through application of molecular biological techniques to the study of glutamate receptors and transporters. It is now known that there are three families of ionotropic receptors with intrinsic cation permeable channels, N-methyl-D-aspartate (NMDA), alpha-amino-3-hydroxy-5-methyl-4-isoxazolepropionic acid (AMPA) and kainate receptors. There are also three groups of metabotropic, G protein-coupled glutamate receptors (mGluR) that modify neuronal and glial excitability through G protein subunits acting on membrane ion channels and second messengers such as inositol tris phosphate and cAMP. In addition, there are also two glial glutamate transporters and three neuronal transporters in the brain.

Glutamate is essential for normal brain function. Glutamate plays a primary role in the control of cognition, motor function, synaptic plasticity, learning and memory. High levels of endogenous glutamate, through its overactivation of NMDA, AMPA or mGluR1 receptors, can contribute to brain damage. Examples of brain damage associated with excess glutamate or excitotoxicity are seen after status epilepticus, cerebral ischemia and traumatic brain injury. Excitotoxicity (e.g., toxicity caused by the overactivation of glutamate receptors) also contributes to chronic neurodegeneration in such disorders as Parkinson's disease, Alzheimer's disease, amyotrophic lateral sclerosis, retinal degeneration and Huntington's chorea. In animal models of cerebral ischemia and traumatic brain injury, NMDA and AMPA receptor antagonists protect against acute brain damage and delayed behavioral deficits. Other clinical conditions that may respond to drugs acting on glutamatergic transmission include epilepsy, amnesia, anxiety, hyperalgesia and psychosis (Meldrum B S. J Nutr. 2000; 130(4S Suppl): 1007S-15 S).

NMDA Receptor Antagonists

The NMDA subtype of glutamate-gated ion channels mediates excitatory synaptic transmission between neurons in the central nervous system (Dingledine et al. (1999), Pharmacological Reviews 51:7-61). NMDA receptors are composed of NR1, NR2 (A, B, C, and D), and NR3 (A and B) subunits, which determine the functional properties of native NMDA receptors. Expression of the NR1 subunit alone does not produce a functional receptor. Co-expression of one or more NR2 subunits is required to form functional channels. In addition to glutamate, the NMDA receptor requires the binding of a co-agonist, glycine, to allow the receptor to function. The glycine binding site is found on the NR1 subunit, whereas the glutamate binding site is found on NR2 subunits. The NR3 subunit also binds glycine. The NR2B subunit also possesses a binding site for spermine-like polyamines, which are regulatory molecules that modulate the functioning of the NMDA receptor. At resting membrane potentials, NMDA receptors are largely inactive. This is due to a voltage-dependent block of the channel pore by magnesium ions, preventing ion flow through it. Depolarization releases channel block and permits activated NMDA receptors to carry ionic current across the postsynaptic membrane. NMDA receptors are permeable to calcium ions as well as other ions. The NMDA receptor is modulated by a number of endogenous and exogenous compounds. Likewise, sodium, potassium and calcium ions not only pass through the NMDA receptor channel but also modulate the activity of NMDA receptors. Zinc blocks the NMDA current through NR2A-containing receptors in a noncompetitive, high affinity and voltage-independent manner. Zinc has a similar effect, but with lower potency on NR2B-containing NMDA receptors. It has also been demonstrated that polyamines do not directly activate NMDA receptors, but instead act to potentiate or inhibit glutamate-mediated responses.

Animal models of stroke and brain trauma confirm that glutamate released from affected neurons can overstimulate NMDA receptors, which in turn causes neuronal death. Therefore, compounds that block NMDA receptors have been considered candidates for treatment of stroke or head injuries. Animal studies have recently validated NMDA receptors as targets for neuroprotection in stroke, brain and spinal cord trauma, and related settings that involve brain ischemia. NMDA receptor blockers are effective in limiting the volume of damaged brain tissue in experimental models of stroke and traumatic brain injury (Choi, D. (1998), Mount Sinai J Med 65:133-138; Dirnagle et al. (1999) Tr. Neurosci. 22:391-397; Obrenovitch, T. P. and Urenjak, J. (1997) J Neurotrauma 14:677).

A number of NMDA receptor antagonists have been tested in early clinical trials for stroke. Stroke is the third leading cause of death in the United States and the most common cause of adult disability. An ischemic stroke occurs when a cerebral vessel occludes, obstructing blood flow to a portion of the brain. The only currently approved stroke therapy, tissue plasminogen activator (“TPA”), is a thrombolytic that promotes the dissolution of the thrombus within the blood vessel. Neuroprotective agents have generated as much interest as thrombolytic therapies (http://www.emedicine.com/neuro/topic488.htm, Lutsep & Clark “Neuroprotective Agents in Stroke”, Apr. 30, 2004), however, have not yet been approved for human therapy.

The most commonly studied neuroprotective agents for acute stroke block the N-methyl-D-aspartate (NMDA) receptor. Dextrorphan, an NMDA channel blocker and structural analog of a cough suppressant, was one of the first NMDA antagonists studied in human stroke patients. Unfortunately, dextrorphan caused hallucinations and agitation as well as hypotension, which limited its use (Albers et al. Stroke (1995) 26:254-258). Selfotel, a competitive NMDA antagonist, showed trends toward higher mortality within treated patients than within placebo-treated cohorts, and therefore, trials were stopped prematurely. A trial of another NMDA receptor antagonist, aptiganel HCl (Cerestat), was terminated because of concerns regarding benefit-to-risk ratios. In attempt to avoid these adverse effects, indirect NMDA receptor antagonists that work at the glycine site of the receptor were developed. These agents prevent glycine from binding, which in turn prevents glutamate from activating the receptor. Early clinical studies suggest that psychomimetic side effects occur less frequently in these glycine site NMDA antagonists. A large, 1367-patient, efficacy trial with the agent GV150526 was completed in 2000. Although the drug was reported to be safe and well tolerated, no improvement was observed in any of the 3-month outcome measures (http://www.emedicine.com/neuro/topic488.htm, Lutsep & Clark “Neuroprotective Agents in Stroke”, Apr. 30, 2004).

Epilepsy has long been considered a potential therapeutic target for glutamate receptor antagonists. NMDA receptor antagonists are known to be anti-convulsant in many experimental models of epilepsy (Bradford (1995) Progress in Neurobiology 47:477-511; McNamara, J. O. (2001) Drugs effective in the therapy of the epilepsies. In Goodman & Gliman's: The pharmacological basis of therapeutics [Eds. J. G. Hardman and L. E. Limbird] McGraw Hill, New York).

NMDA receptor antagonists may be beneficial in the treatment of chronic pain. Chronic pain, such as that due to injury of peripheral or central nerves, has often proved very difficult to treat, even with opioids. Treatment of chronic pain with ketamine and amantadine has proven beneficial, and it is believed that the analgesic effects of ketamine and amantadine are mediated by block of NMDA receptors. Several case reports have indicated that systemic administration of amantadine or ketamine substantially reduces the intensity of trauma-induced neuropathic pain. Small-scale double blind, randomized clinical trials corroborated that amantadine could significantly reduce neuropathic pain in cancer patients (Pud et al. (1998), Pain 75:349-354) and ketamine could reduce pain in patients with peripheral nerve injury (Felsby et al. (1996), Pain 64:283-291), peripheral vascular disease (Perrson et al. (1998), Acta Anaesthesiol Scand 42:750-758), or kidney donors (Stubhaug et al. (1997), Acta Anaesthesiol Scand 41:1124-1132). “Wind-up pain” produced by repeated pinpricking was also dramatically reduced. These findings suggest that central sensitization caused by nociceptive inputs can be prevented by administration of NMDA receptor anatagonists.

NMDA receptor antagonists can also be beneficial in the treatment of Parkinson's Disease (Blandini and Greenamyre (1998), Fundam Clin Pharmacol 12:4-12). The anti-Parkinsonian drug, amantadine, is an NMDA receptor channel blocker (Blanpied et al. (1997), J Neurophys 77:309-323). Amantadine is seldom used alone due to limited efficacy. However, a small-scale clinical trial demonstrated the value of amantadine as add-on therapy with L-DOPA. Amantadine reduced the severity of dyskinesias by 60% in these patients without reducing the antiparkinsonian effect of L-DOPA itself (Verhagen Metman et al. (1998), Neurology 50:1323-1326). Likewise, another NMDA receptor antagonist, CP-101,606, potentiated the relief of Parkinson's symptoms by L-DOPA in a monkey model (Steece-Collier et al., (2000) Exper. Neurol., 163:239-243).

NMDA receptor antagonists may in addition be beneficial in the treatment of brain cancers. Rapidly-growing brain gliomas can kill adjacent neurons by secreting glutamate and overactivating NMDA receptors such that the dying neurons make room for the growing tumor, and may release cellular components that stimulate tumor growth. Studies show NMDA receptor antagonists can reduce the rate of tumor growth in vivo as well as in some in vitro models (Takano, T., et al. (2001), Nature Medicine 7:1010-1015; Rothstein, J. D. and Bren, H. (2001) Nature Medicine 7:994-995; Rzeski, W., et al. (2001), Proc. Nat'l Acad. Sci 98:6372).

While NMDA-receptor antagonists might be useful to treat a number of very challenging disorders, to date, dose-limiting side effects have thus far prevented clinical use of NMDA receptor antagonists for these conditions. The first three generations of NMDA receptor antagonists (channel blockers, competitive blockers of the glutamate or glycine agonist sites, and noncompetitive allosteric antagonists) have not proved useful clinically due to toxic side effects, such as psychotic symptoms and cardiovascular effects. In addition, undesirable effects on memory and attention can also result from administration of NMDA antagonists. Further, NMDA receptor antagonists such as ketamine can also produce a psychotic state in humans reminiscent of schizophrenic symptoms (Krystal et al. (1994), Arch Gen Psychiatry 51:199-214). Additionally, ataxia, cognitive deficits, motor impairment, agitation, confusion, dizziness and hypothermia have all resulted from administration of NMDA antagonists. Thus, despite the tremendous potential for glutamate antagonists to treat many serious diseases, the severity of the side effects have caused many to abandon hope that a well-tolerated NMDA receptor antagonist could be developed (Hoyte L. et al (2004) “The Rise and Fall of NMDA Antagonists for Ischemic Stroke Current Molecular Medicine” 4(2): 131-136; Muir, K. W. and Lees, K. R. (1995) Stroke 26:503-513; Herrling, P. L., ed. (1997) “Excitatory amino acid clinical results with antagonists” Academic Press; Parsons et al. (1998) Drug News Perspective II: 523 569).

pH Sensitive NMDA Receptors

In the late 1980's, a new property of NMDA receptors was discovered and more recently exploited to develop new classes of NMDA antagonists. Two of the most prevalent subtypes of NMDA receptors have the unusual property of being normally inhibited by protons by about 50% at physiological pH (Traynelis, S. F. and Cull-Candy, S. G. (1990) Nature 345:347). The inhibition of NMDA receptors by protons is controlled by the NR2B subunit and NR2A subunit, as well as alternative exon splicing in the NR1 subunit (Traynelis et al. (1995) Science 268: 873-876; Traynelis et al. (1998), J Neurosci 18:6163-6175).

The extracellular pH is highly dynamic in mammalian brain, and influences the function of a multitude of biochemical processes and proteins, including glutamate receptor function. The pH-sensitivity of the NMDA receptor has received increasing attention for at least two reasons. First, the IC₅₀ value for proton inhibition of pH 7.4 places the receptor under tonic inhibition at physiological pH. Second, pH changes are extensively documented in the CNS during synaptic transmission, glutamate receptor activation, glutamate receptor uptake, and more prominantly during ischemia and seizures (Siesjo, BK (1985), Progr Brain Res 63:121-154; Chesler, M (1990), Prog Neurobiol 34:401-427; Chesler and Kaila (1992), Trends Neurosci 15:396-402; Amato et al. (1994), J Neurophysiol 72:1686-1696). The acidification associated with these latter pathological situations can partially inhibit NMDA receptors, which provides negative feedback that reduces their contribution to neurotoxicity (Kaku et al. (1993), Science 260:1516-1518; Munir and McGonigle (1995), J Neurosci 15:7847-7860; Vornov et al. (1996), J Neurochem 67:2379-2389; Gray et al. (1997), J Neurosurg Anesthesiol 9:180-187; but see O'Donnell and Bickler (1994), Stroke 25:171-177; reviewed by Tombaugh and Sapolsky (1993), J Neurochem 61:793-803) and seizure maintenance (Balestrino and Somjen (1988), J Physiol (Lond) 396:247-266; Velisek et al. (1994), Exp Brain Res 101:44-52). The pH sensitivity of glutamate transporters increases the likelihood that extracellular glutamate levels will be high during a period of acidification (Billups and Attwell (1996), Nature (Lond) 379:171-173), which enhances the opportunity for post-insult treatment of, for example, stroke with NMDA receptor antagonists (Tombaugh and Sapolsky (1993), J Neurochem 61:793-803).

During stroke, transient ischemia leads to a dramatic drop of pH to 6.4-6.5 in the core region of the infarct, with a modest drop in regions surrounding the core. The penumbral region, which surrounds the core and extends outward, suffers significant neuronal loss. The pH in this region drops to around pH 6.9. The pH-induced drops are exaggerated in presence of excess glutamate, and attenuated in hypoglycemic condition (see, for example, Mutch & Hansen (1984) J Cereb Blood Flow Metab 4: 17-27, Smith et al. (1986) J Cereb Blood Flow Metab 6: 574-583; Nedergaard et al. (1991) Am J Physiol 260(Pt3): R581-588; Katsura et al (1992a) Euro J Neursci 4: 166-176; and Katsura & Siesjo (1998) “Acid base metabolism in ischemia” in pH and Brain function (Eds Kaila & Ransom) Wiley-Liss, New York).

In addition to ischemia, there are various additional examples of situations in which pH changes under normal and abnormal conditions that are amenable to treatment with an NMDA antagonist. In general, tissue extracellular pH is typically more acidic than cerebrospinal fluid due to regulation of protons as well as active and passive movement of metabolites. Dynamic activity-dependent multiphasic acid and alkaline changes in extracellular pH have been known to occur for almost two decades. These changes have been described in a wide range of preparations and brain regions. They involve multiple molecular mechanisms, which include metabolic changes, lactic acid secretion, bicarbonate efflux through anionic channels, Na+/H+ and Ca2+/H+ exchange, and proton release from acidified vesicles. They are dependent on extracellular buffering systems, which in the mammalian brain largely relies on bicarbonate. Hence, the magnitude of pH changes observed often depends on the ability of CNS tissue to interconvert bicarbonate-CO2 rapidly. The enzyme that does this (carbonic anhydrase) is thus instrumental in setting the level of pH change that is achievable.

Neuropathic pain is associated with pH changes in the spinal cord. For example, single electrical stimulation of isolated spinal cord from rat pups produce an alkaline shift of 0.05 pH units, and a 0.1 pH unit shift following 10 Hz stimulation. An acidification followed the cessation of stimuli, and this acidification is larger in older animals (Jendelova & Sykova (1991) Glia 4: 56-63). In addition, 30-40 Hz stimulation of the dorsal root in frog produced in vivo a transient extracellular acidification reaching a maximum ceiling of 0.25 pH unit reduction in the lower dorsal horn. Extracellular pH changes increased with stimulus intensity and frequency (Chvatal et al. (1988) Physiol Bohemoslov 37: 203-212). Further, high frequency (10-100 Hz) nerve stimulation in adult rat spinal cord in vivo produced triphasic alkaline-acid-alkaline shifts in extracellular pH (Sykova et al. (1992) Can J Physiol Pharmacol 70: Suppl S301-309). Additionally, it has been shown that acute nociceptive stimuli (pinch, press, heat) applied to the rat hindpaw produced transient acidification of 0.01-0.05 pH units in the lower dorsal horn in vivo (laminae III-VII). Chemical or thermal peripheral injury produced prolonged 2 hour decreases in interstitial pH of 0.05-0.1 pH units. High frequency nerve stimulation produced an alkaline pH shift followed by a dominating 0.2 pH unit acid shift (Sykova & Svoboda (1990) Brain Res 512: 181-189). Thus, increased firing of pain fibers can cause a decrease in pH (acidification) of the dorsal horn of the spinal cord. This acidification could lead to an increased potency of pH dependent blockers in the region, making them useful in treatment of chonic nerve injury or chronic pain syndromes.

Subthalamic neurons are overactive in Parkinson's disease and this may result in a lower local pH. Such a reduced pH would increase potency of pH-sensitive antagonists in this region. There is a correlation in brain regions between neuronal activity and extracellular pH, with activity causing acidification. High frequency stimulation of brain slices gives an initial acidification followed by an alkalinization, followed by a slow acidification (See, for example, Chesler (1990) Prog Neurobiol 34: 401-427, Chesler & Kaila (1992) Tr Neurosci 15: 396-402, and Kaila & Chesler (1998) “Activity evoked changes in extracellular pH” in pH and Brain function (eds Kaila and Ransom). Wiley-Liss, New York).

Acidification also occurs during seizures. NMDA antagonists are anticonvulsant, and thus epilepsy represents a target in which pH sensitive NMDA antagonists could effectively act as anticonvulsants while remaining inactive outside the spatial and temporal confines of the seizure. Electrographic seizures in a wide range of preparations have been shown to cause a change in extracellular pH. For example, up to a 0.2-0.36 drop in pH can occur in cat fascia dentata or rat hippocampal CA1 or dentate during an electrically or chemically evoked seizure. Deeper drops in pH approaching 0.5 can occur under hypoxic conditions. This is a well accepted finding, being replicated in a number of preparations and laboratories (Siesjo et al (1985) J Cereb Blood Flow Metab 5: 47-57; Balestrino & Somjen (1988) J Physiol 396: 247-266; and Xiong & Stringer (2000) J Neurophysiol 83: 3519-3524).

In addition, other types of brain injury can result in acidification. “Spreading depression” is a term used to describe a slowly moving wave of electrical inactivity that occurs following a number of traumatic insults to brain tissue. Spreading depression can occur during a concussion or migraine. Acidic pH changes occur with spreading depression. Systemic alkalosis can occur with reduction in overall carbon dioxide content (hypocapnia) through, for example, hyperventilation. Conversely, systemic acidosis can occur with an increase in blood carbon dioxide (hypercapnia) during respiratory distress or conditions that impair gas exchange or lung function. Diabetic ketoacidosis and lactic acidosis represent three of the most serious acute complications of diabetes and can result in brain acidification. Further, fetal asphyxia during parturition occurs in 25 per 1000 births at term. It involves hypoxia and brain damage that is similar but not identical to ischemia.

Until 1995, it was not known whether the proton-sensitive property of the NMDA receptor could be exploited as a target for small molecule modulation of the receptor to develop therapeutics. Traynelis et al. (1995 Science 268:873) reported for the first time that the small molecule spermine could modulate NMDA receptor function through relief of proton inhibition. Spermine, a polyamine, shifts the pKa of the proton sensor to acidic values, reducing the degree of tonic inhibition at physiological pH, which appears as a potentiation of function (Traynelis et al. (1995), Science 268:873-876; Kumamoto, E (1996), Magnes Res 9(4):317-327).

In 1998, it was determined that the mechanism of action of the phenylethanolamine NMDA antagonists involved the proton sensor. Ifenprodil and CP-101,606 increased the sensitivity of the receptor to protons, thereby enhancing the proton inhibition. By shifting the pKa for proton block of NMDA receptors to more alkaline values, ifenprodil binding causes a larger fraction of receptors to be protonated at physiological pH and, thus, inhibited. In addition, ifenprodil was found to be more potent at lower pH (6.5) than higher pH (7.5) as tested in an in vitro model of NMDA-induced excitotoxicity in primary cultures of rat cerebral cortex. The authors speculated that context-dependent blockers could be created that would be inactive at physiological pH, but active at lower pH values that occur during ischemia, for use in the treatment of stroke (Mott et al. 1998 Nature Neuroscience 1:659).

Ifenprodil is neuroprotective in animal models of focal cerebral ischemia (Gotti et al. (1988), J Pharmacol Exp Ther 247:1211-1221; Dogan et al. (1997), J Neurosurg 87(6):921-926). Ifenprodil has been shown to be neuroprotective in mammals after middle cerebral artery occlusion. Dogan et al. reported a 22% decrease in infarct volume in rats, whereas Gotti et al. reported a 42% decrease infarct volume at the highest dose tested in cats. Gotti et al. also reported that SL 82.0715, an ifenprodil derivative, produced a 36-48% decrease in infarct volume at the highest dose tested in cats and rats. Unfortunately, ifenprodil and several of its analogs, including eliprodil and haloperidol (Lynch and Gallagher (1996), J Pharmacol Exp Ther 279:154-161; Brimecombe et al. (1998), J Pharmacol Exp Ther 286(2):627-634), block certain serotonin receptors and calcium channels in addition to NMDA receptors, limiting their clinical usefulness (Fletcher et al. (1995), Br J Pharmacol 116(7):2791-2800; McCool and Lovinger (1995), Neuropharmacology 34:621-629; Barann et al. (1998), Naunyn Schmiedebergs Arch Pharmacol 358:145-152). In addition, eliprodil, an ifenprodil analog, lengthens cardiac repolarisation by inhibition of IKr (Lengyel et al. (2004) Br J Pharmacol 143: 152-8), and ifenprodil and certain analogs can also inhibit calcium channels (Biton et al. (1994) Eur J Pharmacol 257:297-301; Biton et al. (1995), Eur J Pharmacol 294:91-100; Bath et al (1996), Eur J Pharmacol 299:103-112). Several more NMDA receptor-selective derivatives of ifenprodil are being considered for clinical development, including CP101,606 (Menniti et al. (1997), Eur J Pharmacol 331:117-126), Ro 25-6981 (Fischer et al. (1997), J Pharmacol Exp Ther 283:1285-1292) and Ro 8-4304 (Kew et al. (1998), Br J Pharmacol 123:463-472).

In addition to these allosteric modulators, other NMDA antagonists have been shown to produce neuroprotective effects in animal models of focal ischemia (Gill et al (1994) Cerebrovascular and Brain Metabolism Reviews 6: 225-256). These NMDA antagonists fall into three functional classes: competitive blockers of the glutamate binding site, competitive blockers of the glycine binding site and channel blockers, which produce toxic side effects or exhibit limited efficacy in humans.

(i) The competitive NMDA antagonists of the glutamate site, such as, selfotel, D-CPPene (SDZ EAA 494) and AR-R15896AR (ARL 15896AR), cause toxic side effects including agitation, hallucination, confusion and stupor (Davis et al. (2000), Stroke 31(2):347-354; Diener et al. (2002), J Neurol 249(5):561-568); paranoia and delirium (Grotta et al. (1995), J Intern Med 237:89-94); psychotomimetic-like symptoms (Loscher et al. (1998), Neurosci Lett 240(1):33-36); poor therapeutic ratio (Dawson et al. (2001), Brain Res 892(2):344-350); amphetamine-like stereotyped behaviors (Potschka et al. (1999), Eur J Pharmacol 374(2):175-187).

(ii) The glycine site antagonists, such as HA-966, L-701,324, d-cycloserine, CGP-40116, and ACEA 1021 produce toxic side effects, including significant memory impairment and motor impairment (Wlaz, P (1998), Brain Res Bull 46(6):535-540).

(iii) The NMDA receptor channel blockers, including MK-801 and ketamine, can produce toxic side effects, such as psychosis-like effects (Hoffman, D C (1992), J Neural Transm Gen Sect 89:1-10); cognitive deficits (decrements in free recall, recognition memory, and attention; Malhotra et al (1996), Neuropsychopharmacology 14:301-307); schizophrenia-like symptoms (Krystal et al (1994), Arch Gen Psychiatry 51:199-214; Lahti et al. (2001), Neuropsychopharmacology 25:455-467).

WO 02/072542 to Emory University describes a class of pH-dependent NMDA receptor antagonists that exhibit pH sensitivity tested in vitro using an oocyte assay and in an experimental model of epilepsy. However, the in vitro data using Xenopus oocytes was subject to wide variations in measured IC₅₀'s for selected compounds, which limited accurate selection of the optimal, or lead, compound. Also, since the assays were limited to cell-based screens, they lacked the ability to assess whether there is a sufficiently large drop in pH in affected ischemic tissue in vivo to observe a substantial effect caused by the pH-dependent antagonist. Further, because ischemia is peculiarly an in vivo tissue-based disease with core and penumbral damage, one did not know how far outside the core the pH dependant NMDA antagonist would be effective, given that the pH drop decreases radially from the core of the infarct. Finally, given that NMDA receptor antagonists are known to induce psychosis and other consciousness-altering side effects, it was not known whether the enhanced neuroprotective activity caused by the focal ischemic pH drop was sufficient to both observe the palliative effect of the pH-sensitive NMDA receptor antagonist and avoid the NMDA-receptor associated side effects.

In summary, to select appropriate NMDA receptor antagonists that can be tolerated in humans, the drug must not significantly affect normal functioning of glutamate neurotransmission, yet provide an effective blockade of the glutamate system during pathological conditions thereby avoiding the toxic side effects. Further, although it has been speculated since the early 1990s that pH-dependant NMDA receptor antagonists may achieve this goal, to date, this concept has not been tested in an in vivo model of ischemic injury. It has not been possible to predict whether or not pH-dependent selective NMDA antagonists that demonstrate a greater affinity for the NMDA receptor at a lower pH in vitro would also display a sufficient response in vivo to provide a commercial drug. While pH-dependant NMDA receptor anatagonists have been developed, the appropriate properties of these drugs have not yet been determined to accurately establish successful parameters for selection of a drug for human clinical use.

It is therefore an object of the invention to provide an improved or more precise method for the selection of pH dependent drugs to be used before, during or after a pH-lowering event in vivo to minimize or prevent tissue damage.

It is another object of the present invention to provide a method to identify active compounds that are useful to treat ischemic injury in vivo.

It is an object of the present invention to provide for the effective treatment of a pathogenic pH-lowering event by administration of a sufficient amount of a pH dependent compound in vivo without substantial psychotic effects.

It is an object of the present invention to provide for the effective treatment of a pathogenic pH-lowering event by administration of a sufficient amount of a pH dependent compound in vivo without substantial toxic effects.

It is a further aspect of the present invention to provide compounds and compositions that are useful to treat a pathogenic pH-lowering event in vivo.

### SUMMARY OF THE INVENTION

The inventors have established the successful parameters for selection of pH dependent compounds that bind to a glutamate receptor for improved mammalian, for example, human, medical therapy. Prior to the invention, it was not known how to rationally select a compound for in vivo use that would sufficiently protect against an in vivo destructive drop in pH, which results in deleterious in vivo effects. This invention solves the long felt need to accelerate the much needed discovery and use of effective neuroprotective agents. The dirth of present agents that accomplish this goal is a testament to the need for the invention. This has been accomplished by carrying out a careful comparison of repeated data on the pH potency boost of a candidate drug in vitro with the drug's performance in a whole animal model of ischemia. For the first time, the inventors have correlated performance in vitro with performance in vivo and established the meets and bounds of the selection criteria for the treatment or prevention of a wide variety of debilitating diseases which involve pH drops. The inventors further provide active compounds that can be used according to the process further described herein. It is believed that the inventors are the first to determine the efficacy of pH dependent glutamate receptor antagonists in vivo.

In one aspect of the present invention, a process is provided to identify a compound that is useful to treat ischemic injury in a mammal, particularly a human, by: (i) assessing the potency boost of the compound at physiological pH versus “disorder-induced low pH” (for example, IC₅₀ at physiological pH/IC₅₀ at “disorder induced low pH”) in a cell by repeating the potency boost experiment at least 5 times such that the 95% confidence interval does not change more than 15% with the addition of a new experiment; (ii) testing the compound in an animal model of transient focal ischemia and measuring the effect of the compound on the infarct volume by repeating the experiment at least 12 times such that the 95% confidence interval does not change more than 5% with the addition of a new experiment; (iii) selecting a compound that has a potency boost of at least 5 according to step (i) and at least a 30% decrease in infarct volume according to step (ii). According to the invention, a candidate drug must meet or exceed both the in vitro and in vivo criteria to be an effective drug for human use. In one embodiment, the potency boost can be determined in a cell that expresses a glutamate receptor. In another embodiment, the potency boost can be determined in a cell that expresses an NMDA, AMPA and/or kainate receptor. In one embodiment, the cell can express an NR1 subunit and at least one NR2 subunit of an NMDA receptor. In a further embodiment, the NR2 subunit can be the NR2B subunit. In another embodiment, the NR2 subunit can be the NR2A subunit.

In another more general aspect of the present invention, a process is provided wherein a compound is selected to treat a disorder that lowers the pH in a manner that activates an NMDA receptor antagonist that (i) exhibits a potency boost of at least 5 as determined in experiments in which the potency boost of the compound is assessed at physiological pH versus “disorder-induced low pH” (for example, IC₅₀ at physiological pH/IC₅₀ at “disorder induced low pH”) as tested in a cell by repeating the potency boost experiments at least 5 times such that the 95% confidence interval does not change more than 15% with the addition of a new experiment and (ii) exhibits at least a 30% decrease in infarct volume as measured in an animal model of focal ischemia as determined by repeating the experiment at least 12 times such that the 95% confidence interval does not change more than 5% with the addition of a new experiment. In one embodiment, the potency boost can be determined in a cell that expressed a glutamate receptor. In another embodiment, the potency boost can be determined in a cell that expresses an NMDA, AMPA and/or kainate receptor. In one embodiment, the cell can express an NR1 subunit and at least one NR2 subunit of an NMDA receptor. In a further embodiment, the NR2 subunit can be the NR2B subunit. In another embodiment, the NR2 subunit can be the NR2A subunit.

In one particular embodiment, a method is provided to select a compound or a compound that exhibits a potency boost of at least 5 as determined in experiments in which the potency boost of the compound is assessed at physiological pH versus “disorder-induced low pH” (for example, IC50 at phys pH/IC50 at “disorder induced low pH”) as tested in a cell expressing a NR1/NR2A NMDA receptor and/or a NR1/NR2B NMDA receptor by repeating the potency boost experiments at least five times such that the 95% confidence interval does not change more than 15% with the addition of a new experiment. In another particular embodiment, a method is provided to select a compound or a compound that exhibits at least a 30% decrease in infarct volume as measured in an animal model of focal ischemia as determined by repeating the experiment at least 12 times such that the 95% confidence interval does not change more than 5% with the addition of a new experiment. In another particular embodiment, the “disorder-induced low pH” can be associated with an ischemic disorder, such as stroke.

FIG. 1 is illustrative of the novel parameters for selection of an NMDA-receptor antagonist, for which improved mammalian, for example, human, medical therapy can be achieved.

In one embodiment, the compound selected according to the processes and methods described herein is a selective NR1/NR2A NMDA receptor and/or a NR1/NR2B NMDA receptor antagonist. In one embodiment, the compound is not an NMDA receptor channel blocker. In another embodiment, the compound selected according to the processes and methods described herein is not an NMDA receptor glutamate site antagonist. In another embodiment, the compound selected according to the processes and methods described herein is not an NMDA receptor glycine site antagonist.

In an additional embodiment, the compound does not exhibit substantial toxic side effects, such as, for example, motor impairment or cognitive impairment. Additionally or alternatively, the compound has a therapeutic index equal to or greater than at least 2. In a further additional or alternative embodiment, the compound is at least 10 times more selective for binding to an NMDA receptor than any other glutamate receptor. In one embodiment, oocyte cells are used to determine the potency boost. In another embodiment, the middle cerebral artery occlusion model is used as the animal model of transient focal ischemia, for example, in rodents, such as mice.

In further embodiments, the compound exhibits a potency boost of at least 6, 7, 8, 9, 10, 15 or 20 according to step (i) and at least a 35%, 40%, 45%, 50%, 55%, or 60% decrease in infarct volume according to step (ii). In certain embodiments of the present invention, the mean, i.e. the sum of all the observations divided by the number of observations, can be calculated for the potency boost and infarct volume experiments and the mean value of the compound can exhibit a potency boost of at least 5 at physiological pH versus ischemic pH (i.e., (IC50 at phys pH/IC50 at Isc pH)) and at least a 30% decrease in infarct volume, such as illustrated in FIG. 1.

In further aspects of the present invention the compound selected according to the processes and methods described herein can be:

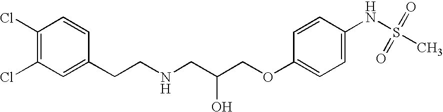

as well as pharmaceutically acceptable salts, esters, enantiomers, enantiomeric mixtures, and mixtures.

In another embodiment, the compound selected according to the processes and methods described herein can be:

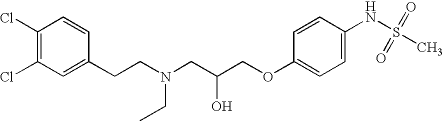

as well as pharmaceutically acceptable salts, esters, enantiomers, enantiomeric mixtures, and mixtures.

In another embodiment, the compound selected according to the processes and methods described herein can be:

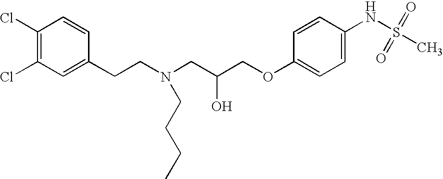

as well as pharmaceutically acceptable salts, esters, enantiomers, enantiomeric mixtures, and mixtures.

In a further embodiment, the compound selected according to the processes and methods described herein can be:

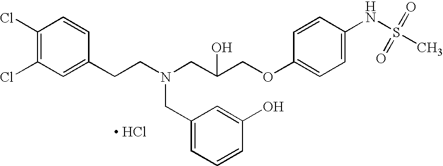

as well as pharmaceutically acceptable salts, esters, enantiomers, enantiomeric mixtures, and mixtures.

In a further embodiment, the compound selected according to the processes and methods described herein can be: 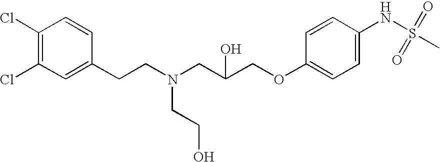
as well as pharmaceutically acceptable salts, esters, enantiomers, enantiomeric mixtures, and mixtures.

In one particular embodiment, the compounds described above can bind to the NR2B subunit of the NMDA receptor. In another particular embodiment, the compounds above can be selective for the NR2B subunit of the NMDA receptor. In one embodiment, compounds (S) 98-5, (S) 93-4, (S) 93-8, (S) 93-31 and (S) 93-41 as disclosed herein can bind to the NR2B subunit of the NMDA receptor, for example as indicated in FIG. 1. In another embodiment, compounds (S) 98-5, (S) 93-4, (S) 93-8, (S) 93-31 and (S) 93-41 as disclosed herein can be selective for the NR2B subunit of NMDA receptors.

Further provided are methods to attenuate the progression of an ischemic or excitotoxic cascade associated with a drop in pH by administering a compound selected according to the processes or methods described herein. In addition, methods are provided to decrease infarct volume associated with a drop in pH by administering a compound selected according to the processes or methods described herein. Further, a method is provided to decrease cell death associated with a drop in pH by administering a compound selected according to the processes or methods described herein. Still further, methods are provided to decrease behavioral deficits associated with an ischemic event associated with a drop in pH by administering a compound selected according to the processes or methods described herein.

In additional aspects of the present invention, methods are provided to treat patients by administering a compound selected according to the methods or processes described herein. Any disease, condition or disorder which induces a low pH can be treated according to the methods described herein.

In one embodiment, methods are provided to treat patients with ischemic injury or hypoxia, or prevent or treat the neuronal toxicity associated with ischemic injury or hypoxia, by administering a compound selected according to the methods or processes described herein. In one particular embodiment, the ischemic injury can be stroke. In another particular embodiment, the ischemic injury can be vasospasm after subarachnoid hemorrhage. In other embodiments, the ischemic injury can be selected from, but not limited to, one of the following: traumatic brain injury, cognitive deficit after bypass surgery, cognitive deficit after carotid angioplasty; and/or neonatal ischemia following hypothermic circulatory arrest.

In another embodiment, methods are provided to treat patients with neuropathic pain or related disorders by administering a compound selected according to the methods or processes described herein. In certain embodiments, the neuropathic pain or related disorder can be selected from the group including, but not limited to: peripheral diabetic neuropathy, postherpetic neuralgia, complex regional pain syndromes, peripheral neuropathies, chemotherapy-induced neuropathic pain, cancer neuropathic pain, neuropathic low back pain, HIV neuropathic pain, trigeminal neuralgia, and/or central post-stroke pain.

In another embodiment, methods are provided to treat patients with brain tumors by administering a compound selected according to the methods or processes described herein. In a further embodiment, methods are provided to treat patients with neurodegenerative diseases by administering a compound selected according to the methods or processes described herein. In one embodiment, the neurodegenerative disease can be Parkinson's disease. In another embodiment, the neurodegenerative disease can be Alzheimer's, Huntington's and/or Amyotrophic Lateral Sclerosis.

Further, compounds selected according to the methods or processes described herein can be used prophylactically to prevent or protect against such diseases or neurological conditions, such as those described herein. In one embodiment, patients with a predisposition for an ischemic event, such as a genetic predisposition, can be treated prophylactically with the methods and compounds described herein. In another embodiment, patients that exhibit vasospasms can be treated prophylactically with the methods and compounds described herein. In a further embodiment, patients that have undergone cardiac bypass surgery can be treated prophylactically with the methods and compounds described herein.

### DESCRIPTION OF THE FIGURES

FIG. 1 is an illustration of the comparison of the in vitro potency boost at pH 6.9 vs 7.6 versus tissue infarct volume reductions for a selection of NMDA receptor antagonists.

The infarct volume was measured in C57B1/6 mice following a transient or permanent focal ischemic event for compounds indicated by solid symbols. Drug was applied intracerebroventricularly (ICV; 1 microliter of 0.5 mM; solid squares) or by intraperitoneal injection (IP, solid circles; NP93-4, 30 mg/kg; NP93-5, 10-30 mg/kg, results for both doses were similar thus combined; NP93-40, 10-30 mg/kg; NP93-8, 30 mg/kg; NP93-31, 3 mg/kg) as described herein. Error bars are SEM. Infarct volume in drug-treated animals was directly measured and expressed as a percent of the infarct volume in vehicle injected control mice typically subjected to ischemia the same day. Open symbols show the reduction in infarct volume by administration of CNS1102 (CN, aptiganel or Cerestat, Dawson et al., 2001), dextromethorphan (DM, Steinberg et al., 1995), dextrorphan (DX; Steinberg et al., 1995), levomethorphan (LM; Steinberg et al., 1995), (S) ketamine (KT; Proescholdt et al., 2001), memantine (MM; Culmsee et al. 2004), ifenprodil (IF, Dawson et al. 2001), CP101,606 (CP; Yang et al. 2003), AP7 (Swan and Meldrum, 1990), Selfotel (CGS19755, Dawson et al., 2001), (R)HA966 (HA; Dawson et al., 2001), remacemide (RE, Dawson et al., 2001), haloperidol (O'Neill et al., 1998), 7-Cl-kynurenic acid (C K, Wood et al., 1992) and stereoisomer of MK801 (+MK or −MK; Dravid et al., in preparation) as described in the literature in various rodent or rabbit ischemia models. Percent reduction in infarct was calculated from the ratio of the infarct volume in drug to that in control for all compounds except ketamine and 7-Cl-kynurenic acid, for which the percent reduction in neuronal density by drug was measured. The pH boosts for ifenprodil and CP101,606 were determined from the literature (Mott et al., 1998). For all other compounds the potency boosts for the inhibition of NR1/NR2B containing NMDA receptors at pH 6.9 vs 7.6 were calculated as described herein, except competitive antagonists, which were evaluated in 2 experiments (see Table 3 below). When compounds were less potent at acidic pH, the potency boost is shown as the negative reciprocal.

The grey shadowed area indicates the area which defines the identified bounds of the criteria for effective drug performance. The drugs that fall within the bounds are those that have a mean (not error bars) within the grey blocked area. Of the 24 compounds tested, 19 compounds fall outside the area of the invention (grey shaded area), indicating that over 75% of compounds tested fail to meet the identified standard for effective in vivo therapy.

FIG. 2 is an illustration of the comparison of the in vitro potency boost of selected compounds 93-97, 93-43, 93-5, 93-41 and 93-31 at pH 6.9 vs 7.6 versus tissue infarct volume protection when the test drug was applied intracerebroventricularly (ICV; solid squares). The grey shadowed area indicates the area which defines the identified bounds of the criteria for improved drug performance. The drugs which fall within the bounds are those that have a mean (not error bars) within the grey blocked area.

FIG. 3 is an illustration of the comparison of the in vitro potency boost of selected compounds 93-4, 93-5, 93-8, 93-31, 93-40 at pH 6.9 vs 7.6 versus tissue infarct volume protection when the test drug was applied by intraperitoneal injection (IP, solid circles). The grey shadowed area indicates the area which defines the identified bounds of the criteria for improved drug performance. The drugs which fall within the bounds are those that have a mean (not error bars) within the grey blocked area.

FIG. 4 is an illustration of the comparison of the in vitro potency boost at pH 6.9 vs 7.6 versus tissue infarct volume of selected compounds. The grey shadowed area indicates the area which defines the identified bounds of the criteria for improved drug performance. The right panel shows comparison for NR1NR2A and the left panel shows comparison for NR1/NR2B.

FIG. 5 illustrates the effect of Compounds 93-31 and (+)MK-801 on locomotor activity of rats, quantified as light beam breaks counted by a computer during a 2 hour period following 1 hour habituation. The Locomotor Activity Index is the total number of beam breaks during the trial divided by 1000. Compound 93-31 had no significant effect on locomotor activity index when administered IP in doses up to 300 mg/kg, whereas (+)MK-801 induced locomotor activity at low doses and ataxia at higher doses.

FIG. 6 illustrates that the injured paw showed substantial allodynia in the animal model of neuropathic pain. Animals in the vehicle group displayed significant mechanical allodynia for the entire duration of the study. Shown are mean±SEM (n=10) von Frey thresholds in the injured and normal paws of animals treated with vehicle. The difference between paws was significant at all time points (Mann-Whitney test).

FIG. 7 ahows that Compound 93-31 (administered i.p.) showed no effect on the normal paw. Shown are the mean±SEM (n=10-12) von Frey thresholds in the normal paw in animals treated with vehicle, gabapentin or 30 and 100 mg/kg doses of Compound 93-31 administered i.p.

FIG. 8 shows that Compound 93-97 (i.p.) showed no effect on normal paw. NeurOp 93-97 did not alter von Frey thresholds in the normal paw. Shown are the mean±SEM (n=10-12) von Frey thresholds in the normal paw in animals treated with vehicle, gabapentin or 30 and 100 mg/kg doses of 93-97 administered i.p.

FIG. 9 illustrates that Compound 93-31 (100 mg/kg) administered i.p. attenuated mechanical allodynia in the Spinal Nerve Ligation (SNL) model in the rat. Treatment with the compound 93-31 (100 mg/kg i.p.) generated observable analgesia at 30 and 60 min following its administration. There was no analgesic effect of 30 mg/kg of Compounds 93-31, and 30 and 100 mg/kg of 93-97 any time point studied. Statistical analysis of the vehicle group in this study indicated there was no significant difference in von Frey threshold between baseline and at 60 120 and 240 minute time point (Friedman two-way ANOVA).

FIG. 10 shows that Compound 93-31 (100 mg/kg) administered i.p. attenuated mechanical allodynia in SNL rat. I.P. administration of Compound 93-31 test compound (100 mg/kg) reduced mechanical allodynia. Shown are the mean±SEM (n=10-12) von Frey thresholds in the injured paw of animals treated with vehicle, gabapentin (reference compound) or 30 and 100 mg/kg doses of Compound 93-31 administered i.p. Post-hoc analysis (Dunn's test) showed significant pair-wise differences between Compound 93-31 (100 mg/kg) and vehicle groups at 30 and 60 minute (p<0.01). The effect of gabapentin at 60, 120 and 240 minutes was also significant (p<0.001, p<0.01, and p<0.01 respectively).

FIG. 11 is an illustration of the comparison of the in vitro potency boost at pH 6.9 vs 7.6 versus fold increase in pain threshold in a rodent spinal nerve ligation model. Potency boosts were determined for each compound as decribed herein. The pain threshold was measured after administration of Compound 93-31. The pain threshold values were previousle reported for IF (ifenprodil, De Vry et al., Eur J Pharmacol 491:137-148, 2004), K (ketamine, Chaplan et al. JPET 280:829-838 1997), CP (CP11,606, Boyce et al. Neduropharmacol 38:611-623, 1999), MK (MK801, Chaplan et al. JPET 280:829-838 1997), D (dextrorphan, Chaplan et al. JPET 280:829-838 1997), DM (dextromethorphan, Chaplan et al. JPET 280:829-838 1997), and M (memantine, Chaplan et al. JPET 280:829-838 1997).

The grey shadowed area indicates the area which defines the identified bounds of the criteria for improved drug performance. The drugs that fall within the bounds are those that have a mean (not error bars) within the grey blocked area.

### DETAILED DESCRIPTION

The inventors have established the successful parameters for selection of an NMDA-receptor antagonist for improved mammalian, for example, human, clinical performance. This has been accomplished by carrying out a careful comparison of repeated data on the pH potency boost of a candidate drug in vitro with the drug's performance in a whole animal model of ischemia. For the first time, the inventors have correlated performance in vitro with performance in vivo and established the meets and bounds of the selection criteria for the treatment or prevention of a wide variety of debilitating diseases which involve pH drops that affect NMDA receptors. The inventors further provide active compounds that can be used according to the process further described herein. It is believed that the inventors are the first to determine the efficacy of pH dependent glutamate receptor antagonists in vivo.

In one aspect of the present invention, a process is provided to identify a compound that is useful to treat ischemic injury in a mammal, particularly a human, by: (i) assessing the potency boost of the compound at physiological pH versus “disorder-induced low pH” (for example, IC₅₀ at physiological pH/IC₅₀ at “disorder induced low pH”) in a cell by repeating the potency boost experiment at least 5 times such that the 95% confidence interval does not change more than 15% with the addition of a new experiment; (ii) testing the compound in an animal model of transient focal ischemia and measuring the effect of the compound on the infarct volume by repeating the experiment at least 12 times such that the 95% confidence interval does not change more than 5% with the addition of a new experiment; (iii) selecting a compound that has a potency boost of at least 5 according to step (i) and at least a 30% decrease in infarct volume according to step (ii). According to the invention, a candidate drug must meet or exceed both the in vitro and in vivo criteria to be an effective drug for human use. In one embodiment, the potency boost can be determined in a cell that expresses a glutamate receptor. In another embodiment, the potency boost can be determined in a cell that expresses an NMDA, AMPA and/or kainate receptor. In one embodiment, the cell can express an NR1 subunit and at least one NR2 subunit of an NMDA receptor. In a further embodiment, the NR2 subunit can be the NR2B subunit. In another embodiment, the NR2 subunit can be the NR2A subunit.

In another more general aspect of the present invention, a process is provided wherein a compound is selected to treat a disorder that lowers the pH in a manner that activates an NMDA receptor antagonist that (i) exhibits a potency boost of at least 5 as determined in experiments in which the potency boost of the compound is assessed at physiological pH versus “disorder-induced low pH” (for example, IC₅₀ at physiological pH/IC₅₀ at “disorder induced low pH”) as tested in a cell by repeating the potency boost experiments at least 5 times such that the 95% confidence interval does not change more than 15% with the addition of a new experiment and (ii) exhibits at least a 30% decrease in infarct volume as measured in an animal model of focal ischemia as determined by repeating the experiment at least 12 times such that the 95% confidence interval does not change more than 5% with the addition of a new experiment. In one embodiment, the potency boost can be determined in a cell that expressed a glutamate receptor. In another embodiment, the potency boost can be determined in a cell that expresses an NMDA, AMPA and/or kainate receptor. In one embodiment, the cell can express an NR1 subunit and at least one NR2 subunit of an NMDA receptor. In a further embodiment, the NR2 subunit can be the NR2B subunit. In another embodiment, the NR2 subunit can be the NR2A subunit.

In another more general aspect of the present invention, a process is provided wherein a compound to treat a disorder that lowers the pH in a manner that activates an NMDA receptor antagonist is selected that (i) exhibits a potency boost of at least 5 as determined in experiments in which the potency boost of the compound is assessed at physiological pH versus “disorder-induced low pH” (for example, IC₅₀ at physiological pH/IC₅₀ at “disorder induced low pH”) is tested in a cell by repeating the potency boost experiments at least 5 times such that the 95% confidence interval does not change more than 15% with the addition of a new experiment and (ii) exhibits at least a 30% decrease in infarct volume as measured in an animal model of focal ischemia as determined by repeating the experiment at least 12 times such that the 95% confidence interval does not change more than 5% with the addition of a new experiment. In one embodiment, the potency boost can be determined is a cell that expressed a glutamate receptor. In another embodiment, the potency boost can be determined in a cell that expresses an NMDA, AMPA, and/or kainate receptor. In one embodiment, the cell can express an NR1 subunit and at least one NR2 subunit of an NMDA receptor. In a further embodiment, the NR2 subunit can be the NR2B subunit. In another embodiment, the NR2 subunit can be the NR2A subunit.

In one particular embodiment, a method is provided to select a compound or a compound is selected that exhibits a potency boost of at least 5 as determined in experiments in which the potency boost of the compound is assessed at physiological pH versus “disorder-induced low pH” (for example, IC50 at phys pH/IC50 at “disorder induced low pH”) as tested in a cell expressing a NR1/NR2A NMDA receptor and/or a NR1/NR2B NMDA receptor by repeating the potency boost experiments at least five times such that the 95% confidence interval does not change more than 15% with the addition of a new experiment. In another particular embodiment, a method is provided to select a compound or a compound is selected that exhibits at least a 30% decrease in infarct volume as measured in an animal model of focal ischemia as determined by repeating the experiment at least 12 times such that the 95% confidence interval does not change more than 5% with the addition of a new experiment. In another particular embodiment, the “disorder-induced low pH” can be associated with an ischemic disorder, such as stroke.

Further provided are methods to attenuate the progression of an ischemic or excitotoxic cascade associated with a drop in pH by administering a compound selected according to the processes or methods described herein. In addition, methods are provided to decrease infarct volume associated with a drop in pH by administering a compound selected according to the processes or methods described herein. Further, a method is provided to decrease cell death associated with a drop in pH by administering a compound selected according to the processes or methods described herein. Still further, methods are provided to decrease behavioral deficits associated with an ischemic event associated with a drop in pH by administering a compound selected according to the processes or methods described herein. In other embodiments, non-behavioral side effects can also be reduced, for example, vaculozation.

I. Assessment of the Potency Boost

The term “oocyte” describes the mature animal ovum which is the final product of oogenesis and also the precursor forms being the oogonium, the primary oocyte and the secondary oocyte respectively.

“Transfection” refers to the introduction of DNA into a host cell. Cells do not naturally take up DNA. Thus, a variety of technical “tricks” are utilized to facilitate gene transfer. Numerous methods of transfection are known to the ordinarily skilled artisan, for example, CaPO₄, electroporation and/or direct microinjection of DNA or RNA directly into the cell (J. Sambrook, E. Fritsch, T. Maniatis, Molecular Cloning: A Laboratory Manual, Cold Spring Laboratory Press, 1989). Transformation of the host cell is the indicia of successful transfection.

Expression of Glutamate Receptors in Cells

In one aspect of the present invention, the potency boost of a compound can be determined in cells expressing glutamate receptors. In one embodiment, the cells can endogenously express glutamate receptors. In one embodiment, the cells can express NMDA receptors. In another embodiment, the cells can express AMPA receptors. In a further embodiment, the cells can express kainate receptors. In a still further embodiment, the cells can express orphan glutamate receptors. In another embodiment, the cells can endogenously express NMDA receptors. Cells that can endogenously express NMDA receptors, include, but are not limited to: stem cells, P19 cells, neuroepithelial cells, neuroendothelial cells, dopaminergic substantia nigra neurons, astrocytes, magnocellular neuroendocrine cells, supraoptic neurons, cerebellar neurons, brain stem cells, diencephalic neurons, midbrain neurons, hindbrain neurons, spinal cord motor neurons, spinal cord interneurons, dorsal horn neurons, cortical neurons, cerebellar granule cells, hippocampal neurons, septum neurons, caudate cells, putaman cells, striatal cells, olfactory bulb cells, thalamic cells, CA1 pyramidal cells, basal ganglia cells, layer IV neurons of rat visual cortex, somatosensory cortical neurons, and pancreatic cells.

In another embodiment, the cell can be genetically modified to express glutamate receptors. In one particular embodiment, oocyte cells can be genetically modified to express glutamate receptors. Any suitable oocyte can be used as known by one skilled in the art, including, but not limited to frog oocytes, such as Xenopus oocytes, which include, but are not limited to Xenopus laevis, Xenopus tropicalis, Xenopus muelleri, Xenopus wittei, Xenopus gilli, and Xenopus borealis. In one embodiment, the oocytes can be isolated from the ovaries of the animal according to any technique known to one skilled in the art.

In other embodiments, any suitable cell type, including primary cell lines, can be genetically modified to express glutamate receptors, including, but not limited to: Chinese hamster ovary (CHO) cells, HEK kidney cells, bacterial cells, E. Coli cells, yeast cells, neuronal cells, heart cells, lung cells, stomach cells, spleen cells, pancreas cells, kidney cells, liver cells, intestinal cells, skin cells, hair cells, hypothalamic cells, pituitary cells, epithelial cells, fibroblast cells, neural cells, keratinocytes, hematopoietic cells, melanocytes, chondrocytes, lymphocytes (B and T), macrophages, monocytes, mononuclear cells, cardiac muscle cells, other muscle cells, cumulus cells, epidermal cells, endothelial cells, Islets of Langerhans cells, blood cells, blood precursor cells, bone cells, bone precursor cells, neuronal stem cells, primordial stem cells, hepatocytes, keratinocytes, umbilical vein endothelial cells, aortic endothelial cells, microvascular endothelial cells, fibroblasts, liver stellate cells, aortic smooth muscle cells, cardiac myocytes, neurons, Kupffer cells, smooth muscle cells, Schwann cells, and epithelial cells, erythrocytes, platelets, neutrophils, lymphocytes, monocytes, eosinophils, basophils, adipocytes, chondrocytes, pancreatic islet cells, thyroid cells, parathyroid cells, parotid cells, tumor cells, glial cells, astrocytes, red blood cells, white blood cells, macrophages, epithelial cells, somatic cells, pituitary cells, adrenal cells, hair cells, bladder cells, kidney cells, retinal cells, rod cells, cone cells, heart cells, pacemaker cells, spleen cells, antigen presenting cells, memory cells, T cells, B cells, plasma cells, muscle cells, ovarian cells, uterine cells, prostate cells, vaginal epithelial cells, sperm cells, testicular cells, germ cells, egg cells, leydig cells, peritubular cells, sertoli cells, lutein cells, cervical cells, endometrial cells, mammary cells, follicle cells, mucous cells, ciliated cells, nonkeratinized epithelial cells, keratinized epithelial cells, lung cells, goblet cells, columnar epithelial cells, squamous epithelial cells, embryonic stem cells, osteocytes, osteoblasts, and osteoclasts.

In one embodiment, the cell can be genetically modified to express selected AMPA receptor subunits. The AMPA receptor subunit can be a GluR1, GluR2, GluR3, or GluR4 subunit or any combination thereof. AMPA receptors are commonly known to one skilled in the art. In another the cell can be genetically modified to express selected kainate receptor subunits. The kainate receptor subunit can be a GluR5, GluR6, GluR7, KA1, or KA2 subunit or any combination thereof. Kainate receptors are commonly known to one skilled in the art. In a further embodiment, the cell can be genetically modified to express selected orphan glutamate receptor subunits. The orphan gluitamatereceptor subunit can be a delta-1 or delta-2 subunit, or and combination thereof, such receptors are known to one skilled in the art.

In another embodiment, the cell can be genetically modified to express selected NMDA receptor subunits. NMDA receptors are composed of NR1, NR2 (A, B, C, and D), and NR3 (A and B) subunits, which determine the functional properties of native NMDA receptors. NMDA receptors are heteromeric proteins composed of NR1 with NR2 and/or NR3 subunits. DNA encoding any of the NMDA receptor subunits from any species can be used to genetically modify the cells. Table A provides the GenEMBL Accession numbers for NMDA receptor subunits.

TABLE A

| NMDA Receptor Subunit | Species: GenEMBL Accession Number |
|---|---|
| NMDA NR1 | Mouse: D10028 |
| | Rat: X63255 |
| | Human: X58633 |
| NMDA NR2A | Mouse: D10217 |
| | Rat: D13211 |
| | Human: U09002 |
| NMDA NR2B | Mouse: D10651′ |
| | Rat: M91562 |
| | Human: U28861a |
| NMDA NR2C | Mouse: D10694 |
| | Rat: D13212 |
| | Human: BC059384 |
| NMDA NR2D | Mouse: D12822 |
| | Rat: D13214 |
| | Human: U77783 |
| NMDA NR3A | Human: AF416558 |
| | Rat: L34938 |
| NMDA NR3B | Rat: NM_133308 |
| | Mouse: NM_130455 |
| | Human: NM_138690 |

The cRNA, for example, can be synthesized from the cDNA template and then injected into the cell. Alternatively, the cDNA encoding the receptor subunit can be inserted into a construct or vector prior to insertion into the cell. Techniques which can be used to allow the DNA construct or vector entry into the host cell include calcium phosphate/DNA co-precipitation, microinjection of DNA into the nucleus, electroporation, bacterial protoplast fusion with intact cells, transfection, or any other technique known by one skilled in the art. The DNA can be linear or circular, relaxed or supercoiled DNA. For various techniques for transfecting mammalian cells, see, for example, Keown et al., Methods in Enzymology Vol. 185, pp. 527-537 (1990).

The construct or vector can be prepared in accordance with methods known in the art. The construct can be prepared using a bacterial vector, including a prokaryotic replication system, e.g. an origin recognizable by E. coli, at each stage the construct can be cloned and analyzed. A selectable marker can also be employed. Once the vector containing the construct has been completed, it can be further manipulated, such as by deletion of the bacterial sequences, linearization, introducing a short deletion in the homologous sequence. After final manipulation, the construct can be introduced into the cell.

The present invention further includes recombinant constructs comprising one or more of the sequences as described above. The constructs can be in the form of a vector, such as a plasmid or viral vector, into which a sequence of the invention can been inserted, in a forward or reverse orientation. The construct can also include regulatory sequences, including, for example, a promoter, operably linked to the sequence. Large numbers of suitable vectors and promoters are known to those of skill in the art, and are commercially available. The following vectors are provided by way of example: pBs, pQE-9 (Qiagen), phagescript, PsiX174, pBluescript SK, pBsKS, pBSSK, pGEM, pNH8a, pNH16a, pNH18a, pNH46a (Stratagene); pTrc99A, pKK223-3, pKK233-3, pDR540, pRIT5 (Pharmacia). Eukaryotic: pCiNeo, pWLneo, pSv2cat, pOG44, pXT1, pSG (Stratagene) pSVK3, pBPv, pMSG, pSVL (Pharmiacia). Also, any other plasmids and vectors can be used as long as they are replicable and viable in the host. Vectors known in the art and those commercially available (and variants or derivatives thereof) can be used in accordance with the invention be engineered to include one or more recombination sites for use in the methods of the invention. Such vectors can be obtained from, for example, Vector Laboratories Inc., Invitrogen, Promega, Novagen, NEB, Clontech, Boehringer Mannheim, Pharmacia, EpiCenter, OriGenes Technologies Inc., Stratagene, PerkinElmer, Pharmingen, and Research Genetics. Other vectors of interest include eukaryotic expression vectors such as pFastBac, pFastBacHT, pFastBacDUAL, pSFV, and pTet-Splice (Invitrogen), pEUK-C1, pPUR, pMAM, pMAMneo, pBI101, pBI121, pDR2, pCMVEBNA, and pYACneo (Clontech), pSVK3, pSVL, pMSG, pCH110, and pKK232-8 (Pharmacia, Inc.), p3'SS, pXT1, pSG5, pPbac, pMbac, pMC1neo, and pOG44 (Stratagene, Inc.), and pYES2, pAC360, pBlueBacHis A, B, and C, pVL1392, pBlueBacIII, pCDM8, pcDNA1, pZeoSV, pcDNA3 pREP4, pCEP4, and pEBVHis (Invitrogen, Corp.) and variants or derivatives thereof.

Additional vectors suitable for use in the invention include pUC18, pUC19, pBlueScript, pSPORT, cosmids, phagemids, YAC's (yeast artificial chromosomes), BAC's (bacterial artificial chromosomes), P1 (Escherichia coli phage), pQE70, pQE60, pQE9 (quagan), pBS vectors, PhageScript vectors, BlueScript vectors, pNH8A, pNH16A, pNH18A, pNH46A (Stratagene), pcDNA3 (Invitrogen), pGEX, pTrsfus, pTrc99A, pET-5, pET-9, pKK223-3, pKK233-3, pDR540, pRIT5 (Pharmacia), pSPORT1, pSPORT2, pCMVSPORT2.0 and pSV-SPORT1 (Invitrogen) and variants or derivatives thereof. Viral vectors can also be used, such as lentiviral vectors (see, for example, WO 03/059923; Tiscornia et al. PNAS 100:1844-1848 (2003)). Additional vectors of interest include pTrxFus, pThioHis, pLEX, pTrcHis, pTrcHis2, pRSET, pBlueBacHis2, pcDNA3.1/His, pcDNA3.1(−)/Myc-His, pSecTag, pEBVHis, pPIC9K, pPIC3.5K, pAO815, pPICZ, pPICZA, pPICZB, pPICZC, pGAPZA, pGAPZB, pGAPZC, pBlueBac4.5, pBlueBacHis2, pMelBac, pSinRep5, pSinHis, pIND, pIND(SP1), pVgRXR, pcDNA2.1, pYES2, pZErO1.1, pZErO-2.1, pCR-Blunt, pSE280, pSE380, pSE420, pVL1392, pVL1393, pCDM8, pcDNA1.1, pcDNA1.1/Amp, pcDNA3.1, pcDNA3.1/Zeo, pSe, SV2, pRc/CMV2, pRc/RSV, pREP4, pREP7, pREP8, pREP9, pREP 10, pCEP4, pEBVHis, pCR3.1, pCR2.1, pCR3.1-Uni, and pCRBac from Invitrogen; λ ExCell, λ gt11, pTrc99A, pKK223-3, pGEX-1 λ T, pGEX-2T, pGEX-2TK, pGEX-4T-1, pGEX-4T-2, pGEX-4T-3, pGEX-3X, pGEX-5X-1, pGEX-5X-2, pGEX-5X-3, pEZZ18, pRIT2T, pMC1871, pSVK3, pSVL, pMSG, pCH110, pKK232-8, pSL1180, pNEO, and pUC4K from Pharmacia; pSCREEN-1b(+), pT7Blue(R), pT7Blue-2, pCITE-4abc(+), pOCUS-2, pTAg, pET-32LIC, pET-30LIC, pBAC-2 cp LIC, pBACgus-2 cp LIC, pT7Blue-2 LIC, pT7Blue-2, λ SCREEN-1, λ BlueSTAR, pET-3abcd, pET-7abc, pET9abcd, pET11abcd, pET12abc, pET-14b, pET-15b, pET-16b, pET-17b-pET-17xb, pET-19b, pET-20b(+), pET-21abcd(+), pET-22b(+), pET-23abcd(+), pET-24abcd(+), pET-25b(+), pET-26b(+), pET-27b(+), pET-28abc(+), pET-29abc(+), pET-30abc(+), pET-31b(+), pET-32abc(+), pET-33b(+), pBAC-1, pBACgus-1, pBAC4x-1, pBACgus4x-1, pBAC-3 cp, pBACgus-2 cp, pBACsurf-1, plg, Signal plg, pYX, Selecta Vecta-Neo, Selecta Vecta-Hyg, and Selecta Vecta-Gpt from Novagen; pLexA, pB42AD, pGBT9, pAS2-1, pGAD424, pACT2, pGAD GL, pGAD GH, pGAD10, pGilda, pEZM3, pEGFP, pEGFP-1, pEGFP-N, pEGFP-C, pEBFP, pGFPuv, pGFP, p6xHis-GFP, pSEAP2-Basic, pSEAP2-Contral, pSEAP2-Promoter, pSEAP2-Enhancer, pβgal-Basic, pβgal-Control, pβgal-Promoter, pβgal-Enhancer, pCMV, pTet-Off, pTet-On, pTK-Hyg, pRetro-Off, pRetro-On, pIRES1 neo, pIRES1 hyg, pLXSN, pLNCX, pLAPSN, pMAMneo, pMAMneo-CAT, pMAMneo-LUC, pPUR, pSV2neo, pYEX4T-1/2/3, pYEX-S1, pBacPAK-His, pBacPAK8/9, pAcUW31, BacPAK6, pTriplEx, λgt10, λgt11, pWE15, and λTriplEx from Clontech; Lambda ZAP II, pBK-CMV, pBK-RSV, pBluescript II KS +/−, pBluescript II SK +/−, pAD-GAL4, pBD-GAL4 Cam, pSurfscript, Lambda FIX II, Lambda DASH, Lambda EMBL3, Lambda EMBL4, SuperCos, pCR-Scrigt Amp, pCR-Script Cam, pCR-Script Direct, pBS +/−, pBC KS +/−, pBC SK +/−, Phagescript, pCAL-n-EK, pCAL-n, pCAL-c, pCAL-kc, pET-3abcd, pET-11abcd, pSPUTK, pESP-1, pCMVLacI, pOPRSVI/MCS, pOPI3 CAT, pXT1, pSG5, pPbac, pMbac, pMC1neo, pMC1neo Poly A, pOG44, pOG45, pFRTβGAL, pNEOβGAL, pRS403, pRS404, pRS405, pRS406, pRS413, pRS414, pRS415, and pRS416 from Stratagene.

Additional vectors include, for example, pPC86, pDBLeu, pDBTrp, pPC97, p2.5, pGAD1-3, pGAD10, pACt, pACT2, pGADGL, pGADGH, pAS2-1, pGAD424, pGBT8, pGBT9, pGAD-GAL4, pLexA, pBD-GAL4, pHISi, pHISi-1, placZi, pB42AD, pDG202, pJK202, pJG4-5, pNLexA, pYESTrp and variants or derivatives thereof.

Selectable markers can also be inserted into the vector to allow for selection of cells that contain the NMDA receptor subunit. Suitable selectable marker include, but are not limited to: genes conferring the ability to grow on certain media substrates, such as the tk gene (thymidine kinase) or the hprt gene (hypoxanthine phosphoribosyltransferase) which confer the ability to grow on HAT medium (hypoxanthine, aminopterin and thymidine); the bacterial gpt gene (guanine/xanthine phosphoribosyltransferase) which allows growth on MAX medium (mycophenolic acid, adenine, and xanthine). See, for example, Song, K-Y., et al. Proc. Nat'l Acad. Sci. U.S.A. 84:6820-6824 (1987); Sambrook, J., et al., Molecular Cloning-A Laboratory Manual, Cold Spring Harbor Laboratory, Cold Spring Harbor, N.Y. (1989), Chapter 16. Other examples of selectable markers include: genes conferring resistance to compounds such as antibiotics, genes conferring the ability to grow on selected substrates, genes encoding proteins that produce detectable signals such as luminescence or fluorescence, such as green fluorescent protein, enhanced green fluorescent protein (eGFP). A wide variety of such markers are known and available, including, for example, antibiotic resistance genes such as the neomycin resistance gene (neo) (Southern, P., and P. Berg, J. Mol. Appl. Genet. 1:327-341 (1982)); and the hygromycin resistance gene (hyg) (Nucleic Acids Research 11:6895-6911 (1983), and Te Riele, H., et al., Nature 348:649-651 (1990)). Other selectable marker genes include: acetohydroxy acid synthase (AHAS), alkaline phosphatase (AP), beta galactosidase (LacZ), beta glucoronidase (GUS), chloramphenicol acetyltransferase (CAT), green fluorescent protein (GFP), red fluorescent protein (RFP), yellow fluorescent protein (YFP), cyan fluorescent protein (CFP), horseradish peroxidase (HRP), luciferase (Luc), nopaline synthase, octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracycline.

Methods for the incorporation of antibiotic resistance genes and negative selection factors will be familiar to those of ordinary skill in the art (see, e.g., WO 99/15650; U.S. Pat. No. 6,080,576; U.S. Pat. No. 6,136,566; Niwa, et al., J. Biochem. 113:343-349 (1993); and Yoshida, et al., Transgenic Research, 4:277-287 (1995)).

Additional selectable marker genes useful in this invention, for example, are described in U.S. Pat. Nos. 6,319,669; 6,316,181; 6,303,373; 6,291,177; 6,284,519; 6,284,496; 6,280,934; 6,274,354; 6,270,958; 6,268,201; 6,265,548; 6,261,760; 6,255,558; 6,255,071; 6,251,677; 6,251,602; 6,251,582; 6,251,384; 6,248,558; 6,248,550; 6,248,543; 6,232,107; 6,228,639; 6,225,082; 6,221,612; 6,218,185; 6,214,567; 6,214,563; 6,210,922; 6,210,910; 6,203,986; 6,197,928; 6,180,343; 6,172,188; 6,153,409; 6,150,176; 6,146,826; 6,140,132; 6,136,539; 6,136,538; 6,133,429; 6,130,313; 6,124,128; 6,110,711; 6,096,865; 6,096,717; 6,093,808; 6,090,919; 6,083,690; 6,077,707; 6,066,476; 6,060,247; 6,054,321; 6,037,133; 6,027,881; 6,025,192; 6,020,192; 6,013,447; 6,001,557; 5,994,077; 5,994,071; 5,993,778; 5,989,808; 5,985,577; 5,968,773; 5,968,738; 5,958,713; 5,952,236; 5,948,889; 5,948,681; 5,942,387; 5,932,435; 5,922,576; 5,919,445; and 5,914,233.

Cells that have successfully transformed to express a glutamate receptor can be confirmed via function analysis or molecular analysis. In one embodiment, cells, such as oocytes, in which NMDA receptor subunit cRNA has been inserted can be tested via electrophysiological recordings for the presence of functional NMDA receptors. In another embodiment, cells, in which the DNA encoding the NMDA receptor subunit gene(s) and a selectable marker gene has been inserted, can then be grown in appropriately-selected medium to identify cells providing the appropriate integration. Those cells which show the desired phenotype can then be further analyzed by restriction analysis, electrophoresis, Southern analysis, polymerase chain reaction, or another technique known in the art. By identifying fragments which show the appropriate insertion at the target gene site, cells can be identified in which homologous recombination has occurred to inactivate or otherwise modify the target gene.

Potency Boost Experiments

In a further aspect of the present invention, the potency boost of a compound can be determined, such as the compounds described according to the methods and processes herein.

In one aspect of the present invention, a process is provided to identify a compound that is useful to treat ischemic injury in a mammal, particularly a human, by: (i) assessing the potency boost of the compound at physiological pH versus “disorder-induced low pH” (for example, IC₅₀ at physiological pH/IC₅₀ at “disorder induced low pH”) in a cell by repeating the potency boost experiment at least 5 times such that the 95% confidence interval does not change more than 15% with the addition of a new experiment. In one embodiment, the potency boost can be determined is a cell that expressed a glutamate receptor. In a particular embodiment, the potency boost can be determined in a cell that expresses an NMDA receptor. In another embodiment, the cell can express an NR1 subunit and at least one NR2 subunit of an NMDA receptor. In a further embodiment, the NR2 subunit can be the NR2B subunit. In another embodiment, the NR2 subunit can be the NR2A subunit.

In another more general aspect of the present invention, a process is provided wherein a compound to treat a disorder that lowers the pH in a manner that activates an NMDA receptor antagonist is selected that (i) exhibits a potency boost of at least 5 as determined in experiments in which the potency boost of the compound is assessed at physiological pH versus “disorder-induced low pH” (for example, IC₅₀ at physiological pH/IC₅₀ at “disorder induced low pH”) as tested in a cell by repeating the potency boost experiments at least 5 times such that the 95% confidence interval does not change more than 15% with the addition of a new experiment. In one embodiment, the potency boost can be determined in a cell that expressed a glutamate receptor. In a particular embodiment, the potency boost can be determined in a cell that expresses an NMDA receptor. In another embodiment, the cell can express an NR1 subunit and at least one NR2 subunit of an NMDA receptor. In a further embodiment, the NR2 subunit can be the NR2B subunit. In another embodiment, the NR2 subunit can be the NR2A subunit.

In one embodiment, the potency boost of the compound can be determined by testing the effects of the compound at physiological pH versus “disorder-induced low pH” (for example, IC50 at physiological pH/IC50 at “disorder induced low pH”) in a cell expressing an NR1 subunit and at least one NR2 subunit of an NMDA receptor by repeating the potency boost experiment until the 95% confidence interval does not change more than 15% with the addition of a new experiment. In one particular embodiment, a method is provided to select a compound or a compound is selected that exhibits a potency boost of at least 5 as determined in experiments in which the potency boost of the compound is assessed at physiological pH versus “disorder-induced low pH” (for example, IC₅₀ at phys pH/IC₅₀ at “disorder induced low pH”) as tested in a cell expressing an NR1 subunit and at least one NR2 subunit of an NMDA receptor by repeating the potency boost experiments at least five times and until the 95% confidence interval does not change more than 15% with the addition of a new experiment. In one embodiment, the potency boost experiment at least 5 times such that the 95% confidence interval does not change more than 15% with the addition of a new experiment. In a further embodiment, the NR2 subunit can be the NR2B subunit. In another embodiment, the NR2 subunit can be the NR2A subunit.

In additional embodiments of the present invention, the NMDA receptor can contain any combination of the NR1 subunit in combination with at least one NR2 subunit, including NR2A, NR2B, NR2C and/or NR2D. For example, the NMDA receptor can contain any of the following subunits, including, but not limited to: NR1/NR2A, NR1/NR2B, NR1/NR2C, NR1/NR2D, NR1/NR2A/NR2B, NR1/NR2A/NR2C, NR1/NR2A/NR2D, NR1/NR2B/NR2C, NR1/NR2B/NR2D, NR1/NR2C/NR2D, NR1/NR2A/NR3A, NR1/NR2B/NR3A, NR1/NR2C/NR3A, NR1/NR2D/NR3A, NR1/NR2A/NR3B, NR1/NR2B/NR3B, NR1/NR2C/NR3B, NR1/NR2D/NR3B. In an alternative embodiment, the NMDA receptor of the present invention can contain an NR1 subuit and at least one NR3 subunit. For example, the NMDA receptor can contain any of the following, including, but not limited to: NR1/NR3A, NR1/NR3B, and/or NR1/NR3A/NR3B.

“Disorder-induced low pH” is defined as a drop in pH associated with any of the disorders or diseases referred to herein. The “disorder-induced low pH” can be between about 6.4 and about 7.2, generally about 6.9, 6.5, 6.6, 6.7, 6.8, 6.9, 7.0, or 7.1. Physiological brain-tissue pH is between about 7.2 and about 7.6, generally about 7.4, 7.3, or 7.5. In one embodiment, the “disorder-induced low pH” can be associated with an ischemic disorder, such as stroke.

“Potency boost” experiments determine the ratio of the concentrations of a compound that cause a half-maximal activation, potentiation, or inhibition of its receptor or target (EC₅₀ or IC₅₀ values) at physiological pH, such as pH 7.6, and ischemic pH, such as pH 6.9. Any method known in the art to determine EC₅₀ or IC₅₀ values for a compound can be used. The IC₅₀ values can be expressed as a ratio and averaged together to determine the mean shift in IC₅₀.

In one embodiment, two electrode voltage-clamp recordings can be used to determine IC₅₀ values for a compound. Glass microelectrodes can be filled with potassium chloride, such that the voltage electrode contains a lower concentration of potassium chloride than the current electrode. The cells can be placed in a chamber and perfused with physiological solution. External pH can be adjusted to either ischemic pH, such as pH 6.9 or physiological pH, such as pH 7.6. Dose response curves can then be obtained by applying in successive fashion maximally effective concentrations of glutamate and glycine, followed by glutamate/glycine plus variable concentrations of test compound. The level of inhibition by applied antagonist can be expressed as a percent of the initial glutamate response. These values can be averaged together across cells, for example across oocytes from a single frog. The average percent responses at each of the antagonist concentrations can be fitted by the logistic equation, (100−min)/(1+([conc]/IC50)^{nH})+min, where min is the residual percent response in saturating antagonist, IC₅₀ is the concentration of antagonist that causes half of the achievable inhibition, and nH is a slope factor describing steepness of the inhibitory curve. Min can be constrained to be greater than or equal to 0. For example, for experiments with known channel blockers, min can be set to 0. The IC₅₀ values obtained at physiological pH and ischemic pH can then be expressed as a ratio and averaged together to determine the mean shift in IC₅₀. In further embodiments, the compound can exhibit a potency boost of at least 6, 7, 8, 9, 10, 11, 12, 13, 14, 15, 16, 17, 18, 19, 20, 21, 22 or greater than 23 at physiological pH versus “disorder-induced low pH” (i.e., (IC₅₀ at phys pH)/(IC₅₀ at “disorder induced low pH”)).

The potency boost experiments can be repeated until the 95% confidence interval does not change more than 15% with the addition of a new experiment. In another embodiment, the potency boost experiments can be repeated until the 95% confidence interval does not change more than about 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3% or 2% with the addition of a new experiment. In a further embodiment, the potency boost experiments can be repeated until the 96%, 97%, 98% or 99% confidence interval does not change more than about 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3% or 2% with the addition of a new experiment.

II. In Vivo Assays

In Vivo Models of Transient Focal Ischemia

In one aspect of the present invention, a process is provided to identify a compound that is useful to treat ischemic injury in a mammal, particularly a human, by: (i) assessing the potency boost of the compound at physiological pH versus “disorder-induced low pH” (for example, IC₅₀ at physiological pH/IC₅₀ at “disorder induced low pH”) in a cell by repeating the potency boost experiment at least 5 times such that the 95% confidence interval does not change more than 15% with the addition of a new experiment; (ii) testing the compound in an animal model of transient focal ischemia and measuring the effect of the compound on the infarct volume by repeating the experiment at least 12 times such that the 95% confidence interval does not change more than 5% with the addition of a new experiment; (iii) selecting a compound that has a potency boost of at least 5 according to step (i) and at least a 30% decrease in infarct volume according to step (ii). According to the invention, a candidate drug must meet or exceed both the in vitro and in vivo criteria to be an effective drug for human use. In one embodiment, the potency boost can be determined in a cell that expresses a glutamate receptor. In another embodiment, the potency boost can be determined in a cell that expresses an NMDA, AMPA and/or kainate receptor. In one embodiment, the cell can express an NR1 subunit and at least one NR2 subunit of an NMDA receptor. In a further embodiment, the NR2 subunit can be the NR2B subunit. In another embodiment, the NR2 subunit can be the NR2A subunit.

In another more general aspect of the present invention, a process is provided wherein a compound is selected to treat a disorder that lowers the pH in a manner that activates an NMDA receptor antagonist that (i) exhibits a potency boost of at least 5 as determined in experiments in which the potency boost of the compound is assessed at physiological pH versus “disorder-induced low pH” (for example, IC₅₀ at physiological pH/IC₅₀ at “disorder induced low pH”) as tested in a cell by repeating the potency boost experiments at least 5 times such that the 95% confidence interval does not change more than 15% with the addition of a new experiment and (ii) exhibits at least a 30% decrease in infarct volume as measured in an animal model of focal ischemia as determined by repeating the experiment at least 12 times such that the 95% confidence interval does not change more than 5% with the addition of a new experiment. In one embodiment, the potency boost can be determined in a cell that expressed a glutamate receptor. In another embodiment, the potency boost can be determined in a cell that expresses an NMDA, AMPA and/or kainate receptor. In one embodiment, the cell can express an NR1 subunit and at least one NR2 subunit of an NMDA receptor. In a further embodiment, the NR2 subunit can be the NR2B subunit. In another embodiment, the NR2 subunit can be the NR2A subunit.

In a particular embodiment, a method is provided to select a compound or a compound is selected that exhibits at least a 30% decrease in infarct volume as measured in an animal model of focal ischemia as determined by repeating the experiment at least 15 times and until the 95% confidence interval does not change more than 10% with the addition of a new experiment. In another particular embodiment, the “disorder-induced low pH” can be associated with an ischemic disorder, such as stroke. In another embodiment, the middle cerebral artery occlusion model can be used as the animal model of transient focal ischemia, for example, in rodents, such as mice.

Focal ischemic stroke can be damage to the brain caused by interruption of the blood supply to a region thereof. The focal ischemic stroke is generally caused by obstruction of any one or more of the “main cerebral arteries” (e.g. middle cerebral artery, anterior cerebral artery, posterior cerebral artery, internal carotid artery, vertebral artery or basilar artery), as opposed to secondary arteries or arterioles. The arterial obstruction can be a single embolus or thrombus. Hence, focal ischemic stroke as defined herein is distinguished from the cerebral embolism stroke model (such as described in Bowes et al., Neurology 45:815-819 (1995)) in which a plurality of clot particles occlude secondary arteries or arterioles.

Focal ischemia can be induced in any mammal, including, but not limited to, rodents, mice, rats, rabbits and gerbils (see also Renolleau S, Stroke. 1998 July; 29(7):1454-60; Gotti, B. et al., Brain Res, 1990, 522, 290-307). For example, the gerbil has been widely used as an experimental model for studies of ischemic stroke because the brain blood supply is controlled by only two common carotid arteries. This unusual feature occurs in gerbils because they have an incomplete circle of Willis (Chandler et al., J. Pharmacol. Methods 14:137-146, 1985; Finkelstein et al., Restor. Neurol. Neurosci. 1:387-394, 1990; Levine and Sohn, Arch. Pathol. 87:315-317, 1969; Kahn, Neurology 22:510-515, 1972).

Test compounds can be administered to the animal prior to or after the occlusion of the artery. In one embodiment, the test compound can be administered intraperitoneally. In one embodiment, the test compound can be administered intracerebroventricularly. The test compound can be administered prior to the occlusion of the artery, for example, about 10, 20, 30, 40, 50 or 60 minutes prior to the ischemic event. Alternatively, test compound can be administered after the occlusion of the artery, for example, about 10, 20, 30, 40, 50, 60, 90, or 120 minutes or about 4, 6, 8 or 10 hours or about 1, 2, 3, 4, 5, 6, 7 or 8 days after the ischemic event, i.e. post-reperfusion.

The demonstration that compounds can protect cells in an ischemic area can be tested in animal models in which the middle cerebral artery (MCA) is experimentally occluded, namely the middle cerebral artery occlusion (MCAO) model. This animal model is well known in the art to simulate an in vivo ischemic event such as may occur in a human subject. The experimental occlusion of the MCA causes a large unilateral ischemic area that typically involves the basal ganglion and frontal, parietal, and temporal cortical areas (Menzies et al. Neurosurgery 31, 100-106 (1992)). The ischemic lesion begins with a smaller core at the site perfused by the MCA and grows with time. This penumbral area around the core infarct is believed to result from a propagation of the lesion from the core outward to tissue that remains perfused by collateral circulation during the occlusion. The effect of a therapeutic agent on the penumbra surrounding the core of the ischemic event may be examined when brain slices are obtained from the animal. The MCA supplies blood to the cortical surfaces of frontal, parietal, and temporal lobes as well as basal ganglia and internal capsule. Slices of the brain can be taken around the region where the greatest ischemic effect occurs. The MCAO can be induced in any mammal, including, but not limited to, mice, rats, rabbits and gerbils, (see also Renolleau S, Stroke. 1998 July; 29(7):1454-60; Gotti, B. et al., Brain Res, 1990, 522, 290-307). The MCA model allows for an indirect measure of neuronal cell death following an ischemic event (i.e., occlusion of the left middle cerebral artery). In one embodiment, a transient focal cerebral ischemia of the middle cerebral artery can be used to test the compounds.

Transient focal cerebral ischemia can be induced by intraluminal middle cerebral artery (MCA) occlusion. Occlusion can be achieved through any means that blocks the artery, for example, with a suture, such as a monofilament suture. After the animals are anesthetized, a probe can be affixed to their skull to monitor relative changes in regional cerebral blood flow. Such changes can be monitored with a laser Doppler flowmeter (Perimed). For example, in mice, the probe can be affixed 2 mm posterior and 4-6 mm lateral of the bregma. Then, an incision can be made to access the MCA and a material can be inserted to occlude the MCA. For example, a suture can be introduced into the internal carotid artery through the external carotid artery stump until monitored blood flow is stopped. After a period of time of MCA occlusion, such as about 30 minutes, 45 minutes or 60 minutes, blood flow can be restored by withdrawing the blocking material.

In another embodiment, a bilateral carotid occlusion model can be used to demonstrate that compounds can protect cells in an ischemic area. Animals can be anesthetized and an incision can be made in the ventral neck and the common carotid arteries can be isolated and occluded completely for a period of time, for example 5, 10, 15, 20, 30, 45 or 60 minutes. The artery can be occluded by any means, for example, using a clip, such as a microaneurysm clips. The occlusion can then be stopped and the incision can be sutured. In one particular embodiment, the bilateral carotid occlusion can be conducted in a gerbil.

After surgery, the animals can then be allowed to recover. After the animal survives for a period of time, for example, about 12, 24, 36, 48 or 72 hours, the animal can be sacrificed and the brain removed and sectioned, for example in approximately, 1, 2, 3, 4, 5 or 10 mm sections. The volume of infarct can then be identified by staining the brain sections with an appropriate dye, for example 2% 2,3,5-triphenyltetrazolium chloride (TTC) in PBS at 37° C. for approximately 20 minutes. The infarct area of each section can then be measured and multiplied by the section thickness to give the infarct volume of that section. A ratio of the contralateral to ipsilateral hemisphere section volume can also be multiplied by the corresponding infarct section volume to correct for edema. Infarct volume can be determined by summing the infarct area times section thickness for all sections.

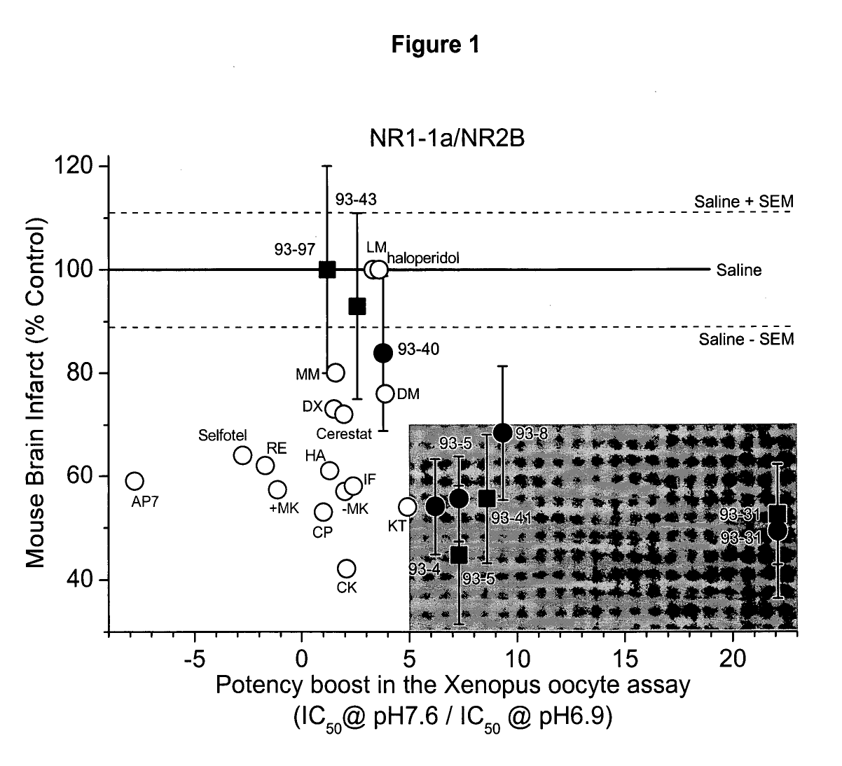

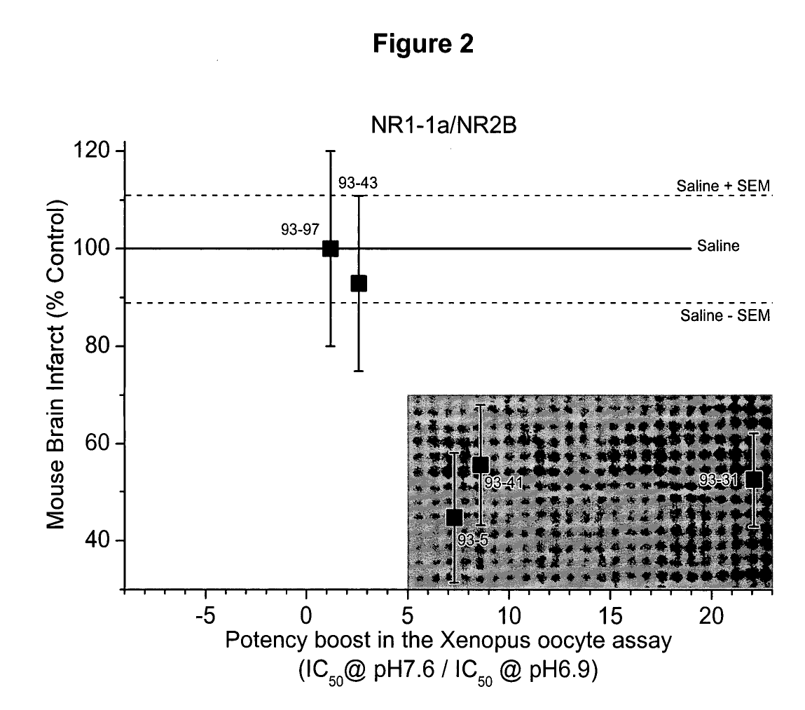

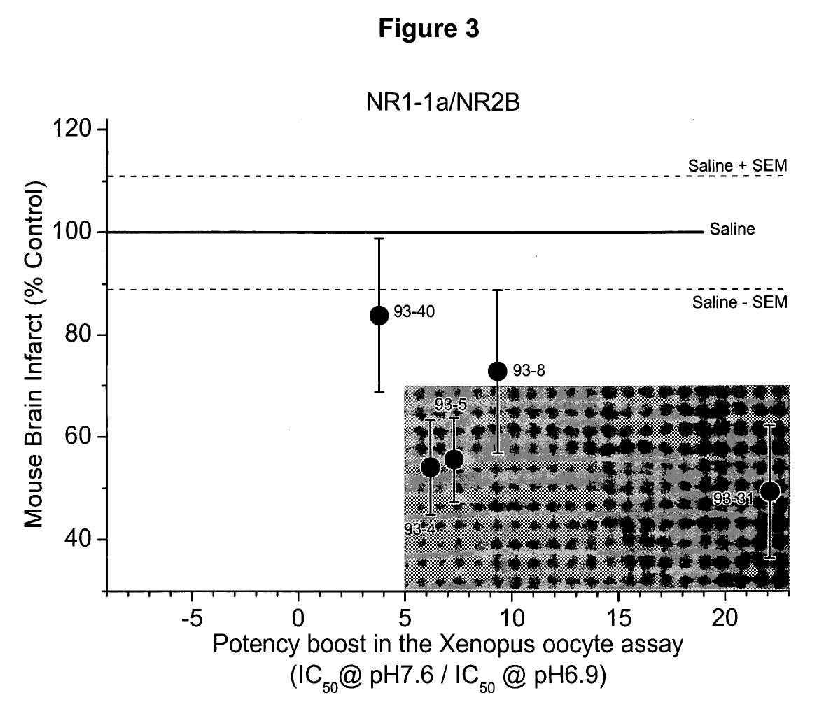

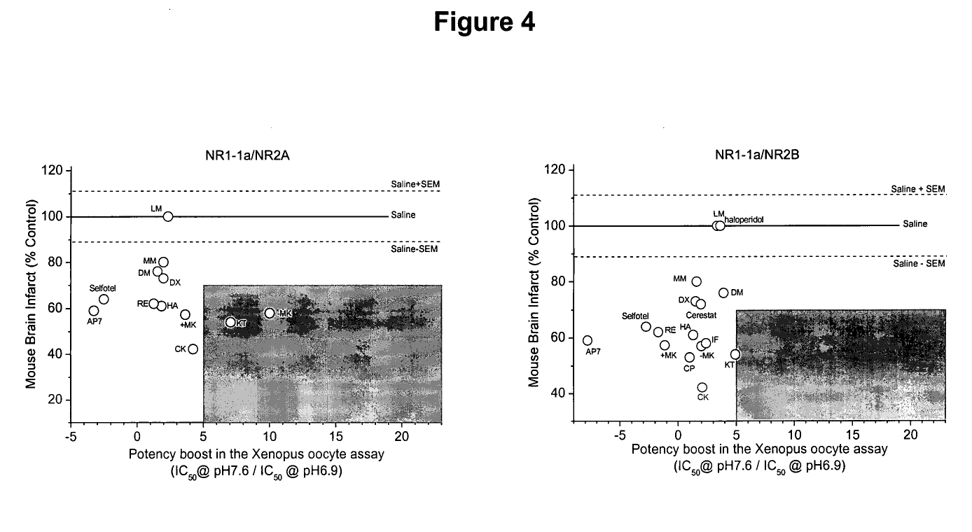

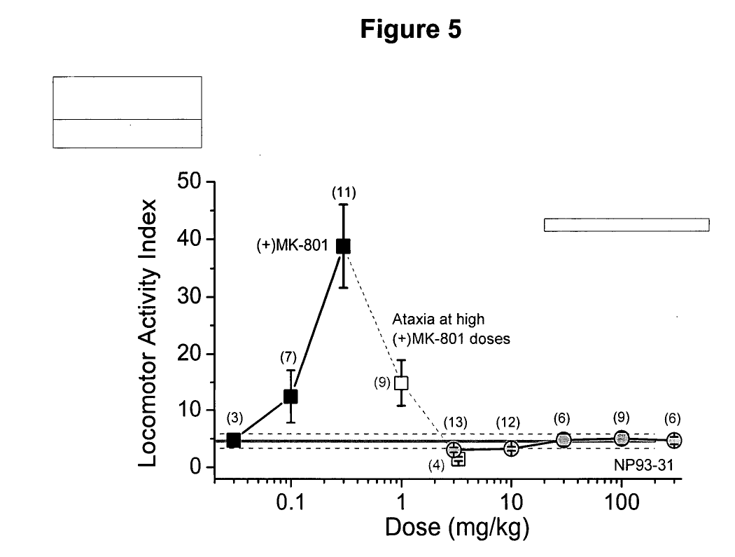

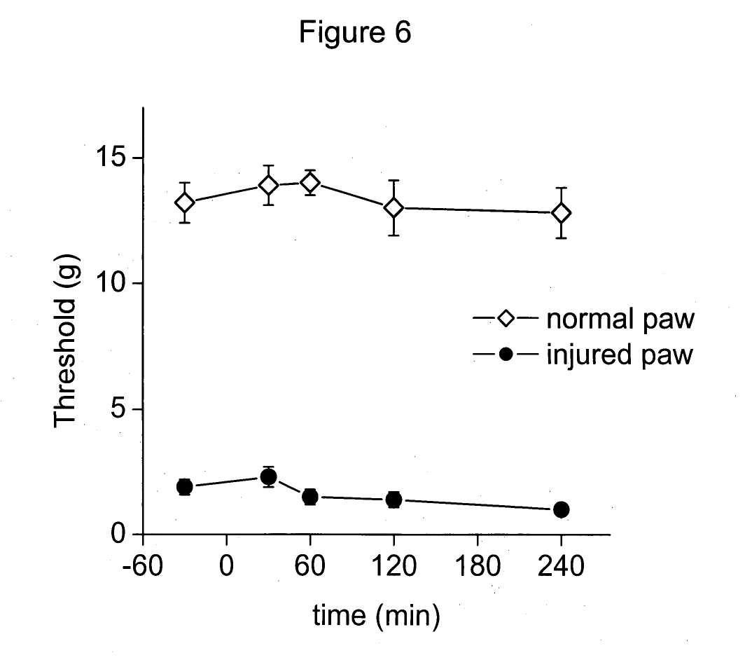

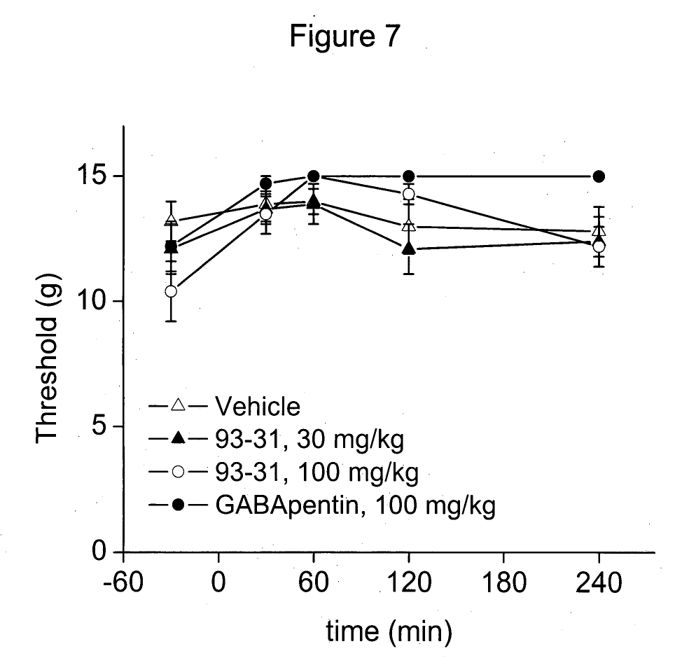

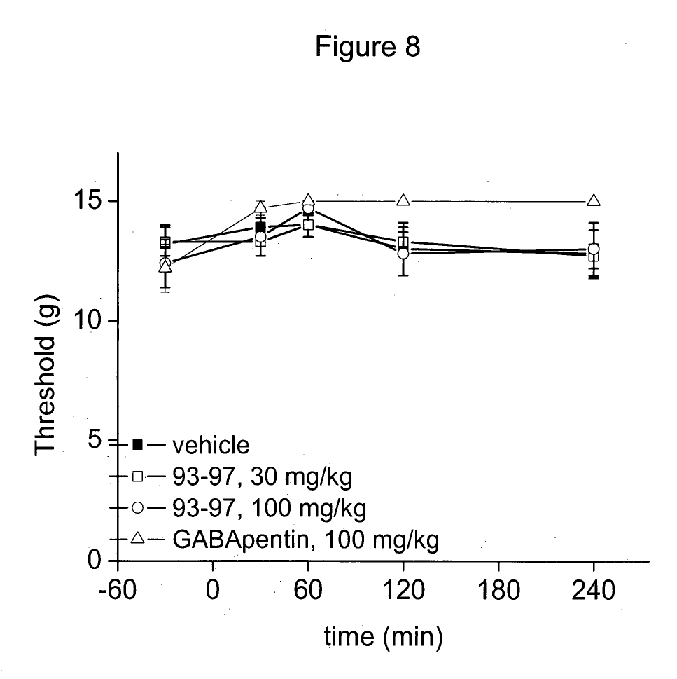

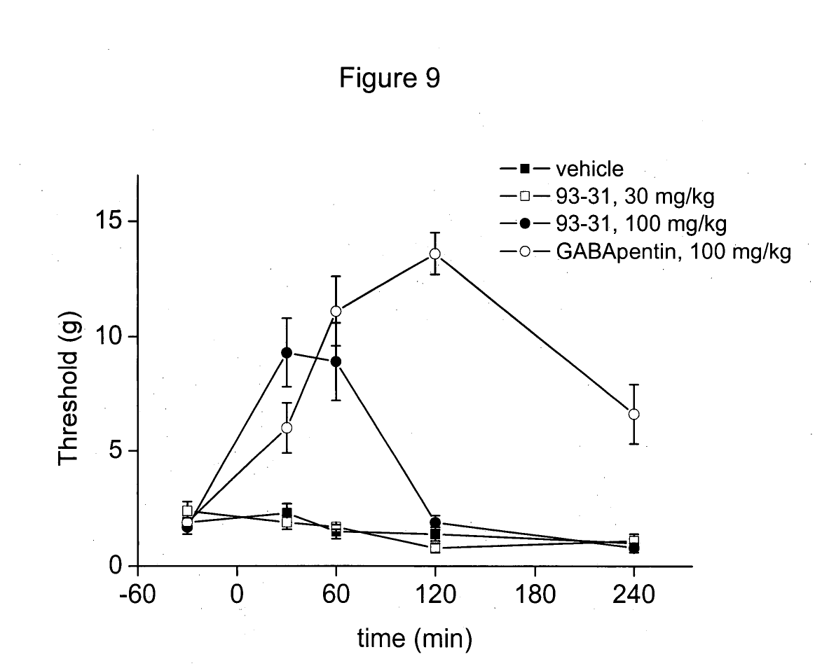

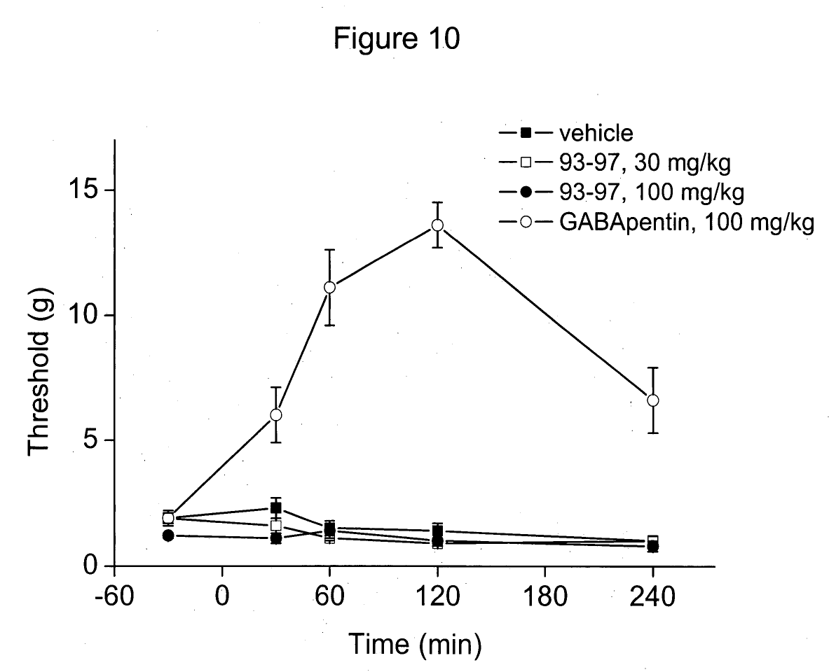

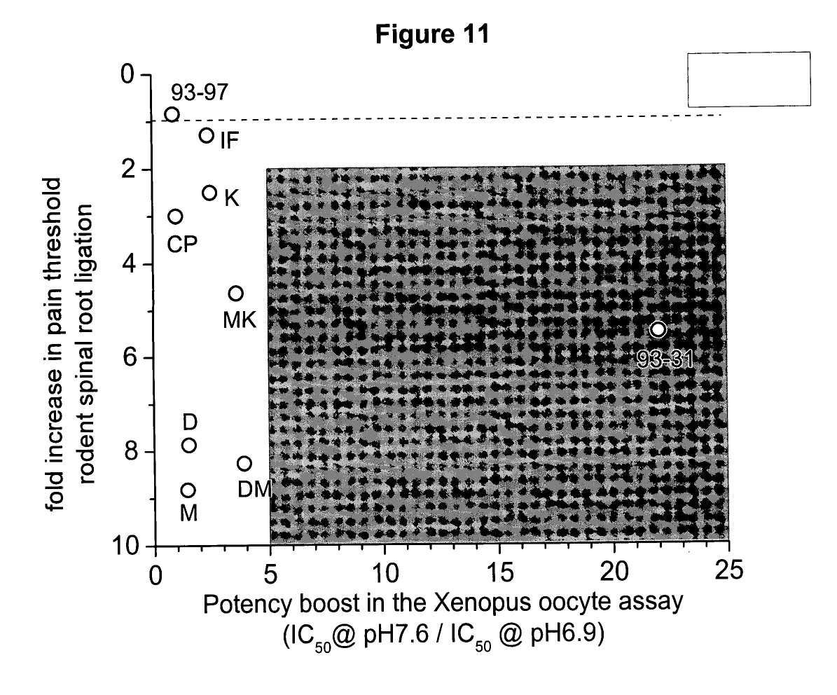

After testing the compound in an animal model of transient focal ischemia and measuring the effect of the compound on the infarct volume, compounds can be selected that result in at least a 30% decrease in infarct volume. In additional embodiments, compounds can be selected that result in at least a 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 97, 99 or 100% decrease in infarct volume. In further embodiments, the compound can exhibit a potency boost of at least 5, 6, 7, 8, 9, 10, 11, 12, 13, 14, 15, 16, 17, 18, 19, 20, 21, 22, 23, 24, 25, 30, 40 or 50 at physiological pH versus ischemic pH (i.e., phys pH/Isc pH) and at least a 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 97, 99 or 100% decrease in infarct volume, such as illustrated in FIG. 1, including independently, any combination of these numbers, each combination of which is deemed to be specifically disclosed. In certain embodiments of the present invention the mean, i.e. the sum of all the observations divided by the number of observations, can be calculated for the potency boost and infarct volume experiments and the mean value of the compound can exhibit a potency boost of at least 5, 6, 7, 8, 9, 10, 11, 12, 13, 14, 15, 16, 17, 18, 19, 20, 21, 22 or 23 at physiological pH versus ischemic pH (i.e., phys pH/Isc pH) and at least a 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80 or 80% decrease in infarct volume, such as illustrated in FIG. 1.

The infarct volume experiments can be repeated until the 95% confidence interval does not change more than 10% with the addition of a new experiment. In another embodiment, the infarct volume experiments can be repeated until the 95% confidence interval does not change more than about 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3% or 2% with the addition of a new experiment. In a further embodiment, the infarct volume experiments can be repeated until the 96%, 97%, 98% or 99% confidence interval does not change more than about 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3% or 2% with the addition of a new experiment.

Other animal models of transient focal ischemia include, but are not limited to intra-arterial injection of microspheres or coagulated blood, four vessel occlusion in rat, two vessel occlusion in gerbil, or photochemicaly induced clot formation with dissolution. Such models are known to one skilled in the art.

Animal Models of Neuropathic Pain

In one aspect of the present invention, the compounds disclosed herein can be used for the treatment of neuropathic pain and related disorders.

In one aspect of the present invention, a process is provided to identify a chemical compound that is useful to treat neuropathic pain in a mammal, particularly a human, by: (i) assessing the potency boost of the compound at physiological pH versus “disorder-induced low pH” (for example, IC50 at phys pH/IC50 at “disorder induced low pH”) in a cell by repeating the potency boost experiment at least 5 times such that the 95% confidence interval does not change more than 15% with the addition of a new experiment; (ii) testing the compound in an animal model of neuropathic pain and measuring the effect of the compound on the increase in pain threshold by repeating the experiment at least 12 times such that the 95% confidence interval does not change more than 5% with the addition of a new experiment; (iii) selecting a compound that has a potency boost of at least 5 according to step (i) and at least a 2-fold increase in pain threshold according to step (ii). According to the invention, a candidate drug must meet or exceed both the in vitro and in vivo criteria to be a effective drug for human use. In one embodiment, the potency boost can be determined is a cell that expressed a glutamate receptor. In another embodiment, the potency boost can be determined in a cell that expresses an NMDA, AMPA, and/or kainate receptor. In one embodiment, the cell can express an NR1 subunit and at least one NR2 subunit of an NMDA receptor. In a further embodiment, the NR2 subunit can be the NR2B subunit. In another embodiment, the NR2 subunit can be the NR2A subunit.

In another more general aspect of the present invention, a process is provided wherein a compound to treat a disorder that lowers the pH in a manner that activates an NMDA receptor antagonist is selected that (i) exhibits a potency boost of at least 5 as determined in experiments in which the potency boost of the compound at assessing the potency boost of the compound at physiological pH versus “disorder-induced low pH” (for example, IC50 at phys pH/IC50 at “disorder induced low pH”) is tested in a cell by repeating the potency boost experiments at least 5 times such that the 95% confidence interval does not change more than 15% with the addition of a new experiment and (ii) testing the compound in an animal model of neuropathic pain and measuring the effect of the compound on the increase in pain threshold by repeating the experiment at least 12 times such that the 95% confidence interval does not change more than 5% with the addition of a new experiment; (iii) selecting a compound that has a potency boost of at least 5 according to step (i) and at least a 2-fold increase in pain threshold according to step (ii). In one embodiment, the potency boost can be determined is a cell that expressed a glutamate receptor. In another embodiment, the potency boost can be determined in a cell that expresses an NMDA, AMPA, and/or kainate receptor. In one embodiment, the cell can express an NR1 subunit and at least one NR2 subunit of an NMDA receptor. In a further embodiment, the NR2 subunit can be the NR2B subunit. In another embodiment, the NR2 subunit can be the NR2A subunit.

In one embodiment, the animal model of neuropathic pain can be selected from the group including, but not limited to: the chronic constriction injury model, the partial sciatic ligation model, the spinal nerve ligation model or any other model known to one skilled in the art. In a particular embodiment, the spinal nerve ligation model can be used as the in vivo animal model.

The pain threshold can be defined the amount of stimulation required before the sensation of pain is experienced. In neuropathic pain models, animals are subject to injury such that a state of chronic pain is induced. Noxious stimuli can then be applied and the amount of time that the animal can tolerate the noxious stimuli without reacting to it can be calculated. For example, an uninjured animal could be exposed to a cold surface for 20 minutes before withdrawing its paw from the surface, but after an injury, such as one described below to model neuropathic pain, the animal may withdraw its paw after only 1 minute. Examples of noxious stimuli include, but are not limited to: heat, cold, mechanical, such as von Frey's stimulus, chemical and the like.

In one embodiment, the chronic constriction injury model (CCI, or the Bennett model) can be used as the animal model of neuropathic pain (see, for example, Bennett, Gary J. et al. Pain, 1988, 33, 87-107). In this model, the sciatic nerve of an animal, foe example, a rat, can be intentionally injured in a manner that was discovered to induce symptoms reported by human patients with neuropathic pain. Specifically, the sciatic nerve can be exposed at midthigh, proximal to the nerve's trifurcation in the popliteal fossa. At that location, about 7 mm of the nerve's trajectory can be freed of adhering tissue and four ligatures tied loosely around it, with about 1-mm spacing. In each animal, an identical dissection can be performed contralaterally without ligation so that each animal can serve as its own control. On the ligated side, the affected hindpaw skin becomes unequivocally hyperalgesic and allodynic (i.e., experiences pain resulting from a stimulus that ordinarily does not elicit a painful response), and perhaps a source of spontaneous pain as well. To test for hyperalgesia, a noxious stimuli, such as heat, can be aimed at the plantar hindpaw from beneath a glass floor and the latency for paw withdrawal (a marker for pain threshold) can be measured. The responses on the nerve-injured side tend to be of abnormal magnitude and duration, exceeding, for example, 30 seconds of paw elevation, and can be accompanied by prolonged licking. A normal response would be that the animal barely raise the paw and would last less than a second or two. To test for cold allodynia, the animals can be placed on a metal floor cooled, for example, at a temperature of 4° C. To an unligated paw, the floor produces no pain, even after 20 minutes of contact. Rats with ligation can be measured for withdrawals of the nerve-injured paw, which, for example, can increase more than fivefold, and the duration can be measured, it can increase, for example, more than twofold. Using such a model, pain threshold can be calculated without drug and also after administration of a compound described herein.

In another embodiment, the partial sciatic ligation model (the Seltzer model) can be used to test neuropathic pain threshold (see, Seltzer, A. et al. Pain, 1990, 43, 205-218). In this model, half of the sciatic nerve high in the thigh of an animal, such as a rat, can be unilaterally ligated. Within a few hours after the operation, and for several months thereafter, the animals can develop guarding behavior of the ipsilateral hind paw and lick it often, suggesting the possibility of spontaneous pain. The plantar surface of the foot can be evenly hyperesthetic to non-noxious and noxious stimuli. Common measurements to noxious stimuli can be measured in the animal with and without exposure to the compounds of the present invention. Noxious stimuli can include the Von Frey hair stimulation, CO₂ laser heat pulses and pin procks. In response to repetitive Von Frey hair stimulation at the plantar side, there can be a sharp decrease in the withdrawal thresholds. After a series of such stimuli in the operated side, light touch elicits aversive responses, suggesting allodynia to touch. The withdrawal thresholds to CO₂ laser heat pulses is also markedly lowered. Suprathreshold noxious heat pulses elicit exaggerated responses unilaterally, suggesting thermal hyperalgesia. Pin-pricks also can evoke such exaggerated responses (mechanical hyperalgesia). Using such a model, pain threshold can be calculated without drug and also after administration of a compound described herein.

In another embodiment, the spinal nerve ligation model (the Chung model) can be used to measure neuropathic pain (see Kim, S. H. and Chung, J. M. Neurosci. Lett. 1991, 134, 131-134; Kim, S. H. and Chung, J. M. Pain, 1992, 50, 355-363). In this model, the L₅ (or L₅+L₆) spinal nerves are tightly ligated and then cut. The surgical procedure produces a long-lasting hyperalgesia to noxious heat and mechanical allodynia of the affected foot. Mechanical sensitivity of the affected hind paw can be measured. It can be significantly elevated from the first day after the surgery as evidenced by the increased occurrence of foot withdrawal to innocuous mechanical stimulation applied with von Frey filaments to the hind paw. In addition, behavioral signs of the presence of spontaneous pain in the affected foot are also seen. Such measurements can be determined with and without administration of a compound of the present invention and pain thresholds can be calculated.

After testing the compound in an animal model of neuropathic pain and measuring the effect of the compound on the pain threshold, compounds can be selected that result in at least a 2-fold increase in pain threshold. In other embodiments, the compound can exhibit at least a 3, 4, 5, 6, 7, 8, 9, 10, 15, 20 or 30 fold increase in pain threshold. In a further embodiment, the experiment can be repeated at least 15 times and until the 95% confidence interval does not change more than 10% with the addition of a new experiment. The neuropathic pain experiments can be repeated until the 95% confidence interval does not change more than 10% with the addition of a new experiment. In another embodiment, the neuropathic pain experiments can be repeated until the 95% confidence interval does not change more than about 9%, 8%, 7%, 6%, 5%, 4%, 3% or 2% with the addition of a new experiment. In a further embodiment, the neuropathic pain experiments can be repeated until the 96%, 97%, 98% or 99% confidence interval does not change more than about 10%, 9%, 8%, 7%, 6%, 5%, 4%, 3% or 2% with the addition of a new experiment.

Other animal models of neuropathic pain include, but are not limited to, the spared nerve injury model (see Decosterd & Woolf. Pain. 2000 August; 87(2):149-58), sciatic inflammatory neuropathy (SIN) induced by localized inflammation of the sciatic nerve in the absence of frank trauma, and/or a peripheral nerve model of pain following the injection of the chemotherapeutic agent vincristine (Aley et al Neurosci 1996; 73:259-65). Additional models are known to one skilled in the art. See also Zimmerman M. Eur J Pharmacol 2001; 429:23-37; Shir et al Neurosci Lett 1990; 115:62-7. Wall et al Pain 1979; 7:103-11; DeLeo et al Pain 1994; 56:9-16; Courteix et al Pain 1994; 57:153-60; Aley et al; Slart et al Pain 1997; 69:119-25; Hargreaves et al Pain 1988; 32:77-88.

III. Compounds

In one aspect of the present invention, a process is provided to identify a compound that is useful to treat ischemic injury in a mammal, particularly a human, by: (i) assessing the potency boost of the compound at physiological pH versus “disorder-induced low pH” (for example, IC₅₀ at physiological pH/IC₅₀ at “disorder induced low pH”) in a cell by repeating the potency boost experiment at least 5 times such that the 95% confidence interval does not change more than 15% with the addition of a new experiment; (ii) testing the compound in an animal model of transient focal ischemia and measuring the effect of the compound on the infarct volume by repeating the experiment at least 12 times such that the 95% confidence interval does not change more than 5% with the addition of a new experiment; (iii) selecting a compound that has a potency boost of at least 5 according to step (i) and at least a 30% decrease in infarct volume according to step (ii). According to the invention, a candidate drug must meet or exceed both the in vitro and in vivo criteria to be an effective drug for human use. In one embodiment, the potency boost can be determined in a cell that expresses a glutamate receptor. In another embodiment, the potency boost can be determined in a cell that expresses an NMDA, AMPA and/or kainate receptor. In one embodiment, the cell can express an NR1 subunit and at least one NR2 subunit of an NMDA receptor. In a further embodiment, the NR2 subunit can be the NR2B subunit. In another embodiment, the NR2 subunit can be the NR2A subunit.

In another more general aspect of the present invention, a process is provided wherein a compound is selected to treat a disorder that lowers the pH in a manner that activates an NMDA receptor antagonist that (i) exhibits a potency boost of at least 5 as determined in experiments in which the potency boost of the compound is assessed at physiological pH versus “disorder-induced low pH” (for example, IC₅₀ at physiological pH/IC₅₀ at “disorder induced low pH”) as tested in a cell by repeating the potency boost experiments at least 5 times such that the 95% confidence interval does not change more than 15% with the addition of a new experiment and (ii) exhibits at least a 30% decrease in infarct volume as measured in an animal model of focal ischemia as determined by repeating the experiment at least 12 times such that the 95% confidence interval does not change more than 5% with the addition of a new experiment. In one embodiment, the potency boost can be determined in a cell that expressed a glutamate receptor. In another embodiment, the potency boost can be determined in a cell that expresses an NMDA, AMPA and/or kainate receptor. In one embodiment, the cell can express an NR1 subunit and at least one NR2 subunit of an NMDA receptor. In a further embodiment, the NR2 subunit can be the NR2B subunit. In another embodiment, the NR2 subunit can be the NR2A subunit.

In one particular embodiment, a process is provided to identify a chemical compound that is useful to treat ischemic injury in a mammal, particularly a human, by: (i) assessing the potency boost of the compound at physiological pH versus “disorder-induced low pH” (for example, IC₅₀ at physiological pH/IC₅₀ at “disorder induced low pH”) in a cell expressing an NR1 subunit and at least one NR2 subunit of an NMDA receptor by repeating the potency boost experiment at least 5 times such that the 95% confidence interval does not change more than 15% with the addition of a new experiment; (ii) testing the compound in an animal model of transient focal ischemia and measuring the effect of the compound on the infarct volume by repeating the experiment at least 12 times such that the 95% confidence interval does not change more than 5% with the addition of a new experiment; (iii) selecting a compound that has a potency boost of at least 5 according to step (i) and at least a 30% decrease in infarct volume according to step (ii). According to the invention, a candidate drug must meet or exceed both the in vitro and in vivo criteria to be an effective drug for human use. In one embodiment, the NR2 subunit can be the NR2B subunit. In another embodiment, the NR2 subunit can be the NR2A subunit.

In another particular embodiment, a process is provided wherein a compound to treat a disorder that lowers the pH in a manner that activates an NMDA receptor antagonist is selected that (i) exhibits a potency boost of at least 5 as determined in experiments in which the potency boost of the compound is assessed at physiological pH versus “disorder-induced low pH” (for example, IC₅₀ at physiological pH/IC₅₀ at “disorder induced low pH”) is tested in a cell expressing an NR1 subunit and at least one NR2 subunit of an NMDA receptor by repeating the potency boost experiments at least 5 times such that the 95% confidence interval does not change more than 15% with the addition of a new experiment and (ii) exhibits at least a 30% decrease in infarct volume as measured in an animal model of focal ischemia as determined by repeating the experiment at least 12 times such that the 95% confidence interval does not change more than 5% with the addition of a new experiment. In one embodiment, the NR2 subunit can be the NR2B subunit. In another embodiment, the NR2 subunit can be the NR2A subunit.

Further, in additional embodiments, the compound does not exhibit toxicity, such as, for example, motor impairment, cognitive impairment and cardiac toxicity or those described herein. Additionally or alternatively, the compound can be at least 10 times more selective for binding to the NMDA receptor than any other glutamate receptor other receptor as described herein. In a further additional or alternative embodiment, the compound can have a therapeutic index equal to or greater than at least 2:1.

In another embodiment of the present invention, a process is provided to identify a chemical compound that is useful to treat ischemic injury in a human by: (i) assessing the potency boost of the compound at physiological pH versus “disorder-induced low pH” in a cell expressing a NR1/NR2A NMDA receptor and/or a NR1/NR2B NMDA receptor by repeating the potency boost experiment until the 95% confidence interval does not change more than 10% with the addition of a new experiment; (ii) testing the compound in an animal model of transient focal ischemia and measuring the effect of the compound on the infarct volume by repeating the experiment until the 95% confidence interval does not change more than 10% with the addition of a new experiment; (iii) selecting a compound that has a potency boost of at least 5 according to step (i) and at least a 30% decrease in infarct volume according to step (ii).

In a further embodiment of the present invention, a process is provided to select a compound to treat a disorder that is associated with ischemic injury that (i) exhibits a potency boost of at least 5 as determined in experiments in which the potency boost of the compound at physiological pH versus “disorder-induced low pH” is tested in a cell expressing a NR1/NR2A NMDA receptor and/or a NR1/NR2B NMDA receptor by repeating the potency boost experiments at leat 5 times or until the 95% confidence interval does not change more than 10% with the addition of a new experiment and (ii) exhibits at least a 30% decrease in infarct volume as measured in an animal model of focal ischemia as determined by repeating the experiment until the 95% confidence interval does not change more than 10% with the addition of a new experiment.

Additionally or alternatively, the compound can be at least 10 times more selective for binding to the NMDA receptor than any other glutamate receptor. In other embodiments, the compound can be at least 11, 12, 13, 14, 15, 16, 17, 18, 19, 20, 25, 30, 35, 40, 45, 50, 55, 60, 65, 70, 75, 78, 85, 90, 95, 100, 125, 150, 175, 200, 300, 400, 500, or 1000 times more selective for binding to the NMDA receptor than any other glutamate receptor, for example, including, but not limited to the following glutamate receptors: AMPA GluR1 (GenEMBL Accession Nos. X57497, X17184, I57354), AMPA GluR2 (GenEMBL Accession Nos. X57498, M85035, A46056), AMPA GluR3 (GenEMBL Accession Nos. M85036, X82068), AMPA GluR4 (GenEMBL Accession Nos. M36421, U16129), Kainate GluR5 (GenEMBL Accession Nos. X66118, M83560, U16125), Kainate GluR6 (GenEMBL Accession Nos. D10054, Z11715, U16126), Kainate GluR7 (GenEMBL Accession Nos. M83552, U16127), Kainate KA-1 (GenEMBL Accession Nos. X59996, S67803a), Kainate KA-2 (GenEMBL Accession Nos. D10011, Z11581, S40369), Orphan d1 GRID1 (GenEMBL Accession Nos. D10171, Z17238), Orphan d2 GRID2 (GenEMBL Accession Nos. D13266, Z17239), and/or metabotropic glutamate receptors (mGluR5), such as Group 1 mGluR5, including mGluR 1 and mGluR 5, Group 2 mGluR5, including, mGluR 2 and mGluR 3, and Group 3 mGluR5, including mGluR 4, mGluR 6, mGluR 7, and mGluR 8. The NMDA receptor can be made up of any of its subunits, including, but not limited to NMDA NR1 (Chromosome (human) 9q34.3, GenEMBL Accession No. for Mouse: D10028, GenEMBL Accession No. for Rat: X63255, GenEMBL Accession No. for Human: X58633), NMDA NR2A (Chromosome (human): 16p13.2, GenEMBL Accession No. for Mouse: D10217, GenEMBL Accession No. for Rat: D13211, GenEMBL Accession No. for Human: U09002); NMDA NR2B (Chromosome (human): 12p12 GenEMBL Accession No. for Mouse: D10651′ GenEMBL Accession No. for Rat: M91562, GenEMBL Accession No. for Human: U28861a); NMDA NR2C (Chromosome (human) 17q24-q25, GenEMBL Accession No. for Mouse: D10694, GenEMBL Accession No. for Rat: D13212); NMDA NR2D (Chromosome (human) 19q13.1qter, GenEMBL Accession No. for Mouse: D12822, GenEMBL Accession No. for Rat: D13214, GenEMBL Accession No. for Human: U77783); NMDA NR3A (GenEMBL Accession No. for Rat: L34938 and/or NMDA NR3B. Alternatively, the compound is not more selective or at least 2, 3, 4, 5, 6, 7, 8, or 9 times more selective for the NMDA receptor then another glutamate receptor listed above.

Additionally or alternatively, the compound can be at least 10 times more selective for binding to the NMDA receptor than another receptor type. In other embodiments, the compound can be at least 11, 12, 13, 14, 15, 16, 17, 18, 19, 20, 25, 30, 35, 40, 45, 50, 55, 60, 65, 70, 75, 78, 85, 90, 95, 100, 125, 150, 175, 200, 300, 400, 500, or 1000 times more selective for binding to the NMDA receptor than another receptor type, for example, including, but not limited to the following receptors: dopamine receptors, such as D1, D2, D3, D4 and D5 dopamine receptors; opioid receptors, such as mu opioid receptors, including mu1 and mu2; delta opioid receptors, including delta1 and delta2, and kappa opioid receptors, including kappa1 and kappa 2; cholinergic receptors, including muscarinic and nicotinic receptors; adrenergic receptors, including epinephrine receptors and epinephrine receptors, GABA receptors, including GABA-A and GABA-B receptors, or a peptide receptor, such as, but not limited to the receptors for the peptides listed in Table B below. Alternatively, the compound is not more selective or at least 2, 3, 4, 5, 6, 7, 8, or 9 times more selective for the NMDA receptor then a receptor listed above.

TABLE B

Hypothalamic hormones
Oxytocin
Vasopressin
Hypothalamic releasing and inhibiting hormones
Corticotropin releasing hormone (CRH)
Growth hormone releasing hormone (GHRH)
Luteinizing hormone releasing hormone (LHRH)
Somatostatin growth hormone release inhibiting hormone
Thyrotropin releasing hormone (TRH)
Tachykinins
Neurokinin a (substance K)
Neurokinin b
Neuropeptide K
Substance P
Opioid peptides
beta-endorphin
Dynorphin
Met- and leu-enkephalin
NPY and related peptides
Neuropeptide tyrosine (NPY)
Pancreatic polypeptide
Peptide tyrosine-tyrosine (PYY)
VIP-glucagon family
Glucogen-like peptide-1 (GLP-1)
Peptide histidine isoleucine (PHI)
Pituitary adenylate cyclase activating peptide (PACAP)
Vasoactive intestinal polypeptide (VIP)
Other peptides
Brain natriuretic peptide
Calcitonin gene-related peptide (CGRP) (a- and b-form)
Cholecystokinin (CCK)
Galanin
Islet amyloid polypeptide (IAPP) or amylin
Melanin concentrating hormone (MCH)
Melanocortins (ACTH, a-MSH)
Neuropeptide FF (F8Fa)
Neurotensin
Parathyroid hormone related protein
Agouti gene-related protein (AGRP)
Cocaine and amphetamine regulated transcript (CART)
peptide
Endomorphin-1 and -2
5-HT-moduline
Hypocretins/orexins
Nociceptin/orphanin FQ
Nocistatin
Prolactin releasing peptide
Secretoneurin
Urocortin

In another embodiment, the compound can be at least 10, 11, 12, 13, 14, 15, 16, 17, 18, 19, 20, 25, 30, 35, 40, 45, 50, 55, 60, 65, 70, 75, 78, 85, 90, 95, 100, 125, 150, 175, 200, 300, 400, 500, or 1000 times more selective for binding to the NMDA receptor than a serotonin receptor. Alternatively, the compound is not more selective or at least 2, 3, 4, 5, 6, 7, 8, or 9 times more selective for the NMDA receptor then a serotonin receptor. Seratonin receptors include, but are not limited to 5HT₁, including 5HT_{1A}, 5HT_{1B}, 5HT_{1D}, 5HT_{1E}, and 5HT_{1F}; 5HT₂, including 5HT_{2A}, 5HT_{2B}, and 5HT_{2C}; 5HT₃; 5HT₄; 5HT₅, including 5HT₅ₐ and 5HT_{5B}; 5HT₆ and 5HT₇. In another embodiment, the compound can be at least 10, 11, 12, 13, 14, 15, 16, 17, 18, 19, 20, 25, 30, 35, 40, 45, 50, 55, 60, 65, 70, 75, 78, 85, 90, 95, 100, 125, 150, 175, 200, 300, 400, 500, or 1000 times more selective for binding to the NMDA receptor than a histamine receptor, including H1, H2, H3 and H4 histamine receptors. Alternatively, the compound is not more selective or at least 2, 3, 4, 5, 6, 7, 8, or 9 times more selective for the NMDA receptor then a histamine receptor, including H1, H2, H3 and H4 histamine receptors. In another embodiment, the compound can be at least 10, 11, 12, 13, 14, 15, 16, 17, 18, 19, 20, 25, 30, 35, 40, 45, 50, 55, 60, 65, 70, 75, 78, 85, 90, 95, 100, 125, 150, 175, 200, 300, 400, 500, or 1000 times more selective for binding to the NMDA receptor than a calcium channel.

Screening compounds to determine the affinity of a drug for a particular receptor is one of the critical in the drug discovery process. Processes to determine receptor selectivity can be done by any method known to one skilled in the art. Screening can be used as a primary screening method for large compound libraries or as a secondary screen to rank compounds for binding affinity for various receptor types or subtypes. In one embodiment, this analysis can be done in a high throughput system, for example, filter-plate screening systems, such as the Millipore Multiscreen™_{HTS} filter plate.

In one embodiment, radioligand binding assays can be used to determine the receptor selectivity for a particular receptor. In one particular embodiment, saturation binding assays can be used to determine the binding constant (K_{d}) of a test compound for a particular receptor. Saturation binding assays can be performed according to any method known in the art. In general, saturation binding assays can be conducted by obtaining a cell membrane that expresses a particular receptor. For example, a cell, such as a CHO cell, can be transfected to express a glutamate receptor, such as an NMDA receptor, for example, an NR1/NR2A or NR1/NR2B NMDA receptor, or an AMPA receptor. Alternatively, cells can be used that endogenously expresses a particular receptor, such as an NMDA receptor, for example, an NR1/NR2B or NR1/NR2A NMDA receptor. In one embodiment, whole cell binding assays can be conducted. Alternatively, membranes can be isolated from the cell, such as, for example, by lysing the cell and then using centrifugation to obtain the membrane fraction of the lysate, see, for example, Laboratory method for isolation of cell membranes, A. Hubbard and Z. Cohn The Journal of Cell Biology (1975) and Rogers et al., 1991, J. Neuroscience: 2713-2724. The whole cell or cell membranes can then be incubated with serial dilutions of radiolabeled ligand, i.e. test compound, for example 3H-labeled ligand. After incubation for a period of time, for example, at least 1, 2 or 3 hours, the membranes can be washed a number of times, for example, 5, 10, 15 or 20 times. Scintillation fluid can then be added and the cells or radioactivity of the cells or membranes can be conducted. Non-specific binding can also be determined in a separate experiment with an excess of unlabeled competitor ligand. Specific binding can be calculated as non-specific activity subtracted from total activity. Binding constants (Kd) can then be determined by fitting specific binding by free ligand concentration by non-linear regression and Scatchard analysis, for example by using Prizm data software (www.Graphpad.com). In addition, the number of binding sites [maximal binding capacities (Bₘₐₓ) can also be calculated by non-linear regression and Scatchard analysis, for example by using Prizm data software.

In another embodiment, displacement radioligand binging assays can be conducted to determine relative affinity values (IC₅₀). Whole cells expressing particular receptors or isolated cell membranes can be used, as described above. Inhibition can be determined by using a constant radioligand concentration and serial dilutions of unlabelled competitor ligand as compared to a control binding experiment without unlabelled ligand (% Control). Relative affinity values (IC₅₀) can be determined by fitting binding inhibition values by non-linear regression, for example, by using Prizm data software.

Selected Compounds According to the Invention

The following compounds have been selected for improved mammalian, for example, human, clinical performance in the treatment of a disorder that can be mediated by an NMDA-receptor antagonist. Other compounds can be selected that satisfy the new parameters by following the guidance described generally herein.

In one embodiment, the compound selected according to the processes and methods described herein can be:

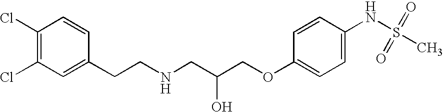

as well as pharmaceutically acceptable salts, enantiomers, enantiomeric mixtures, and mixtures.

In another embodiment, the compound selected according to the processes and methods described herein can be:

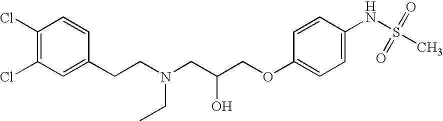

as well as pharmaceutically acceptable salts, enantiomers, enantiomeric mixtures, and mixtures.

In another embodiment, the compound selected according to the processes and methods described herein can be:

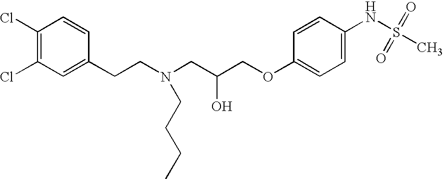

as well as pharmaceutically acceptable salts, enantiomers, enantiomeric mixtures, and mixtures.

In a further embodiment, the compound selected according to the processes and methods described herein can be:

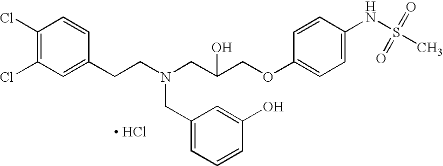

as well as pharmaceutically acceptable salts, enantiomers, enantiomeric mixtures, and mixtures.

In a still further embodiment, the compound selected according to the processes and methods described herein can be:

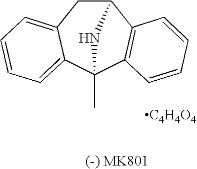

as well as pharmaceutically acceptable salts. In one embodiment, (−)MK801 can be used to treat neuropathic pain, brain tumors and/or neurodegenerative diseases as described herein.

In another embodiment, the compound (S) ketamine is not selected for the treatment of ischemic injury or hyopoxia. In another embodiment, (S) ketamine can be used to treat neuropathic pain, brain tumors and/or neurodegenerative diseases as described herein.

Synthesis of the MK801 (5S,10R)-(−)isomer can be achieved via racemic synthesis, followed by resolution to obtain the enantiomerically pure (−) isomer (see, for example, Molander, G. A., et al., J. Org. Chem., 64: pp. 6515-6517 (1999); Christy, M. E., et al., J. Org. Chem., 44: pp. 3117 (1979)), or via enantioselective synthesis in six steps from the Reissert product using regioselective radical cyclization (see, for example, Funabashi, K., et al., J. Am. Chem. Soc., 123: pp. 10784-10785 (2001)).

The syntheses of other compounds disclosed herein can be found in WO 02/072542.

Stereochemistry

It is appreciated that the three dimensional configuration of the compound may play a role in the activity and or suitability of the compound for therapeutic use. It has been observed experimentally herein that enantiomers of compounds may both be selected using the criteria described herein or one may be selected and one not selected. Presumably, in certain situations, both enantiomers may be selected using the providec criteria.

Nonlimiting examples are as follows.

In one embodiment, the enantiomer (S) 93-4, as indicated in FIG. 1, falls within the criteria for selection as an effective compound for therapeutic use.

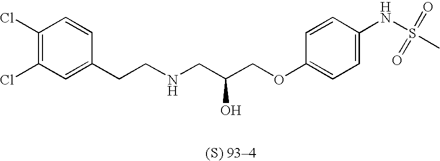

In anther embodiment, the enantiomer (S) 93-31, as indicated in FIG. 1, falls within the criteria for selection as an effective compound for therapeutic use.

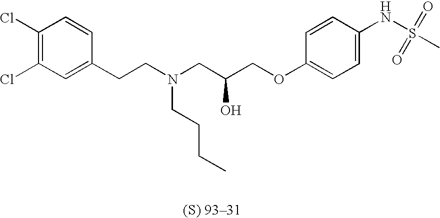

In anther embodiment, the enantiomer (S) 93-8, as indicated in FIG. 1, falls within the criteria for selection as an effective compound for therapeutic use.

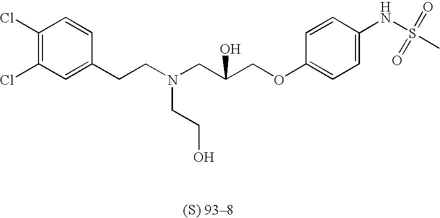

In anther embodiment, the enantiomer (S) 93-41, as indicated in FIG. 1, falls within the criteria for selection as an effective compound for therapeutic use.

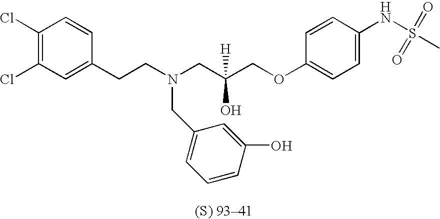

In yet another embodiment, the enantiomer (S) 93-5, as indicated in FIG. 1, falls within the criteria for selection as an effective compound for therapeutic use.

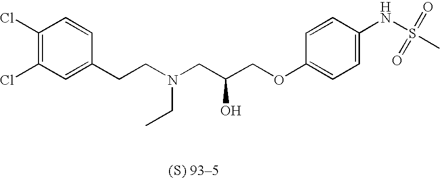

In one embodiment, compounds (S) 98-5, (S) 93-4, (S) 93-8, (S) 93-31 and (S) 93-41 can bind to the NR2B subunit of the NMDA receptor, for example as indicated in FIG. 1. In another embodiment, compounds (S) 98-5, (S) 93-4, (S) 93-8, (S) 93-31 and (S) 93-41 can be selective for the NR2B subunit of NMDA receptors.

In yet another embodiment, (−) MK801, as indicated in FIG. 1, falls within the criteria for selection as an effective compound for therapeutic use, yet (+)-MK801 does not fall within the criteria for selection as described herein.

Neither enantiomer 93-40 and 93-43, as indicated in FIG. 1, do not fall within the criteria for selection as an effective compound for therapeutic use.

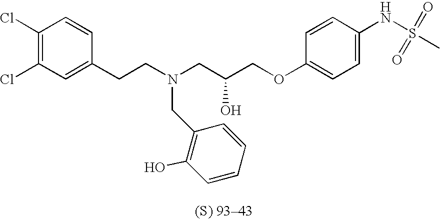

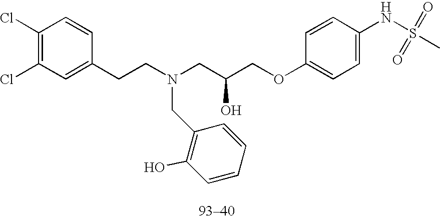

In addition, the enantiomer (S) 93-97, as indicated in FIG. 1, does not fall within the criteria for selection as an effective compound for therapeutic use.

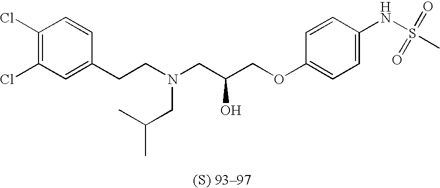

Additional Embodiments

In one embodiment, (S) ketamine can be specifically excluded from the methods of the present invention. In another embodiment, (−)MK801 can be specifically excluded from the methods of the present invention. In another embodiment, (S) ketamine can be excluded from the present invention with respect to treating an inschemic injury. In a further embodiment, (−) MK801 can be excluded from the present invention with respect to treating an inschemic injury.

In another embodiment, the compound selected according to the processes and methods described herein is not an NMDA receptor channel blocker, such as, but not limited to FR 115427, NPS 1506, phencyclidine (PCP), remacemide, TCP, or EAA-090. In another embodiment, the compound selected according to the processes and methods described herein is not an NMDA receptor glutamate site antagonist, such as, but not limited to, CGP 40116, D-CPPene, GPI3000 (NPC 17742), MDL 100,453, or selfotel (CGS 19755). In another embodiment, the compound selected according to the processes and methods described herein is not an NMDA receptor glycine site antagonist, such as, but not limited to 7-Cl-kynurenate, HA966, MRZ 2/576, ZD9379, gavestinel (GV150526), andlicostinel (ACEA 1021, 5-nitro-6,7-dichloro-1,4-dihydro-2,3-quinoxalinedione).

In another embodiment, the compound selected according to the processes and methods described herein is not described in PCT Publication No. WO 02/072542, including: 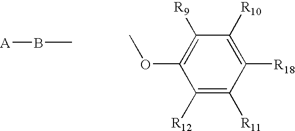
wherein one of R₉, R₁₀, R₁₁, R₁₂ and R₁₈ is 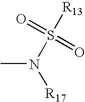
   where R₁₃ is alkyl, aralkyl or aryl; where R₁₇ is H or lower alkyl; and the others of R₉, R₁₀, R₁₁, R₁₂ and R₁₈ are H, F, Cl, I or R wherein R is lower alkyl; or: 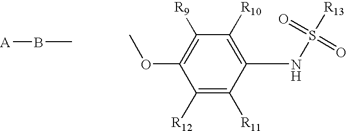
wherein R₉, R₁₀, R₁₁, and R₁₂ are independently selected from the group consisting of H, F, Cl, Br, I, and R wherein R is lower alkyl, and R₁₃ is alkyl aralkyl or aryl;
wherein A is selected from the group consisting of: 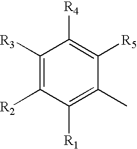
wherein R₁ and R₅ are independently H or F; R₂, R₃ and R₄ are independently selected from the group consisting of H, F, Cl, Br, I and OR where R is lower alkyl, or R₂ and R₃ taken together are O—CH₂—O; 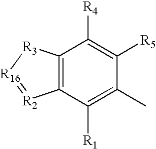
wherein R₁, R₄, and R₅ are independently selected from the group consisting of H, F, Cl, Br, I and OR where R is lower alkyl, R₃ is independently O, S, NH or NR, R₂ is N, and R₁₆ is C-alkyl, C-aralkyl or C-aryl; 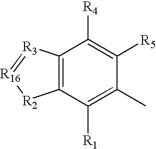
wherein R₁, R₄, and R₅ are independently selected from the group consisting of H, F, Cl, Br, I and OR where R is lower alkyl, R₂ is independently O, S, NH or NR, R₃ is N; and R₁₆ is C-alkyl, C-aralkyl or C-aryl; 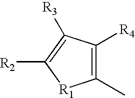
wherein R₁ through R₄ are independently selected from the group consisting of H, F, Cl, Br, I and OR where R is lower alkyl, or R₂ and R₃ taken together are O—CH₂—O; 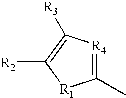
wherein R₁, R₂ and R₃ are independently selected from the group consisting of O, S, NH or NR where R is lower alkyl, or R₂ and R₃ taken together are O—CH₂—O, and R₄ is N; 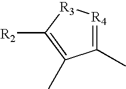
wherein R₂ and R₃ are independently selected from the group consisting of H, F, Cl, Br, I and OR where R is lower alkyl; and R₄ is N; 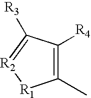
wherein R₁ is selected from the group consisting of O, S, NH and NR where R is lower alkyl; R₂ is N, and R₃ and R₄ are independently selected from the group consisting of H, F, Cl, Br, I and OR where R is lower alkyl; 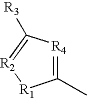
wherein R₁ is selected from the group consisting of O, S, NH and NR where R is lower alkyl; R₂ and R₄ are N, and R₃ is independently selected from the group consisting of H, F, Cl, Br, I and OR where R is lower alkyl; 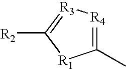
wherein R₁ is selected from the group consisting of O, S, NH and NR where R is lower alkyl; R₂ is selected from the group consisting of H, F, Cl, Br, I and OR where R is lower alkyl; and R₃ and R₄ are N; 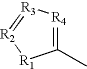
wherein R₁ is selected from the group consisting of O, S, NH and NR where R is lower alkyl; and R₂, R₃ and R₄ are N; 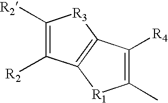
wherein R₁ and R₃ are independently selected from the group consisting of O, S, NH and NR where R is lower alkyl; and R₂, R₂′ and R₄ are independently selected from the group consisting of H, F, Cl, Br, I and OR where R is lower alkyl; 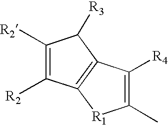
wherein R₁ and R₂ are independently selected from the group consisting of O, S, NH and NR where R is lower alkyl; and R₂′, and R₃ and R4 are independently selected from the group consisting of H, F, Cl, Br, I and OR where R is lower alkyl; 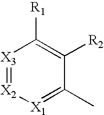
wherein X₁ is C—R₃ or N, X₂ is C—R₄ or N, X₃ is C—R₄′ or N where R₁-R₄′ are independently selected from the group consisting of O, S, NH and NR where R is lower alkyl, or where R₁ and R₂ taken together are O—CH₂—O;
and wherein B is selected from the group consisting of: 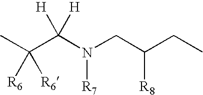
wherein R₆ and R₆′ are independently H or F; and R₇ is H, lower n-alkyl, CH₂Ar, CH₂CH₂Ar, CH₂CHFAr, or CH₂CHF₂Ar; and R₈ is OH, OR, where R is lower alkyl, or F; 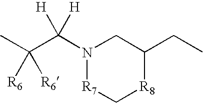
wherein R₆ and R₆′ are independently H or F; R₇ is CH₂ and R₈ is O; 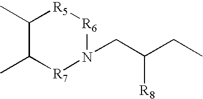
wherein R₅, R₆ and R₇ are independently CH₂, CHR or CR₂ where R is lower alkyl; and R₈ is OH, OR, where R is lower alkyl, or F; 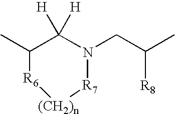
wherein R₆ and R₇ are independently CH₂, CHR or CR₂ where R is lower alkyl; and R₈ is OH, OR, where R is lower alkyl, or F; or 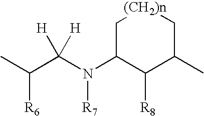
wherein R₆ and R₇ are independently CH₂, CHR or CR2 where R is lower alkyl; R₈ is OH or F; and n=1-3; and
pharmaceutically acceptable salts, enantiomers, enantiomeric mixtures, and mixtures of the foregoing.

The term “alkyl” takes its usual meaning in the art and is intended to include straight-chain, branched and cycloalkyl groups. The term includes, but is not limited to, methyl, ethyl, n-propyl, isopropyl, n-butyl, sec-butyl, isobutyl, tert-butyl, n-pentyl, neopentyl, 2-methylbutyl, 1-methylbutyl, 1-ethylpropyl, 1,1-dimethylpropyl, n-hexyl, 1-methylpentyl, 2-methylpentyl, 3-methylpentyl, 4-methylpentyl, 3,3-dimethylbutyl, 2,2-dimethylbutyl, 1,1-dimethylbutyl, 2-ethylbutyl, 1-ethylbutyl, 1,3-dimethylbutyl, n-heptyl, 5-methylhexyl, 4-methylhexyl, 3-methylhexyl, 2-methylhexyl, 1-methylhexyl, 3-ethylpentyl, 2-ethylpentyl, 1-ethylpentyl, 4,4-dimethylpentyl, 3,3-dimethylpentyl, 2,2-dimethylpentyl, 1,1-dimethylpentyl, n-octyl, 6-methylheptyl, 5-methylheptyl, 4-methylheptyl, 3-methylheptyl, 2-methylheptyl, 1-methylheptyl, 1-ethylhexyl, 1-propylpentyl, 3-ethylhexyl, 5,5-dimethylhexyl, 4,4-dimethylhexyl, 2,2-diethylbutyl, 3,3-diethylbutyl, and 1-methyl-1-propylbutyl. Alkyl groups are optionally substituted. Lower alkyl groups include among others methyl, ethyl, n-propyl, and isopropyl groups. Lower alkyl groups as referred to herein have one to six carbon atoms.

The term “bulky ring-containing group” refers to a group containing 1 or more ring structures which may be aryl rings or cycloalkyl rings.

The term “cycloalkyl” refers to alkyl groups having a hydrocarbon ring, particularly to those having rings of 3 to 7 carbon atoms. Cycloalkyl groups include those with alkyl group substitution on the ring. Cycloalkyl groups can include straight-chain and branched-chain portions. Cycloalkyl groups include but are not limited to cyclopropyl, cyclobutyl, cyclopentyl, cyclohexyl, cycloheptyl, cyclooctyl, and cyclononyl. Cycloalkyl groups can optionally be substituted.

The term “aryl” is used herein generally to refer to aromatic groups which have at least one ring having a conjugated pi electron system and includes without limitation carbocyclic aryl, aralkyl, heterocyclic aryl, biaryl groups and heterocyclic biaryl, all of which can be optionally substituted. Particular aryl groups have one or two aromatic rings.

Substitution of alkyl groups includes substitution at one or more carbons in the group by moieties containing heteroatoms. Suitable substituents for these groups include but are not limited to OH, SH, NH₂, COH, CO₂H, ORc, SRc, NRc Rd, CONRc Rd, and halogens, particularly fluorines where Rc and Rd, independently, are alkyl, unsaturated alkyl or aryl groups. Particular alkyl and unsaturated alkyl groups are lower alkyl, alkenyl or alkynyl groups having from 1 to about 3 carbon atoms.

“Aralkyl” refers to an alkyl group substituted with an aryl group. Suitable aralkyl groups include among others benzyl, phenethyl and picolyl, and may be optionally substituted. Aralkyl groups include those with heterocyclic and carbocyclic aromatic moieties.

“Heterocyclic aryl groups” refers to groups having at least one heterocyclic aromatic ring with from 1 to 3 heteroatoms in the ring, the remainder being carbon atoms. Suitable heteroatoms include without limitation oxygen, sulfur, and nitrogen. Heterocyclic aryl groups include among others furanyl, thienyl, pyridyl, pyrrolyl, N-alkyl pyrrolo, pyrimidyl, pyrazinyl, imidazolyl, benzofuranyl, quinolinyl, and indolyl, all optionally substituted.

“Heterocyclic biaryl” refers to heterocyclic aryls in which a phenyl group is substituted by a heterocyclic aryl group ortho, meta or para to the point of attachment of the phenyl ring to the decalin or cyclohexane. Para or meta substitution is useful. Heterocyclic biaryl includes among others groups which have a phenyl group substituted with a heterocyclic aromatic ring. The aromatic rings in the heterocyclic biaryl group can be optionally substituted.

“Biaryl” refers to carbocyclic aryl groups in which a phenyl group is substituted by a carbocyclic aryl group ortho, meta or para to the point of attachment of the phenyl ring to the decalin or cyclohexane. Biaryl groups include among others a first phenyl group substituted with a second phenyl ring ortho, meta or para to the point of attachment of the first phenyl ring to the decalin or cyclohexane structure. Para substitution is useful. The aromatic rings in the biaryl group can be optionally substituted.

Aryl group substitution includes substitutions by non-aryl groups (excluding H) at one or more carbons or where possible at one or more heteroatoms in aromatic rings in the aryl group. Unsubstituted aryl, in contrast, refers to aryl groups in which the aromatic-ring carbons are all substituted with H, e.g. unsubstituted phenyl(—C₆H₅), or naphthyl(—C₁₀H₇). Suitable substituents for aryl groups include among others alkyl groups, unsaturated alkyl groups, halogens, OH, SH, NH₂, COH, CO₂H, Ore, Sre, Nre Rf, CONRe Rf, where Re and Rf independently are alkyl, unsaturated alkyl or aryl groups. Particular substituents are OH, SH, Ore, and Sre where Re is a lower alkyl, i.e. an alkyl group having from 1 to about 3 carbon atoms. Other particular substituents are halogens, more preferably fluorine, and lower alkyl and unsaturated lower alkyl groups having from 1 to about 3 carbon atoms. Substituents include bridging groups between aromatic rings in the aryl group, such as —CO₂—, —CO—, —O—, —S—, —NH—, —CHCH— and —(CH₂)₁— where 1 is an integer from 1 to about 5, and particularly —CH₂—. Examples of aryl groups having bridging substituents include phenylbenzoate, Substituents also include moieties, such as —(CH₂)₁—, —O—(CH₂)¹— or —OCO—(CH₂)₁—, where 1 is an integer from about 2 to 7, as appropriate for the moiety, which bridge two ring atoms in a single aromatic ring as, for example, in a 1,2,3,4-tetrahydronaphthalene group. Alkyl and unsaturated alkyl substituents of aryl groups can in turn optionally be substituted as described supra for substituted alkyl and unsaturated alkyl groups.

In an alternative embodiment, the compound selected according to the processes and methods described herein is not:

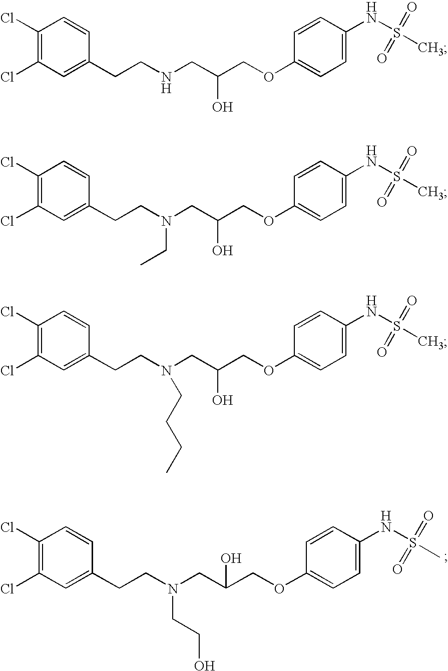

or in another embodiment, pharmaceutically acceptable salts, esters, enantiomers, enantiomeric mixtures, and mixtures thereof.

In another alternative embodiment, the compound selected according to the processes and methods described herein is not:

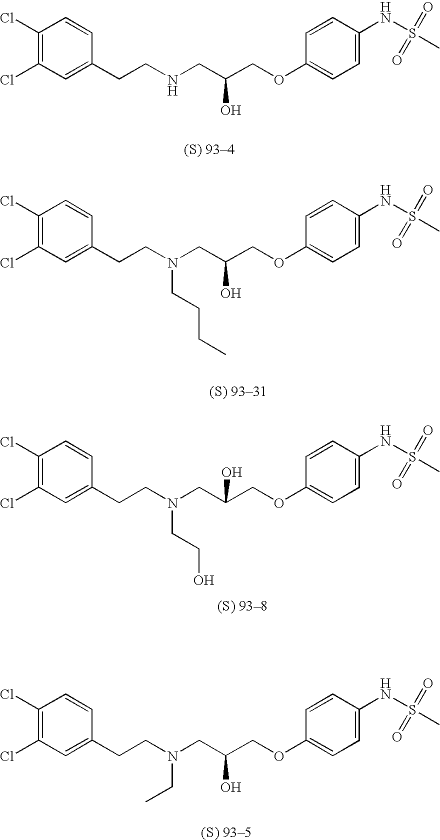

or in another embodiment, pharmaceutically acceptable salts, esters, and mixtures thereof.

Side Effects

In an additional aspect of the methods and processes described herein, the compound does not exhibit substantial toxic an/or psychotic side effects. Toxic side effects include, but are not limited to, agitation, hallucination, confusion, stupor, paranoia, delirium, psychotomimetic-like symptoms, rotarod impairment, amphetamine-like stereotyped behaviors, stereotypy, psychosis memory impairment, motor impairment, anxiolytic-like effects, increased blood pressure, decreased blood pressure, increased pulse, decreased pulse, hematological abnormalities, electrocardiogram (ECG) abnormalities, cardiac toxicity, heart palpitations, motor stimulation, psychomotor performance, mood changes, short-term memory deficits, long-term memory deficits, arousal, sedation, extrapyramidal side-effects, ventricular tachycardia. Lengthening of cardiac repolarisation, ataxia, cognitive deficits and/or schizophrenia-like symptoms.

Further, in another embodiment, the compounds selected or identified according to the processes and methods described herein do not have substantial side effects associated with other classes of NMDA receptor antagonists. In one embodiments, such compounds do not substantially exhibit the side effects associated with NMDA antagonists of the glutamate site, such as selfotel, D-CPPene (SDZ EAA 494) and AR-R15896AR (ARL 15896AR), including, agitation, hallucination, confusion and stupor (Davis et al. (2000) Stroke 31(2):347-354; Diener et al. (2002), J Neurol 249(5):561-568); paranoia and delirium (Grotta et al. (1995), J Intern Med 237:89-94); psychotomimetic-like symptoms (Loscher et al. (1998), Neurosci Lett 240(1):33-36); poor therapeutic ratio (Dawson et al. (2001), Brain Res 892(2):344-350); amphetamine-like stereotyped behaviors (Potschka et al. (1999), Eur J Pharmacol 374(2):175-187). In another embodiment, such compounds do not exhibit the side effects associated with NMDA antagonists of the glycine site, such as HA-966, L-701,324, d-cycloserine, CGP-40116, and ACEA 1021, including significant memory impairment and motor impairment (Wlaz, P (1998), Brain Res Bull 46(6):535-540). In a still further embodiment, such compounds do not exhibit the side effects of NMDA high affinity receptor channel blockers, such as MK-801 and ketamine, including, psychosis-like effects (Hoffman, D C (1992), J Neural Transm Gen Sect 89:1-10); cognitive deficits (decrements in free recall, recognition memory, and attention; Malhotra et al (1996), Neuropsychopharmacology 14:301-307); schizophrenia-like symptoms (Krystal et al (1994), Arch Gen Psychiatry 51:199-214; Lahti et al. (2001), Neuropsychopharmacology 25:455-467), and hyperactivity and increased stereotpy (Ford et al (1989) Physiology and behavior 46: 755-758.

In a further additional or alternative embodiment, the compound has a therapeutic index equal to or greater than at least 2:1, at least 3:1, at least 4:1, at least 5:1, at least 6:1, at least 7:1, at least 8:1, at least 9:1, at least 10:1, at least 15:1, at least 20:1, at least 25:1, at least 30:1, at least 40:1, at least 50:1, at least 75:1, at least 100:1 or at least 1000:1. The therapeutic index can be defined as the ratio of the dose required to produce toxic or lethal effects to dose required to produce therapeutic responses. It can be the ratio between the the median toxic dose (the dosage at which 50% of the group exhibits the adverse effect of the drug) and the median effective dose (the dosage at which 50% of the population respond to the drug in a specific manner). The higher the therapeutic index, the more safe the drug is considered to be. It simply indicates that it would take a higher dose to invoke a toxic response that it does to cause a beneficial effect.

The side effect profile of compounds can be determined by any method known to those skilled in the art. In one embodiment, motor impairment can be measured by, for example, measuring locomotor activity and/or rotorod performance. Rotorod experiments involve measuring the duration that an animal can remain on an accelerating rod. In another embodiment, memory impairment can be assessed, for example, by using a passive avoidance paradigm; Sternberg memory scanning and paired words for short-term memory, or delayed free recall of pictures for long-term memory. In a further embodiment, anxiolytic-like effects can be measured, for example, in the elevated plus maze task. In other embodiments, cardiac function can be monitored, blood pressure and/or body temperature measured and/or electrocardiograms conducted to test for side effects. In other embodiments, psychomotor functions and arousal can be measured, for example by analyzing critical flicker fusion threshold, choice reaction time, and/or body sway. In other embodiments, mood can be assessed using, for example, self-ratings. In further embodiments, schizophrenic symptoms can be evaluated, for example, using the PANSS, BPRS, and CGI, side-effects were assessed by the HAS and the S/A scale.

IV. Diseases

In additional aspects of the present invention, methods are provided to treat patients by administering a compound selected according to the methods or processes described herein. Any disease, condition or disorder which induces a low pH can be treated according to the methods described herein.

Further provided are methods to attenuate the progression of an ischemic, hypoxic or excitotoxic cascade associated with a drop in pH by administering an effective amount of a compound that exhibits the properties described herein. In addition, methods are provided to decrease infarct volume associated with a drop in pH by administering a compound that exhibits the properties described herein. Further, a method is provided to decrease cell death associated with a drop in pH by administering a compound that exhibits the properties described herein. Still further, methods are provided to decrease behavioral deficits associated with an ischemic event associated with a drop in pH by administering a compound that exhibits the properties described herein.

In one embodiment, methods are provided to treat patients with ischemic injury or hypoxia, or prevent or treat the neuronal toxicity associated with ischemic injury or hypoxia, by administering a compound selected according to the methods or processes described herein. In one particular embodiment, the ischemic injury can be stroke. In other embodiments, the ischemic injury can be selected from, but not limited to, one of the following: traumatic brain injury, cognitive deficit after bypass surgery, cognitive deficit after carotid angioplasty; and/or neonatal ischemia following hypothermic circulatory arrest.

In another particular embodiment, the ischemic injury can be vasospasm after subarachnoid hemorrhage. A subarachnoid hemorrhage refers to an abnormal condition in which blood collects beneath the arachnoid mater, a membrane that covers the brain. This area, called the subarachnoid space, normally contains cerebrospinal fluid. The accumulation of blood in the subarachnoid space and the vasospasm of the vessels which results from it can lead to stroke, seizures, and other complications. The methods and compounds described herein can be used to treat patients experiencing a subarachnoid hemorrhage. In one embodiment, the methods and compounds described herein can be used to limit the toxic effects of the subarachnoid hemorrhage, including, for example, stroke and/or ischemia that can result from the subarachnoid hemorrhage. In a particular embodiment, the methods and compounds described herein can be used to treat patients with traumatic subarachnoid hemorrhage. On one embodiment, the traumatic subarachnoid hemorrhage can be due to a head injury. In another embodiment, the patients can have a spontaneous subarachnoid hemorrhage.

In another embodiment, methods are provided to treat patients with neuropathic pain or related disorders by administering a compound selected according to the methods or processes described herein. In certain embodiments, the neuropathic pain or related disorder can be selected from the group including, but not limited to: peripheral diabetic neuropathy, postherpetic neuralgia, complex regional pain syndromes, peripheral neuropathies, chemotherapy-induced neuropathic pain, cancer neuropathic pain, neuropathic low back pain, HIV neuropathic pain, trigeminal neuralgia, and/or central post-stroke pain.

Neuropathic pain can be associated with signals generated ectopically and often in the absence of ongoing noxious events by pathologic processes in the peripheral or central nervous system. This dysfunction can be associated with common symptoms such as allodynia, hyperalgesia, intermittent abnormal sensations, and spontaneous, burning, shooting, stabbing, paroxysmal or electrical-sensations, paresthesias, hyperpathia and/or dysesthesias, which can also be treated by the compounds and methods described herein.

Further, the compounds and methods described herin can be used to treat neuropathic pain resulting from peripheral or central nervous system pathologic events, including, but not limited to trauma, ischemia; infections or from ongoing metabolic or toxic diseases, infections or endocrinologic disorders, including, but not limited to, diabetes mellitus, diabetic neurophathy, amyloidosis, amyloid polyneuropathy (primary and familial), neuropathies with monoclonal proteins, vasculitic neuropathy, HIV infection, herpes zoster—shingles and/or postherpetic neuralgia; neuropathy associated with Guillain-Barre syndrome; neuropathy associated with Fabry's disease; entrapment due to anatomic abnormalities; trigeminal and other CNS neuralgias; malignancies; inflammatory conditions or autoimmune disorders, including, but not limited to, demyelinating inflammatory disorders, rheumatoid arthritis, systemic lupus erythematosus, Sjogren's syndrome; and cryptogenic causes, including, but not limited to idiopathic distal small-fiber neuropathy. Other causes of neuropathic pain that can be treated according to the methods and compositions described herein include, but are not limited to, exposure to toxins or drugs (such as aresnic, thallium, alcohol, vincristine, cisplatinum and dideoxynucleosides), dietary or absorption abnormalities, immuno-globulinemias, hereditary abnormalities and amputations (including mastectomy). Neuropathic pain can also result from compression of nerve fibers, such as radiculopathies and carpal tunnel syndrome.

In another embodiment, methods are provided to treat patients with brain tumors by administering a compound selected according to the methods or processes described herein. In a further embodiment, methods are provided to treat patients with neurodegenerative diseases by administering a compound selected according to the methods or processes described herein. In one embodiment, the neurodegenerative disease can be Parkinson's disease. In another embodiment, the neurodegenerative disease can be Alzheimer's, Huntington's and/or Amyotrophic Lateral Sclerosis.

Further, compounds selected according to the methods or processes described herein can be used prophylactically to prevent or protect against such diseases or neurological conditions, such as those described herein. In one embodiment, patients with a predisposition for an ischemic event, such as a genetic predisposition, can be treated prophylactically with the methods and compounds described herein. In another embodiment, patients that exhibit vasospasms can be treated prophylactically with the methods and compounds described herein. In further embodiment, patients that have undergone cardiac bypass surgery can be treated prophylactically with the methods and compounds described herein.

In addition, methods are provided to treat the following diseases or neurological conditions, including, but not limited to: chronic nerve injury, chronic pain syndromes, such as, but not limited to diabetic neuropathy, ischemia, ischemia following transient or permanent vessel occlusion, seizures, spreading depression, restless leg syndrome, hypocapnia, hypercapnia, diabetic ketoacidosis, fetal asphyxia, spinal cord injury, traumatic brain injury, status epilepticus, epilepsy, hypoxia, perinatal hypoxia, concussion, migraine, hypocapnia, hyperventilation, lactic acidosis, fetal asphyxia during parturition, brain gliomas, and/or retinopathies by administering a compound selected according to the methods or processes described herein.

V. Administration/Formulations

Hosts, including mammals and particularly humans, suffering from any of the disorders described herein, can be treated by administering to the host an effective amount of a compound described herein, or a pharmaceutically acceptable prodrug, ester, and/or salt thereof, optionally in combination with a pharmaceutically acceptable carrier or diluent. The active compounds can be administered by any appropriate route, for example, orally, parenterally, intravenously, intradermally, intramuscularly, subcutaneously, sublingually, transdermally, bronchially, pharyngolaryngeal, intranasally, topically such as by a cream or ointment, rectally, intraarticular, intracisternally, intrathecally, intravaginally, intraperitoneally, intraocularly, by inhalation, bucally or as an oral or nasal spray.

The compounds of the present invention can be used in the form of pharmaceutically acceptable salts derived from inorganic or organic acids. By “pharmaceutically acceptable salt” is meant those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, P. H. Stahl, et al. describe pharmaceutically acceptable salts in detail in “Handbook of Pharmaceutical Salts: Properties, Selection, and Use” (Wiley VCH, Zürich, Switzerland: 2002). The salts can be prepared in situ during the final isolation and purification of the compounds of the present invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxyethansulfonate(isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like.

Pharmaceutically acceptable salts may be also obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium or magnesium) salts of carboxylic acids can also be made.

The formulations may conveniently be presented in unit dosage form and may be prepared by any of the methods well known in the art of pharmacy. All methods include the step of bringing into association a compound of the invention or a pharmaceutically acceptable salt or solvate thereof (“active compound”) with the carrier which constitutes one or more accessory compounds. In general, the formulations are prepared by uniformly and intimately bringing into association the active compound with liquid carriers or finely divided solid carriers or both and then, if necessary, shaping the product into the desired formulation.

The compound or a pharmaceutically acceptable ester, salt, solvate or prodrug can be mixed with other active materials that do not impair the desired action, or with materials that supplement the desired action. Solutions or suspensions used for parenteral, intradermal, subcutaneous, or topical application can include, for example, the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic. Administered intravenously, particular carriers are physiological saline or phosphate buffered saline (PBS).

Pharmaceutical compositions of this invention for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants.

These compositions may also contain adjuvants including preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form may be brought about by the use of agents delaying absorption, for example, aluminum monostearate and gelatin.

In some cases, in order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Suspensions, in addition to the active compounds, may contain suspending agents, as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

Besides inert diluents, the formulation compositions can also include adjuvants such as wetting agents, emulsifying and suspending agents, sweetening, flavoring, and perfuming agents.

The active compounds can also be in micro-or nano-encapsulated form, if appropriate, with one or more excipients.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use. Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Formulations for parenteral (including subcutaneous, intradermal, intramuscular, intravenous and intraarticular) administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example, saline, water-for-injection, immediately prior to use. Extemporaneous injection solutions and suspensions may be prepared from sterile powders, granules and tablets of the kind previously described.

Another method of formulation of the present invention involves conjugating the compounds described herein to a polymer that enhances aqueous solubility. Examples of suitable polymers include but are not limited to polyethylene glycol, poly-(d-glutamic acid), poly-(1-glutamic acid), poly-(1-glutamic acid), poly-(d-aspartic acid), poly-(1-aspartic acid), poly-(1-aspartic acid) and copolymers thereof. Polyglutamic acids having molecular weights between about 5,000 to about 100,000 can be used, with molecular weights between about 20,000 and 80,000 can be used and with molecular weights between about 30,000 and 60,000 can also be used. The polymer is conjugated via an ester linkage to one or more hydroxyls of an inventive epothilone using a protocol as essentially described by U.S. Pat. No. 5,977,163 which is incorporated herein by reference. Particular conjugation sites include the hydroxyl off carbon-21 in the case of 21-hydroxy-derivatives of the present invention. Other conjugation sites include but are not limited to the hydroxyl off carbon 3 and/or the hydroxyl off carbon 7.

In yet another formulation method, the inventive compounds can be conjugated to a monoclonal antibody. This strategy allows the targeting of the inventive compounds to specific targets. General protocols for the design and use of conjugated antibodies are described in “Monoclonal Antibody-Based Therapy of Cancer” [by Michael L. Grossbard, ed. (1998)].

The amount of active compound that may be combined with the carrier materials to produce a single dosage form will vary depending upon the subject treated and the particular mode of administration. For example, a formulation for intravenous use can comprise an amount of an inventive compound ranging from about 1 mg/mL to about 25 mg/mL, preferably from about 5 mg/mL to 15 mg/mL, and more preferably about 10 mg/mL. In accordance with the compositions of the present invention, a dose range of from about 0.001 mg/kg per day to about 2500 mg/kg per day is typical. Preferably, the dose range is from about 0.1 mg/kg per day to about 1000 mg/kg per day. More preferably, the dose range is from about 0.1 mg/kg per day to about 500 mg/kg per day, including 1 mg/kg, 2 mg/kg, 5 mg/kg, 10 mg/kg, 15 mg/kg, 20 mg, kg, 25 mg/kg, 30 mg/kg, 35 mg/kg, 40 mg/kg, 45 mg/kg, 50 mg/kg, 100 mg/kg, 200 mg/kg, 300 mg/kg, 400 mg/kg, 500 mg/kg per day, and values between any two of the values given in this range. The dose range for humans is generally from about 0.005 mg to 100 g/day. Alternatively, the dose range in accordance with the present invention is such that the blood serum level of compounds of the present invention is from about 0.01 μM to about 100 μM, and preferably from about 0.1 μM to about 100 μM. Suitable values of blood serum levels in accordance with the present invention include but are not limited to about 0.01 μM, about 0.1 μM, about 0.5 μM, about 1 μM, about 5 μM, about 10 μM, about 15 μM, about 20 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM and about 100 μM, as well as any blood serum level that falls within any two of these values (e.g, between about 10 μM and about 60 μM). Tablets or other forms of dosage presentation provided in discrete units may conveniently contain an amount of one or more of the compounds of the invention which are effective at such dosage ranges, or ranges in between these ranges.

Dosage Forms

The compounds and formulations of the present invention can be administered in any of the known dosage forms standard in the art; in solid dosage form, semi-solid dosage form, or liquid dosage form, as well as subcategories of each of these forms.

Solid dosage forms for oral administration include capsules, caplets, tablets, pills, powders, lozenges, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, capsules, pills, and granules can be prepared with coatings and shells such as enteric coatings and other coatings well known in the pharmaceutical formulating art. They may optionally contain opacifying agents and can also be of a composition that they release the active compound(s) only, or preferentially, in a certain part of the intestinal tract in a delayed manner. Examples of embedding compositions which can be used include polymeric substances and waxes.

A tablet may be made by compression or molding, optionally with one or more accessory compounds. Compressed tablets may be prepared by compressing in a suitable machine the active compound in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active compound therein.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Semi-liquid dosage forms include those dosage forms that are too soft in structure to qualify for solids, but to thick to be counted as liquids. These include creams, pastes, ointments, gels, lotions, and other semisolid emulsions containing the active compound of the present invention.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Formulations containing compounds of the invention may be administered through the skin by an appliance such as a transdermal patch. Patches can be made of a matrix such as polyacrylamide, polysiloxanes, or both and a semi-permeable membrane made from a suitable polymer to control the rate at which the material is delivered to the skin. Other suitable transdermal patch formulations and configurations are described in U.S. Pat. Nos. 5,296,222 and 5,271,940, as well as in Satas, D., et al, “Handbook of Pressure Sensitive Adhesive Technology, 2^{nd} Ed.”, Van Nostrand Reinhold, 1989: Chapter 25, pp. 627-642.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons. Such excipients are described, for example, in “Handbook of Pharmaceutical Excipients, 3^{rd} Ed.”, A. H. Kibbe, Ed. (American Pharmaceutical Association and Pharmaceutical Press, Washington, D.C., 2000), the entire contents of which are included herein by reference.

Controlled-Release Formulations

In one embodiment, the active compounds of the present invention are prepared with carriers that will protect the compound against rapid elimination from the body or rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylacetic acid. Methods for preparation of such formulations will be apparent to those skilled in the art.

The following examples are offered by way of illustration and not by way of limitation.

### EXAMPLES

### Example 1

### Selectivity of Compound 93-4 for NMDA Receptors Versus Other Glutamate Receptors

Compound 93-4 series was shown to be selective for NMDA receptors by lack of effects on Xenopus oocytes injected with AMPA receptor and kainate receptor subunits. Glutamate or domoate induced current recordings were performed using two electrode voltage clamp, and 3 uM of Compound 93-4 coadministered with agonist (glutamate for AMPA receptors, domoate for kainate receptors). No reduction in the agonist induced response was seen, indicating that Compound 93-4 does not inhibit AMPA and kainate receptors. In addition, 3 uM of Compound 93-4 was effective at inhibiting NMDA receptor mediated currents when receptors are comprised of NR1/NR2B subunits but not NR1/NR2A or NR1/NR2D receptors.

### Example 2

### Effects of 93 Series Compounds on Locomotor Activity of Rats

100-150 gm Sprague-Dawley rats were injected IP with varying doses of 93-4, 93-5, 93-8, 93-31, 93-40, 93-41 after 1 hour habituation in an activity box equipped with optical monitors to quantify locomotor activity as light beam breaks. Locomotor activity was monitored after injection for 2 hours. Both stereoisomers of MK801 were used as a positive control. (+)MK801 showed stereotypical biphasic effects on locomotor activity, with an initial increase in locomotor activity followed by a decrease that reflected ataxia. The data illustrate that (−) MK801 is at least 10-fold less potent than (+)MK801 in causing the induction of locomotor activity compared to vehicle injected control animals. In addition, 3-300 mg/kg 93-4, 3-300 mg/kg 93-5, 30-300 mg/kg of 93-8, 3-300 mg/kg of 93-31 (FIG. 5), 30 mg/kg of 93-40, and 30-300 mg/kg of 93-41 had no significant effects on locomotor activity. Doses of 93 series compounds known to be neuroprotective do not have effects on locomotor activity.

### Example 3

### Determination of pH Dependent Potency Shift in Xenopus oocytes

Expression of NMDA receptors in Xenopus oocytes. cRNA was synthesized from linearized template cDNA for NMDA receptor subunits (NR1-1a, NR2B, NR2A) according to manufacturer specifications (Ambion:). cDNAs used corresponded to GenBank numbers U08261 and U11418 (NR1-1a), AF001423 and CD13211 (NR2A), U11419 (NR2B). Briefly, cDNA was linearized with an appropriate restriction enzyme downstream of the coding region, purified, and incubated with RNA polymerase and appropriate concentrations of ribonucleotides. In vitro transcribed cRNA was purified using standard methods. Quality of synthesized cRNA was assessed by gel electrophoresis, and quantity was estimated by spectroscopy and gel electrophoresis. Stage V and VI oocytes were surgically removed from the ovaries of large, well-fed and healthy Xenopus laevis anesthetized with 3-amino-benzoic acid ethyl ester (1 gm/l). Clusters of oocytes were incubated with 292 U/ml Worthington (Freehold, N.J.) type IV collagenase or 1.3 mg/ml collagenase (Life Technologies, Gaithersburg, Md.; 17018-029) for 2 hr in Ca2+-free solution comprised of (in mM) 115 NaCl, 2.5 KCl, and 10 HEPES, pH 7.5, with slow agitation to remove the follicular cell layer. Oocytes were then washed extensively in the same solution supplemented with 1.8 mM CaCl2 and maintained in Barth's solution comprised of (in mM): 88 NaCl, 1 KCl, 24 NaHCO3, 10 HEPES, 0.82 MgSO4, 0.33 Ca(NO3)2, and 0.91 CaCl2 and supplemented with 100 ug/ml gentamycin, 40 ug/ml streptomycin, and 50 ug/ml penicillin. Oocytes were manually defolliculated and injected within 24 hr of isolation with 5 ng of NR1 subunit and 10 ng of NR2 subunit in a 50 nl volume, and incubated in Barth's solution at 18° C. for 3-7 d. Glass injection pipettes had tip sizes ranging from 10-20 microns, and were backfilled with mineral oil.

Preparation of pH-dependent NMDA receptor antagonists for testing NMDA receptor antagonists were typically made up as 20 mM solutions in 100% DMSO and stored at −20 C. This stock solution was sequentially diluted (1/10 v/v) to 2 mM, 0.2 mM, and 0.02 mM, all in 100% DMSO. These stock solutions were subsequently diluted to the appropriate concentration range in a working solution comprised of 90 mM NaCl, 3 mM KCl, 5 mM HEPES, 0.5 mM BaCl2, 10 uM EDTA, 100 uM glutamate, 50 uM glycine (pH either 6.9 or 7.6 adjusted with NAOH or HCl as appropriate). The concentrations of drug tested were 0.01, 0.03 micromolar (diluting 0.02 mM stock into appropriate volumes), 0.1, 0.3 micromolar (diluting 0.2 mM stock into appropriate volumes), 1, 3 micromolar (diluting 2 mM stock into appropriate volumes), and/or 10, 30, 100 micromolar (diluting 20 mM stock into appropriate volumes).

Voltage-clamp recordings from Xenopus oocytes. Two electrode voltage-clamp recordings were made 2-7 days post-injection. Oocytes were placed in a dual-track plexiglass recording chamber with a single perfusion line that splits in a Y-configuration to perfuse two oocytes. Dual recordings were made at room temperature using two Warner OC725B two-electrode voltage clamp amplifiers, arranged as recommended by the manufacturer. Glass microelectrodes (1-10 Megaohms) were filled with 300 mM KCl (voltage electrode) or 3 M KCl (current electrode). The bath clamps communicated across silver chloride wires placed into each side of the recording chamber, both of which were assumed to be at a reference potential of 0 mV. Oocytes were perfused with a solution comprised of (in mM) 90 NaCl, 1 KCl, 10 HEPES, and 0.5 BaCl2, pH 7.3, and held at −40 mV. Final concentrations for control application of glutamate (100 micromolar) plus glycine (50 micromolar) were achieved by adding appropriate volume from 100 and 30 mM stock solutions, respectively. In addition, 10 micromolar final EDTA was obtained by adding a 1:1000 dilution of 10 mM EDTA, in order to chelate contaminant divalent ions such as Zn2+. External pH was adjusted to either 6.9 or 7.6. Dose response curves were obtained by applying in successive fashion maximal glutamate and glycine, followed by glutamate/glycine plus variable concentrations of antagonist. Dose response curves consisting of 4 to 6 concentrations were obtained in this manner. The baseline leak current at −40 mV was measured before and after recording, and the full recording linearly corrected for any change in leak current. Oocytes with glutamate-evoked responses smaller than 100 nA at pH 7.6 or 50 nA at pH 6.9 were not included. The level of inhibition by applied antagonist was expressed as a percent of the initial glutamate response, and averaged together across oocytes from a single frog. Each experiment consisted of recordings at each pH from 3 to 10 oocytes obtained from a single frog. The average percent responses at each of 4 to 8 antagonist concentrations were fitted by the logistic equation, (100−min)/(1+([conc]/IC50)^{nH})+min, where min is the residual percent response in saturating antagonist, IC50 is the concentration of antagonist that causes half of the achievable inhibition, and nH is a slope factor describing steepness of the inhibitory curve. Min was constrained to be greater than or equal to 0. For experiments with known channel blockers, min was set to 0. The IC50 values obtained at pH 7.6 and 6.9 were expressed as a ratio and averaged together to determine the mean shift in IC50.

### Example 4

### Determination of Neuroprotection in an In Vivo Model of Transient Focal Ischemia

Transient Focal Ischemia Transient focal cerebral ischemia was induced by intraluminal middle cerebral artery (MCA) occlusion with a monofilament suture. Briefly, male C57BL/6 mice (3-5 months old, The Jackson Laboratory) were anesthetized with 2% isoflurane in 98% O2. The rectal temperature was controlled at 37° C. (range 36.5-37.5) with a homeothermic blanket. Relative changes in regional cerebral blood flow were monitored with a laser Doppler flowmeter (Perimed). To do this the probe was glued directly to the skull 2 mm posterior and 4-6 mm lateral of the bregma. An 11-mm 5-0 Dermalon or Look (SP185) black nylon non-absorbable suture with the tip flame-rounded was introduced into the left internal carotid artery through the external carotid artery stump until monitored blood flow was stopped (at 10.5-11 mm of suture insertion). After 30-min MCA occlusion, blood flow was restored by withdrawing the suture. After 24 hour survival, the brain was removed and cut into 2 mm sections. The lesion was identified with 2% 2,3,5-triphenyltetrazolium chloride (TTC) in PBS at 37° C. for 20 min. The infarct area of each section was measured using NIH IMAGE (Scion Corporation, Beta 4.0.2 release) and multiplied by the section thickness to give the infarct volume of that section. The density slice option in NIH IMAGE was used to segment the images based on the intensity determined as 70% or 75% of that in the contralateral undamaged cortex. This standard was maintained throughout the analysis in all animals, and only objects at this intensity were highlighted for area measurement. The area of the lesion, as identified by digitally identified threshold reductions in TTC staining, was manually outlined. A ratio of the contralateral to ipsilateral hemisphere section volume was multiplied by the corresponding infarct section volume to correct for edema. Infarct volume was determined by summing the infarct area times section thickness for all sections. At least 12 animals were included in each measurement. For some experiments, the regions of damage were directly measreud by circling freehand the region of reduced staining. Identical results were obtained with the two procedures.

Intraperitoneal administration of pH-dependent NMDA receptor antagonists. C57B1/6 mice received an intraperitoneal (IP) injection of 93-4, 93-5, 93-8, 93-31, 93-40 30 min before MCA occlusion surgery. A 30 mg/ml stock solution in 50% DMSO was prepared by adding 30 mg of compound into 0.5 ml of DMSO followed by addition of 0.5 ml of 0.9% saline with vortexing.

The working solution for the IP injection solution was 3 mg/ml in 0.9% saline (50% v/v DMSO), and was prepared by transferring 0.2 ml of the stock solution into a new tube and adding 0.9 ml of DMSO and 0.9 ml of 0.9% saline with vortexing. 3-30 mg/kg final dose was administered to mice, with injection volume varying depending on animal weight and desired dose.

Intracerebroventricular administration of pH-dependent NMDA receptor antagonists. In a separate set of experiments, mice received a small volume intracerebroventricular (ICV) injection of NMDA antagonist (93-5, 93-97, 93-31, 93-41, 93-43) or appropriate vehicle prior to surgery. Initially a 20 mM stock solution in 100% DMSO was prepared for all drugs. Five microliters of this stock solution was transferred to a new tube and 45 microliters of DMSO added for drugs 93-41, 93-43 with vortexing. 150 microliters of phosphate buffered saline (PBS, 0.9% NaCl, pH 7.4, Sigma 1000-3) was subsequently added to give a 0.5 mM drug solution in 25% (v/v) DMSO. For all other drugs, 5 microliters of 20 mM DMSO stock solution was transferred to a new tube and 15 ul of DMSO added with vortexing. To this solution 180 microliters of PBS was added to give a working solution of 0.5 mM drug in 10% v/v DMSO. For vehicle, DMSO was substituted for 20 mM drug in DMSO. All ICV injections were made into the right ventricle (2 mm posterior and 1 mm lateral of the bregma, needle inserted 3 mm) of male C57BL/6 mice (3-5 months old, The Jackson Laboratory) 30 min before MCA occlusion surgery. Mice were killed 24 h after MCA occlusion surgery and the lesion was identified and analyzed as described above. Mice with subarachnoid hemorrhage were identified by appearance of blood clot in excess of ˜1 mm at base of skull, and were excluded.

Results

Compounds 93-97, 93-43, 93-5, 93-41, and 93-31

FIG. 2 illustrates the comparison of the in vitro potency boost of Compounds 93-97, 93-43, 93-5, 93-41, and 93-31 at pH 6.9 vs 7.6 versus tissue infarct volume following ICV administration of these agents. The data represents the % of infarct volume determined for vehicle injected controls and potency boost measured as described above. The grey shadowed area indicates the area which defines the identified bounds of the criteria for improved drug performance. The drugs which fall within the bounds are those that have a mean (not error bars) within the grey blocked area.

The infarct volume was measured in C57B1/6 mice following a transient focal ischemic event as described above for each compound. Compounds 93-97, 93-43, 93-5, 93-41 and 93-31 were applied intracerebroventricularly (ICV; solid circles) as described above. Error bars are standard error of the mean (SEM). The potency boosts at pH 6.9 vs 7.6 for Compounds 93-5, 93-31, 93-41, 93-43, and 93-97 were calculated as described herein for oocytes expressing NR1/NR2B receptors.

Compounds 93-4, 93-5, 93-8, 93-31, 93-40, (−)MK801 and (+)MK801

FIG. 3 illustrates the comparison of the in vitro potency boost of Compounds 93-4, 93-5, 93-8, 93-31, 93-40 at pH 6.9 vs 7.6 versus tissue infarct volume. The data represents the actual infarct volume expressed as percent of that in vehicle injected control animals and potency boost was calculated as described above. The grey shadowed area indicates the area which defines the identified bounds of the criteria for improved drug performance. The drugs which fall within the bounds are those that have a mean (not error bars) within the grey blocked area.

The infarct volume was measured in C57B1/6 mice following a transient focal ischemic event as described above for each compound. Drug was applied by intraperitoneal injection (IP) as described above. Error bars are SEM. Infarct volume was inferred from the percent reduction in infarct volume for IP administration compared to paired controls. This was calculated as the product of the infarct volume expressed as percent of control infarct induced by drug in an independent experiment and the mean control infarct volume (mm3) for ICV experiments, which is shown as solid line (broken lines show mean control infarct+-SEM). The potency boosts at pH 6.9 vs 7.6 for Compounds 93-4, 93-5, 93-8, 93-31, and 93-40, (+) MK801 and (−) MK801 were calculated as described herein for oocytes expressing NR1/NR2B receptors.

Additional Compounds

FIG. 4 compares the in vitro potency boost at NR1/NR2A and NR1/NR2B of known compounds at pH 6.9 vs 7.6 versus percent control tissue infarct volume. The grey shadowed area indicates the area which defines the identified bounds of the criteria for improved drug performance. The drugs which fall within the bounds are those that have a mean (not error bars) within the grey blocked area.

Open symbols show the reduction in infarct volume by administration of CNS1102 (CN, aptiganel or Cerestat, Dawson et al., 2001), dextromethorphan (DM, Steinberg et al., 1995), dextrorphan (DX; Steinberg et al., 1995), levomethorphan (LM; Steinberg et al., 1995), (S) ketamine (KT; Proescholdt et al., 2001), memantine (MM; Culmsee et al. 2004), ifenprodil (IF, Dawson et al. 2001), CP101,606 (CP; Yang et al. 2003), AP7 (Swan and Meldrum, 1990), Selfotel (CGS19755, Dawson et al., 2001), (R)HA966 (HA; Dawson et al., 2001), remacemide (RE, Dawson et al., 2001), haloperidol (O'Neill et al., 1998), 7-Cl-kynurenic acid (CK, Wood et al., 1992) and stereoisomer of MK801 (+MK or −MK; Dravid et al., in preparation) as described in the literature in various rodent or rabbit ischemia models (see references below). Percent reduction in infarct was calculated from the ratio of the infarct volume in drug to that in control for all compounds except ketamine and 7-Cl-kynurenic acid, for which the percent reduction in neuronal density by drug was measured.

The potency boosts at pH 6.9 vs 7.6 for all compounds were calculated as described above for oocytes expressing either NR1/NR2A or NR1/NR2B receptors (see Table 3 and 4 for summary of numbers of experiments). The pH boost for ifenprodil (IF), CP101,606 (CP) were determined from them literature (Mott et al., 1998).

The number of mice examined for infarct volume is shown in Table 3. For potency boost measurements on NR1-1a/NR2B receptors, the number of frogs used and the largest number of oocytes tested at a single concentration at pH 6.9 and pH 7.6 are shown in Table 3. For determination of IC50 at each pH, multiple concentrations of each drug were tested. For potency boost measurements on NR1-1a/NR2A receptors, the number of frogs used and the largest number of oocytes tested at a single concentration at pH 6.9 and pH 7.6 are shown in Table 4.

TABLE 3
Number of repetitions of each experiment for data from
NR1/NR2B presented in FIGS. 1,2,3,4.

| | | NR2B potency boost | | |
|---|---|---|---|---|
| Infarcts | | assay in Xenopus oocytes | | |
| (# | | | Number of | Number of |
| mice) | | Number | oocytes at | oocytes at |
| icv | ip | of frogs | pH 6.9 | pH 7.6 |

NR2B selective antagonists

| | | | | | |
|---|---|---|---|---|---|
| NP93-4 | | 34 | 8 | 50 | 55 |
| NP93-5 | | 17 | 6 | 29 | 30 |
| NP93-8 | | 13 | 5 | 35 | 41 |
| NP93-31 | 35 | 20 | 6 | 45 | 48 |
| NP93-40 | | 18 | 5 | 47 | 41 |
| NP93-41 | 15 | | 5 | 44 | 47 |
| NP93-43 | 12 | | 5 | 52 | 44 |
| NP93-97 | 32 | | 5 | 40 | 30 |
| Haloperidol | | | 4 | 32 | 30 |
| channel blockers | | | | | |
| (+)MK801 | | 26 | 5 | 16 | 19 |
| (−)MK801 | | 31 | 5 | 18 | 24 |
| Cerestat | | | 5 | 30 | 30 |
| Dextromethorphan | | | 5 | 28 | 38 |
| Levomethorphan | | | 5 | 24 | 21 |
| Dextrorphan | | | 5 | 29 | 27 |
| Ketamine | | | 7 | 30 | 36 |
| Memantine | | | 5 | 22 | 24 |
| Remacemide | | | 2 | 17 | 12 |
| Glutamate-site blockers | | | | | |
| AP7 | | | 2 | 14 | 14 |
| Selfotel | | | 2 | 10 | 12 |
| (R)-CPP | | | 3 | 15 | 32 |
| Glycine-site blocker | | | | | |
| (R)HA966 | | | 2 | 13 | 12 |
| 7-Cl-kynurenic acid | | | 2 | 12 | 13 |

TABLE 4
Number of repetitions of each experiment for data from
NR1/NR2A presented in FIG. 4.

| | NR2A potency boost assay in Xenopus oocytes | | |
|---|---|---|---|
| | Number | Number of | Number of |
| | of frogs | oocytes at pH 6.9 | oocytes at pH 7.6 |
| channel blockers | | | |
| (+)MK801 | 6 | 39 | 42 |
| (−)MK801 | 5 | 22 | 32 |
| Dextromethorphan | 6 | 36 | 42 |
| Levomethorphan | 5 | 21 | 28 |
| Dextrorphan | 5 | 25 | 24 |
| Ketamine | 5 | 31 | 22 |
| Memantine | 5 | 28 | 23 |
| Remacemide | 2 | 21 | 18 |
| Glutamate-site blockers | | | |
| AP7 | 2 | 10 | 10 |
| Selfotel | 2 | 10 | 13 |
| (R)-CPP | 3 | 17 | 18 |
| Glycine-site blocker | | | |
| (R)HA966 | 2 | 14 | 12 |
| 7-Cl-kynurenic acid | 2 | 10 | 12 |

FIG. 1 represents a composite of FIGS. 2, 3 and 4. It illustrates that of the 24 compounds tested, 20 compound (83%) fall outside the area of the invention (denoted by the shaded area), indicating that over 80% of compounds tested fail to meet the identified standard for efefctive in vivo therapy. The grey shadowed area indicates the area that defines the identified bounds of the criteria for improved drug performance. The drugs which fall within the bounds are those that have a mean (not error bars) within the grey blocked area. The mean of Compounds 93-4, 93-5, 93-41, 93-31 fall within the shaded area for NR1/NR2B. The mean of (−) MK801 and ketamine fall within the shaded area for NR1/NR2A (FIG. 4).

In particular, in FIG. 1, the infarct volume was measured in C57B1/6 mice following a transient focal ischemic event as described above for compounds indicated by symbols. Drug was applied intracerebroventricularly (ICV; squares) or by intraperitoneal injection (IP; circles) as described above. Error bars are SEM. Infarct volume was directly measured as percent of the control infarct volume for IP administration compared to paired controls. Control is shown as solid line (broken lines show mean control infarct+/−SEM). Open symbols show the reduction in infarct volume by administration of CNS1102 (CN, aptiganel or Cerestat, Dawson et al., 2001), dextromethorphan (DM, Steinberg et al., 1995), dextrorphan (DX; Steinberg et al., 1995), levomethorphan (LM; Steinberg et al., 1995), (S) ketamine (KT; Proescholdt et al., 2001), memantine (MM; Culmsee et al. 2004), ifenprodil (IF, Dawson et al. 2001), CP101,606 (CP; Yang et al. 2003), AP7 (Swan and Meldrum, 1990), Selfotel (CGS19755, Dawson et al., 2001), (R)HA966 (HA; Dawson et al., 2001), remacemide (RE, Dawson et al., 2001), haloperidol (O'Neill et al., 1998), 7-Cl-kynurenic acid (CK, Wood et al., 1992) and stereoisomer of MK801 (+MK or −MK; Dravid et al., in preparation) as described in the literature in various rodent or rabbit ischemia models (see references below). Percent reduction in infarct was calculated from the ratio of the infarct volume in drug to that in control for all compounds except ketamine and 7-Cl-kynurenic acid, for which the percent reduction in neuronal density by drug was measured.

Also, in FIG. 1, the potency boosts at pH 6.9 vs 7.6 for compounds 93-4, 93-5, 93-8, 93-31, 93-40, 93-43, 93-97, (+) MK801, (−) MK801, and all other compounds was calculated as described above with numbers of observations reported in Tables 1 and 2. The pH boost for ifenprodil (IF), CP101,606 (CP) were determined from the literature (Mott et al., 1998).

### Example 5

### Evaluation in an In Vivo Model of Neuropathic Pain

Methods

Animals: Male Sprague-Dawley rats (Hsd:Sprague-Dawley®™SD®™, Harlan, Indianapolis, Ind., U.S.A.) weighing 100±10 g on surgery day and 250±10 g on testing day were housed three per cage. Animals had free access to food and water and were maintained on a 12:12 h light/dark schedule. The animal colony was maintained at 21° C. and 60% humidity. All experiments were conducted in accordance with the International Association for the Study of Pain guidelines and were approved by the University of Minnesota Animal Care and Use Committee.

Drugs and dosing solutions: The drugs were dissolved in 1% v/v DMSO and 66% v/v PEG 400 in distilled water. Compounds were administered by i.p. route.

Induction of chronic neuropathic pain: The Spinal Nerve Ligation (SNL) model (Kim and Chung 1992 Pain 50:355-63.) was used to induce chronic neuropathic pain. The animals were anesthetized with isoflurane, the left L5 transverse process was removed, and the L5 and L6 spinal nerves were tightly ligated with 6-0 silk suture. The wound was then closed with internal sutures and external staples. Wound clips were removed 10 days following surgery.

Mechanical allodynia testing: Baseline and post-treatment values for non-noxious mechanical sensitivity were evaluated using 8 Semmes-Weinstein filaments (Stoelting, Wood Dale, Ill., USA) with varying stiffness (0.4, 0.7, 1.2, 2.0, 3.6, 5.5, 8.5, and 15 g) according to the up-down method (Chaplan, Bach et al. 1994 J Neurosci Methods 53: 55-63). Animals were placed on a perforated metallic platform and allowed to acclimate to their surroundings for a minimum of 30 minutes before testing. The mean and standard error of the mean (SEM) were determined for each animal in each treatment group. Since this stimulus is normally not considered painful, significant injury-induced increases in responsiveness in this test are interpreted as a measure of mechanical allodynia.

Experimental design: von Frey baseline measurements were made 30 minutes and 24 hours prior to drug administration respectively. Additional von Frey measurements were made at 30, 60, 120 and 240 min. The timeline for testing is summarized below. The experimental groups were: ·vehicle (1% DMSO+66% PEG 400 in distilled water, i.p., 4 ml/kg, n=10)·30 mg/kg Compound 93-31 test (i.p., 4 ml/kg, n=10)·100 mg/kg Compound 93-31 test (i.p., 4 ml/kg, n=10)·30 mg/kg Compound 93-97 test (i.p., 4 ml/kg, n=10)·100 mg/kg Compound 93-97 test (i.p., 4 ml/kg, n=10)·100 mg/kg Gabapentin (i.p., 4 ml/kg, n=12) (Total rats: 62).

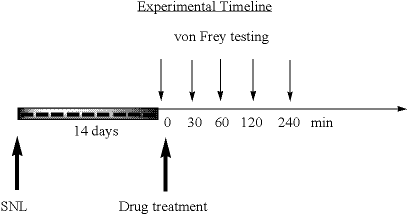

Blinding procedure: Drug solutions were administered by a separate experimenter who did not conduct the behavioral testing.

Data analysis: Statistical analyses were conducted using Prism™ 4.01 (GraphPad, San Diego, Calif., USA). Mechanical allodynia of the injured paw was determined by comparing values observed in the contralateral and ipsilateral paws within the vehicle group. Stability of vehicle group injured paw values over time was tested using the Friedman two-way analysis of variance by rank. Drug effect was analyzed at each time point by carrying out a Kruskal-Wallis one-way analysis of variance by rank followed by a Dunn's post hoc test.

Results

von Frey Testing

Testing for mechanical allodynia (von Frey) was initiated 14 days after SNL surgery. Tests were performed on both injured (ipsilateral) and normal (contralateral) paws at baseline (30 minutes before drug administration) and 30, 60, 120 and 240 minutes after a single drug administration.

At baseline all animals showed mechanical allodynia in the injured paw (Table 2). The level of impairment was comparable among groups, and throughout the study von Frey thresholds in the injured paw were significantly different from those observed in the normal paw of vehicle treated group (FIG. 6). FIG. 6 shows that animals in the vehicle group displayed significant mechanical allodynia for the entire duration of the study. Illustrated are mean±SEM (n=10) von Frey thresholds in the injured and normal paws of animals treated with vehicle. The difference between paws was significant at all time points (Mann-Whitney test). Compounds 93-31 and 93-97 had no effect on von Frey thresholds measured in the normal paw (FIGS. 7 and 8). FIG. 7 shows that Compound 93-31 did not alter von Frey thresholds in the normal paw. Illustrated are the mean±SEM (n=10-12) von Frey thresholds in the normal paw in animals treated with vehicle, gabapentin or 30 and 100 mg/kg doses of Compound 93-31 administered i.p. FIG. 8 shows that Compound 93-97 did not alter von Frey thresholds in the normal paw. Illustrated are the mean±SEM (n=10-12) von Frey thresholds in the normal paw in animals treated with vehicle, gabapentin or 30 and 100 mg/kg doses of 93-97 administered i.p.

TABLE 2
Injured paw - von Frey threshold values

| Treatment (mg/kg) | n | Baseline | 30 min | 60 min | 120 min | 240 min |
|---|---|---|---|---|---|---|
| Vehicle | 10 | 1.9 ± 0.3 | 2.3 ± 0.4 | 1.5 ± 0.3 | 1.4 ± 0.3 | 1.0 ± 0.2 |
| 93-31 (30) | 10 | 2.4 ± 0.4 | 1.9 ± 0.3 | 1.7 ± 0.1 | 0.8 ± 0.2 | 1.1 ± 0.3 |
| 93-31 (100) | 10 | 1.7 ± 0.3 | 9.3 ± 1.5 | 8.9 ± 1.7 | 1.9 ± 0.3 | 0.8 ± 0.2 |
| 93-97 (30) | 10 | 1.9 ± 0.2 | 1.6 ± 0.3 | 1.1 ± 0.1 | 0.9 ± 0.1 | 1.0 ± 0.2 |
| 93-97 (100) | 10 | 1.2 ± 0.1 | 1.1 ± 0.2 | 1.4 ± 0.3 | 1.0 ± 0.1 | 0.8 ± 0.2 |
| Gabapentin (100) | 12 | 1.9 ± 0.2 | 6.0 ± 1.1 | 11.1 ± 1.5 | 13.6 ± 0.9 | 6.6 ± 1.3 |

Values are mean ± SEM.

TABLE 3
Injured paw - Statistical analyses summary

| Treatment (mg/kg) | n | Baseline | 30 min | 60 min | 120 min | 240 min |
|---|---|---|---|---|---|---|
| Vehicle | 10 | — | — | — | — | — |
| 93-31 (30) | 10 | ns | ns | ns | ns | ns |
| 93-31 (100) | 10 | ns | p < 0.01 | p < 0.01 | ns | ns |
| 93-97 (30) | 10 | ns | ns | ns | ns | ns |
| 93-97 (100) | 10 | ns | ns | ns | ns | ns |
| Gabapentin (100) | 12 | ns | ns | p < 0.001 | p < 0.01 | p < 0.01 |
| Kruskal-Wallis | | p = 0.1182 | p < 0.0001 | p < 0.0001 | p < 0.0001 | p < 0.0001 |

ns = not significant vs. vehicle group.

Treatment with Compound 93-31 (100 mg/kg i.p.) generated observable analgesia at 30 and 60 min following its administration (FIG. 9). FIG. 9 illustrates that i.p. administration of Compound 93-31 (100 mg/kg) reduced mechanical allodynia. Shown are the mean±SEM (n=10-12) von Frey thresholds in the injured paw of animals treated with vehicle, gabapentin (reference compound) or 30 and 100 mg/kg doses of Compound 93-31 administered i.p. Post-hoc analysis (Dunn's test) showed significant pair-wise differences between Compound 93-31 (100 mg/kg) and vehicle groups at 30 and 60 minute (p<0.01). The effect of gabapentin at 60, 120 and 240 minutes was also significant (p<0.001, p<0.01, and p<0.01 respectively). There was no analgesic effect of 30 mg/kg of Compound 93-31, and 30 and 100 mg/kg of Compound 93-97 at any time point studied. Statistical analysis of the vehicle group in this study indicated that there was no significant difference in von Frey threshold between baseline and at 30, 60 120 and 240 minute time point (Friedman two-way ANOVA).

In addition, Compound 93-97 administered i.p. failed to attenuate mechanical allodynia in SNL rat. FIG. 10 shows that i.p. administration of Compound 93-97 (30 and 100 mg/kg) showed no effect on von Frey thresholds. Illustrated are the mean±SEM (n=10-12) von Frey thresholds in the injured paw of animals treated with vehicle, gabapentin (reference compound), or 30 and 100 mg/kg of Compound 93-97 administered i.p. The effect of gabapentin at 60, 120 and 240 minutes was also significant (p<0.001, p<0.01, and p<0.01 respectively).

Some side effects were observed in the group of animals tested in this study (8 out of 62 animals). The side effects observed were writhing and stretching (8 observations). These side effects were most commonly seen for the first few minutes (˜5 minutes) following i.p. drug administration. Stretching/writhing was seen in all study groups including those animals treated with vehicle i.p. (3/10) and did not appear to be dependent on drug dose. The severity of these side effects was modest and did not interfere with the endpoint measurement enough to exclude the animals from the study. Table 1 summarizes the side effects observed in this study. Some side effects were observed while measuring the endpoints. The most common was stretching/writhing which may be a sign of some visceral pain or hypersensitivity. This was seen in the vehicle and drug treated i.p. groups. It seems likely to be associated with i.p. administration of the vehicle in a subset of animals. This seemed relatively rare, short lived (<5 min), and the magnitude was not large enough to interfere with measurement of the endpoint.

TABLE 1
Side Effects

| Vehicle | 3/10 stretching/writhing (for first 5 min) |
|---|---|
| 93-31 (30 mg/kg) | 0/10 stretching/writhing |
| 93-31 (100 g/kg) | 1/10 stretching/writhing (for first 5 min) |
| 93-97 (30 mg/kg) | 2/10 stretching/writhing (for first 5 min) |
| 93-97 (100 g/kg) | 1/10 stretching/writhing (for first 5 min) |
| Gabapentin (100 mg/kg) | 1/10 stretching/writhing (for first 5 min) |

Compound 93-31 appeared to attenuate mechanical allodynia in the SNL model of neuropathic pain when administered i.p. at 100 mg/kg. Compound 93-97 failed to attenuate mechanical allodynia in SNL rats at the doses tested (30 and 100 mg/kg) in this study. Compound 93-31 (100 mg/kg) appeared to have a faster onset (30 min) and shorter duration of action (60 min) than did the reference compound gabapentin (100 mg/kg). The peak threshold observed in animals treated with the 100 mg/kg dose of Compound 93-31 was approximately half of that seen in the normal paw. Assuming complete reversal may be achieved with higher doses of Compound 93-31, this suggests the ED50 is approximately 100 mg/kg.

### Example 6

### pH Dependence of Selected Compound

A series of n-alkyl derivatives was tested for pH dependence.

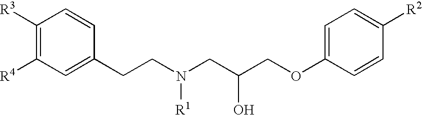

| R1 | IC50 pH 7.6/IC50 pH 6.9 |
|---|---|
| —H | 3 |
| —CH3 | 6 |
| —CH2CH3 | 8 |
| —CH2CH2CH3 | 6 |
| —CH2CH2CH2CH3 | 17 |
| —CH2CH2CH2CH2CH3 | 3 |

While the foregoing invention has been described in some detail for purposes of clarity and understanding, it will be appreciated by one skilled in the art from a reading of this disclosure that various changed in form and detail can be made without departing from the true scope of the invention.

We claim:

1. A process to identify a compound that is useful to treat or prevent ischemic or hypoxic injury in a mammal comprising (i) assessing the potency boost of the compound at physiological pH versus disorder-induced low pH in a cell by repeating the potency boost experiment at least 5 times such that the 95% confidence interval does not change more than 15% with the addition of a new experiment; (ii) testing the compound in an animal model of transient focal ischemia and measuring the effect of the compound on the infarct volume by repeating the experiment at least 12 times such that the 95% confidence interval does not change more than 5% with the addition of a new experiment; (iii) selecting a compound that has a potency boost of at least 5 according to step (i) and at least a 30% decrease in infarct volume according to step (ii).

2. A process to select a compound to treat or prevent a disorder that lowers the pH wherein the compound (i) exhibits a potency boost of at least 5 as determined in experiments in which the potency boost of the compound is assessed at physiological pH versus disorder-induced low pH in a cell by repeating the potency boost experiments at least 5 times such that the 95% confidence interval does not change more than 15% with the addition of a new experiment and (ii) exhibits at least a 30% decrease in infarct volume as measured in an animal model of focal ischemia as determined by repeating the experiment at least 12 times such that the 95% confidence interval does not change more than 5% with the addition of a new experiment.

3. The process of claim 1 wherein the mammal is a human.

4. The process of claim 1 or 2, wherein the cell expresses a glutamate receptor.

5. The process of claim 4, wherein the glutamate receptor is an NMDA receptor.

6. The process of claim 5, wherein the NMDA receptor comprises an NR1 subunit and at least one NR2 subunit selected from the group consisting of NR2A, NR2B, NR2C, and NR2D or any combination thereof.

7. The process of claim 5, wherein the NMDA receptor comprises an NR1 subunit and an an NR2 or NR3 subunit selected from the group consisting of NR2A, NR2B, NR2C, NR2D, NR3A, and NR3B or any combination thereof.

8. The process of claim 4, wherein the glutamate receptor comprises glutamate receptor subunits selected from the group consisting of GluR1, GluR2, GluR3, GluR4, GluR5, GluR6, GluR7, KA1, KA2, delta-1 and delta-2.

9. The compound of claim 1 or 2 wherein the compound is: 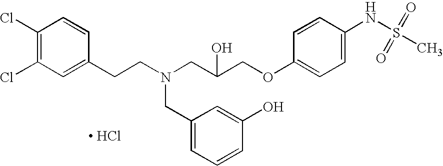
as well as pharmaceutically acceptable salts, esters, enantiomers, enantiomeric mixtures, and mixtures thereof.

10. The compound of claim 9 wherein the compound is 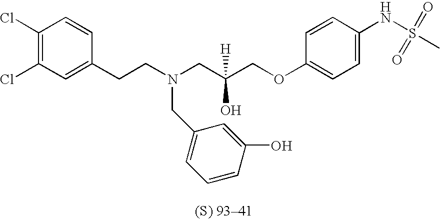
as well as pharmaceutically acceptable salts thereof.

11. The process of claim 2, wherein the disorder is ischemic or hypoxic injury

12. The process of claim 2, wherein the disorder is neuropathic pain or related disorder.

13. The process of claim 2, wherein the disorder is a brain tumor.

14. The process of claim 2, wherein the disorder is epilepsy.

15. The process of claim 2, wherein the disorder is a neurodegenerative disease.

16. The process of claim 11, wherein the ischemic or hypoxic injury is selected from the group consisting of: stroke, vasospasm after subarachnoid hemorrhage, traumatic brain injury, cognitive deficit after bypass surgery, cognitive deficit after carotid angioplasty; and ischemia following hypothermic circulatory arrest.

17. The process of claim 12, wherein the neuropathic pain or related disorder is selected from the group consisting of: peripheral diabetic neuropathy, postherpetic neuralgia, complex regional pain syndromes, peripheral neuropathies, cancer neuropathic pain, chemotherapy-induced neuropathic pain, neuropathic low back pain, HIV neuropathic pain, trigeminal neuralgia, and central post-stroke pain.

18. The process of claim 15, wherein the neurodegenerative disease is selected from the group consisting of: Parkinson's disease, Alzheimer's disease, Huntington's disease and Amyotrophic Lateral Sclerosis.

19. The process of claim 16, wherein the ischemic or hypoxic injury is stroke.

20. The process of claim 16, wherein the ischemic or hypoxic injury is vasospasm after subarachnoid hemorrhage.

21. The process of claim 1 or 2, wherein the compound does not cause cognitive impairment.

22. The process of claim 21, wherein the cognitive impairment is psychotic-like symptoms.
